(19) United States
(12) Patent Application Publication
(10) Pub. No.: US 2019/0284259 A1
(43) Pub. Date: Sep. 19, 2019

(54) Monoclonal Antibodies to B Virus And Their Use For Identification Of B Virus Specific Reactive Peptides

(71) Applicant: Georgia State University Research Foundation, Atlanta, GA (US)

(72) Inventors: David Katz, Atlanta, GA (US); Julia Hilliard, Atlanta, GA (US)

(21) Appl. No.: 16/319,718

(22) PCT Filed: Jul. 20, 2017
(86) PCT No.: PCT/US17/43005

§ 371 Date: Jan. 22, 2019

Related U.S. Application Data
(60) Provisional application No. 62/365,424, filed on Jul. 22, 2016.

(51) Int. Cl. C07K 16/08; A61K 39/12

(52) U.S. Cl. CPC C07K 16/087; A61K 39/12; C12N 2710/16034; C07K 2317/34; C07K 2317/33

(57) ABSTRACT

Embodiments of the disclosure relate generally to B virus epitopes, and more specifically to B virus epitopes and monoclonal antibodies (MABs) that can distinguish between different B viruses specific to different hosts and can further neutralize said viruses; compositions containing the epitopes and MABs and nucleic acids encoding the epitopes and MABs; and specific B virus peptides and MABs that can be used in diagnostic assays or as sources for vaccines or other therapeutic interventions.

### CROSS-REFERENCE TO RELATED APPLICATIONS

This application claims priority to U.S. Provisional Application No. 62/365,424, filed on 22 Jul. 2016, the disclosure of which is herein incorporated by reference in its entirety.

### GOVERNMENT SPONSORSHIP

This invention was made with government support under Grant Nos. P50 RR05062 and R01 RR03163 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on 18 Jul. 2017, is named 026552.000025_SL.txt and is 29,138 bytes in size.

### BACKGROUND OF THE DISCLOSURE

### 1. Field of the Invention

Embodiments of the disclosure relate generally to B virus epitopes, and more specifically to B virus epitopes and monoclonal antibodies (MABs) that can distinguish between different B viruses specific to different hosts and can further neutralize said viruses; compositions containing the epitopes and MABs and nucleic acids encoding the epitopes and MABs; and specific B virus peptides and MABs that can be used in diagnostic assays or as sources for vaccines or other therapeutic interventions.

### 2. Background and Summary

B virus (Macacine herpesvirus 1) is part of the genus Simplexvirus in the Alphaherpesviridae subfamily and family Herpesviridae that consists of a number of related neurotropic herpes viruses. A list of the simplexviruses and their natural hosts is shown in Table 1. These viruses cross-react immunologically to a different extent depending on the assay used (1, 2, 3, 4).

TABLE 1
Primate hosts and their natural herpesviruses:

| Primate Natural Host | Simplexvirus |
|---|---|
| Macaques (Macaca spp) | B virus (Macacine herpesvirus 1) |
| Baboons (Papio spp) | HVP2 (Papiine herpesvirus 2) |
| African green monkeys | SA8 (Cercopithecine herpesvirus 2) |
| (Cercopithecus aethiops) | |
| Langurs (Presbytis spp) | HVL-Herpesvirus-Langur (*) |
| Mangabeys (Cercocebus atys) | HVM-Herpesvirus-Mangabey (*) |
| Humans (Homo sapiens) | HSV 1 and 2 (Human herpesvirus 1 and 2) |

* Provisional name-not officially classified

Different B virus isolates from different macaque species are considered to be very close and almost indistinguishable immunologically. However, in a study in which B virus isolates from four macaque species viz., rhesus, cynomolgus, pigtail and Japanese were compared, it was established that the isolates could be grouped into three B virus genotypes or strains. Isolates from rhesus and Japanese macaques were both classified as belonging to one genotype and isolates from pigtail and cynomolgus macaques were grouped each into two distinct genotypes.

Primary B virus infections in the natural host (macaques), establish a latent infection in the sensory ganglia that innervate the region of the original inoculation site. Subsequent reactivation, caused by stress is accompanied by unpredictable events of virus shedding. If there are symptoms, they are often mild and transient, similar to symptoms in humans infected with HSV-1 and HSV-2. Cross-species infection with B virus is known to be associated usually with enhanced virulence resulting in serious clinical disease (3,4,5,6,7). Clinical symptoms in humans infected with B virus generally include massive systemic disease and encephalitis. The fatality rate in untreated humans may reach 80%. There is evidence that some humans may recover from infection and harbor the virus latently, particularly following administration of anti viral drugs early after infection. Also, recent evidence indicates that a human latently infected with B virus has experienced a symptomatic reactivation of the virus on two occasions (8).

Macaques can also transmit B virus to other non-human primate species, e.g., owl monkey, marmosets, and gibbon apes, all of which share similarly high mortality rates to humans (6,9,10).

Early and accurate diagnosis of B virus infections in macaques, other non-human primates and humans is extremely important for the maintenance of captive monkey colonies and as a tool for clinicians that enables early antiviral intervention and the saving of human lives. Direct virus culture or detection in clinical specimens using an immunological method or PCR would be the method of choice, however, since virus shedding is rare, diagnosis of B virus infection in monkeys and humans must be based mainly on antibody detection (serology). This limitation postpones diagnosis to at least one or two weeks after infection when antibodies are detectable. It is therefore of utmost importance that B virus diagnostic serology is as rapid and accurate as possible. Serological diagnosis of B virus infection in macaques is relatively simple since no other cross-reacting virus is known to infect them. Diagnosing a B virus infection in humans is more complicated than in macaques since quite often humans posses cross-reacting antibodies to HSV-1 and/or HSV-2.

Currently, serological diagnosis of B virus infection in macaques is commonly based on two principal tests: the titration ELISA (tELISA) that serves as a screening test and the western blot analysis (WBA) that is used for confirming tELISA-positive results (11). The tELISA cannot be used for serological diagnosis of a zoonotic B virus infection in humans because it does not discriminate between B virus, HSV-1 or HSV-2 infections. Alternatively, WBA and competition ELISA (cELISA) are used for B virus differential serological diagnosis in humans. WBA enables detection of antibodies to denatured virus polypeptides that are present in the infected cell lysates. The test results in a pattern of bands that is often unique for a B virus infection and can often differentiate B virus induced antibodies from HSV induced antibodies. It is because of this analytical power, that WBA was considered as a “gold standard” and a confirmatory test for the screening tELISA. The strategy of using WBA as the gold standard resulted in a substantial amount of “tELISA-false positives” because many of the low titer tELISA results turn out to be negative or indeterminate by WBA. However, it was observed that some of the ELISA “false-positive” monkeys sero-converted later and were confirmed to be positive by WBA. This indicated that the tELISA detected antibody responses earlier than WBA and that using WBA as the gold standard for tELISA might have delayed some early, true positive, diagnoses of infection. In addition, it is known that the antigen denaturation process that is part of the WBA procedure prevents detection of antibodies to non-linear, conformational, epitopes. This may explain the higher sensitivity of the ELISA in which denaturation of antigens is less prominent (12,13,14,15). The cELISA is capable of identifying specific antibodies to each one of the 3 simplexviruse that may infect humans (HSV-1, HSV-2 and B virus) in a mixture of cross-reacting antibodies. (16). However, cELISA is relatively complicated to perform, especially for routine purposes. Results are sometimes obscured because of possible differences in the relative amount of antibodies to each of the viruses in a mixed infection. Also, non-specific competition results cannot be ruled out since the competing antigens and the antigens adsorbed to the wells are from the same source of similar infected cell lysates. Despite of the drawbacks of the cELISA for routine diagnostics, the test proves that specific, unidentified, B virus epitopes do exist and therefore chemical identification of these should be possible. Antibodies detected by ELISA and WBA react with a mixture of B virus specific antigens that are adsorbed onto polystyrene wells of microplates or nitrocellulose membranes. In these infected cell lysates the majority of virus-induced infected cell polypeptides (ICPs) and virus structural antigens are represented but it is expected that some specific epitopes are precluded from reacting with antibodies as a result of being blocked by nearby cross-reacting epitopes bound to non-specific antibodies (13,17). The current indirect approaches for diagnosing B virus infections that utilize complex mixtures of infected cell antigens require considerable time, expense and a BSL-4 biocontainment facility limiting the number of facilities that can perform these evaluations. As a result many macaque colonies managers have had to use heterologous viruses for identification of B virus antibodies, an approach that compromises these potential invaluable resources (12). Using undefined mixture of antigens results at times in complex data that can be difficult to interpret and therefore hamper the necessary unequivocal evaluation of B virus infections in humans and non-human primates. The inventors therefore searched for other strategies for identification and production of B virus specific antigens that can provide a source of reagents for which genetic material is available to provide an endless, consistent supply of the antigen, undiluted by proteins, peptides, or amino acid sequences that reduce the sensitivity and specificity of the assays in which these are used. One such strategy was the production of B virus recombinant antigens (17). B virus recombinant proteins were recently evaluated as reagents for specific differential diagnosis. Using major recombinant glycoproteins that were produced in the inventors' lab (gB, gC, gD and gGm) only glycoprotein G (gGm) reacted specifically with sera from B virus infected macaques. Unfortunately, antibodies to this protein are often not induced. The other three, and gB in particular cross reacted with antibodies to other simplexviruses (13,17). In a recent study in which the tELISA, WBA and a recombinant based ELISA were compared, it was concluded that the recombinant-ELISA performed as well or better then WBA and that it can serve as a confirmatory assay instead of WBA for detecting antibodies in macaque sera (13).

However, the selected recombinant antigens did not enable the construction of a B virus specific ELISA for testing human sera. The inventors therefore decided to employ a different strategy for identification of B virus specific antigens. One of the best tools for specific antigen identification and mapping are monoclonal antibodies (18) in combination with the innovative use of phage display technologies. The combined techniques enable genetic reproduction of an unlimited source of diagnostic reagents (19,20). These methods may lead to the discovery of linear, discontinuous or conformational epitopes or mimitopes that are specifically recognized by the monoclonal antibodies (MABs). Another technique that is limited for detection of linear epitopes, is screening against overlapping peptide arrays that are synthesized based on published amino acid sequence of a polypeptide of interest (30, 31). Although MABs to B virus antigens were produced by other investigators, (21,22) no one has examined the potential of these antibodies for differential B virus diagnosis in humans or for the identification of specific B virus peptides that can be used in diagnostic assays or as sources for vaccines or other therapeutic interventions.

Herein is described the production and characterization of anti B virus MABs and their use for identification of reactive peptides by the phage display technique and by overlapping peptide arrays. The present disclosure also describes novel antibodies that bind to B viruses and can be used in therapeutic and/or diagnostic methods.

### BRIEF SUMMARY OF THE INVENTION

As specified in the Background Section, there is a great need in the art to identify reagents specific for certain B virus subtypes and use this understanding to develop novel compositions and assays for differential diagnosis of various B viruses in monkeys and humans, treatment of said B viruses, and identification of peptides enabling said differential diagnosis. The disclosure satisfies this and other needs. Embodiments of the disclosure relate generally to B virus epitopes and monoclonal antibodies (MABs) that can distinguish between different B viruses specific to different hosts and can further neutralize said viruses; compositions containing the epitopes and MABs and nucleic acids encoding the epitopes and MABs; and specific B virus epitopes and MABs that can be used in diagnostic assays or as sources for vaccines or other therapeutic interventions. The disclosure further provides methods, compositions, kits and articles of manufacture related to such B virus epitopes and MABs.

In one aspect, the disclosure includes any of the monoclonal antibodies (MABs) as described in Table 1. In an embodiment of this aspect, the disclosure further comprises a nucleic acid encoding any of the MABs described in Table 1. In another embodiment, the disclosure includes a vector comprising said nucleic acid. In yet another embodiment, the disclosure includes a host cell comprising said vector. In a further embodiment, the host cell may be a bacterial, yeast or eukaryotic cell.

In another aspect, the disclosure includes any of the B virus epitopes as described in Table 7 (e.g., SEQ ID NOs: 13-85) and homologous variants thereof (e.g., amino acid sequences having at least 95%, 96%, 97%, 98%, 99%, or 100% sequence identity to any of SEQ ID NOs: 13-85). In an embodiment of this aspect, the disclosure further comprises a nucleic acid encoding any of the B virus epitopes and homologous variants thereof. In another embodiment, the disclosure includes a vector comprising said nucleic acid. In yet another embodiment, the disclosure includes a host cell comprising said vector. In a further embodiment, the host cell may be a bacterial, yeast or eukaryotic cell. In another embodiment, the disclosure includes a monoclonal antibody recognizing said B virus epitope (e.g., SEQ ID NOs: 13-85) and homologous variants thereof (e.g., amino acid sequences having at least 95%, 96%, 97%, 98%, 99%, or 100% sequence identity to any of SEQ ID NOs: 13-85). In yet another embodiment, the disclosure includes a recombinant and/or chimeric protein comprising at least one B virus epitope (e.g., SEQ ID NOs: 13-85) and homologous variants thereof (e.g., amino acid sequences having at least 95%, 96%, 97%, 98%, 99%, or 100% sequence identity to any of SEQ ID NOs: 13-85). In a further embodiment, the disclosure includes a nucleic acid encoding said recombinant and/or chimeric protein. In a further embodiment, the disclosure includes a host cell comprising said vector. In a further embodiment, the host cell may be a bacterial, yeast or eukaryotic cell.

In another aspect, the disclosure includes a pharmaceutical composition comprising any composition of the above embodiments and combinations thereof. In a further embodiment, the pharmaceutical composition further comprises a carrier and/or excipients and/or additional therapeutic agent. In any of the above embodiments, the pharmaceutical composition comprises a therapeutically effective amount of any of the compositions of the above embodiments. In any of the above embodiments, the pharmaceutical composition is a vaccine.

In another aspect, the disclosure includes the use of any composition of the above embodiments to treat a simplexvirus infection in a subject in need thereof.

In yet another aspect, the disclosure includes a method of treatment of a simplex virus infection in a subject in need thereof, comprising administering any composition of the above embodiments. In one embodiment, the method comprises diagnostic assay to determine whether the subject has a simplexvirus infection using the antibodies of the disclosure. In a further embodiment, the diagnostic assay can provide a differential diagnosis of the simplexvirus infection from the group comprising Rh-BV, Cyno-BV, PT-BV, Jap-BV, HSV-1 and/or HSV-2. In a further embodiment of any of the above methods, the method can comprise administering a vaccine comprising any of the pharmaceutical compositions of the disclosure.

In another embodiment of any of the above methods, the method comprises treatment of Rh-BV, Jap-BV, PT-BV, and/or Cyno-BV infections with any compositions according to the above embodiments, preferably comprising one or more of 7H1.G5, 18D10.F2.A4, 2G12.D12.D4, 6E10.D7, 5D10.C9, 5E10.C10, 7G9.E3, 10F9.F1, 7F7.G7, 5A2, 4E11, 9F1, 3H6, 7G2, 7G8, and/or 1G3; B virus epitopes recognized by said MABs; and/or other MABs specific to the B virus epitopes recognized by said MABs. In another embodiment of any of the above methods, the method comprises neutralizing the B virus by administration of a composition according to any of the above embodiments comprising: 5A2 and/or 4E11 MABs; B virus epitopes recognized by said MABs; and/or other MABs specific to the B virus epitopes recognized by said MABs.

In another aspect, the disclosure includes a method of diagnosing a subject with a simplexvirus infection comprising use of the MABs described in Table 1. In an embodiment, the simplexvirus infection is a B virus infection, a HSV-1 infection, and/or a HSV-2 infection.

In another aspect, the disclosure includes a method of diagnosing a subject with a simplexvirus infection comprising use of MABs recognizing the B virus epitopes described in Table 7 (e.g., SEQ ID NOs: 13-85) and homologous variants thereof (e.g., amino acid sequences having at least 95%, 96%, 97%, 98%, 99%, or 100% sequence identity to any of SEQ ID NOs: 13-85). In an embodiment, the simplexvirus infection is a B virus infection, a HSV-1 infection, and/or a HSV-2 infection.

In an embodiment of any of the diagnostic methods, the method further comprises the steps of:

(i) taking a bodily fluid sample (e.g., blood, serum, plasma, urine, saliva, and/or CSF) from a subject;

(ii) determining whether the sample contains a simplexvirus; and

(iii) determining whether the subject is infected with a simplexvirus.

In a further embodiment, the method is used to make a differential diagnosis among Rh-BV, Cyno-BV, PT-BV, Jap-BV, HSV-1, and/or HSV-2.

In another embodiment of any of the above diagnostic methods, the method allows differential diagnosis of Rh-BV and Jap-BV strains by using MABs 12F5.C1 and/or 12G9.G5 as described in Table 1, and/or other MABs recognizing the B virus epitopes of these MABs as described in Table 7 (e.g., SEQ ID NOs: 13-85) and homologous variants thereof (e.g., amino acid sequences having at least 95%, 96%, 97%, 98%, 99%, or 100% sequence identity to any of SEQ ID NOs: 13-85).

In another embodiment of any of the above diagnostic methods, the method allows specific diagnosis of one or more of Rh-BV, Cyno-BV, PT-BV, and/or Jap-BV, and not HSV-1 and/or HSV-2, by using one or more of MABs 1G3, 1G3.C1, 9F1, 3H6, and/or 7G2, and/or other MABs recognizing the B virus epitopes of these MABs as described in Table 7 (e.g., SEQ ID NOs: 13-85) and homologous variants thereof (e.g., amino acid sequences having at least 95%, 96%, 97%, 98%, 99%, or 100% sequence identity to any of SEQ ID NOs: 13-85).

In another embodiment of any of the above diagnostic methods, the method allows specific diagnosis of one or more of Rh-BV, Cyno-BV, PT-BV, and/or Jap-BV, and not HSV-1 and/or HSV-2, by using one or more of MABs 2G12.D12.D4 and/or 6E10.D7, and/or other MABs recognizing the B virus epitopes of these MABs as described in Table 7 (e.g., SEQ ID NOs: 13-85) and homologous variants thereof (e.g., amino acid sequences having at least 95%, 96%, 97%, 98%, 99%, or 100% sequence identity to any of SEQ ID NOs: 13-85).

In another embodiment of any of the above diagnostic methods, the method allows specific diagnosis of one or more of Rh-BV, Cyno-BV, PT-BV, and/or Jap-BV by using one or more of MABs 5E10.C10, 7G8, and/or 7F7.G7, and/or other MABs recognizing the B virus epitopes of these MABs as described in Table 7 (e.g., SEQ ID NOs: 13-85) and homologous variants thereof (e.g., amino acid sequences having at least 95%, 96%, 97%, 98%, 99%, or 100% sequence identity to any of SEQ ID NOs: 13-85).

In another embodiment of any of the above diagnostic methods, the method allows differential diagnosis of one or more of Rh-BV, Cyno-BV, PT-BV, Jap-BV, HSV-1 and/or HSV-2 relative to other simian simplexviruses and human simplexviruses by using one or more of MABs 18.D10.F2.A4, 7H1.G5, 5D1O.C9, 7G9.E3, 5A2, and/or 4E11, and/or other MABs recognizing the B virus epitopes of these MABs as described in Table 7 (e.g., SEQ ID NOs: 13-85) and homologous variants thereof (e.g., amino acid sequences having at least 95%, 96%, 97%, 98%, 99%, or 100% sequence identity to any of SEQ ID NOs: 13-85).

In a further embodiment of any of the above diagnostic methods, the step of determining whether the sample contains simplexviruses involves a hybridization assay.

In a further embodiment of any of the above diagnostic methods, the method further comprises selecting a therapeutic composition and/or method based on the results of step (iii).

The present disclosure provides binding molecules, in particular antibodies, which bind specifically and/or differentially to B viruses; representative anti-B virus antibodies of the disclosure may comprise at least one of the antibody variable region amino acid sequences shown in SEQ ID NOs: 1-6 (and/or encoded by the nucleic acid sequences shown in SEQ ID NOs: 7-12, or individual CDRs thereof or related CDR sequences, as specified in more detail below.

Specifically the present disclosure provides antibodies that bind specifically and/or differentially to B viruses, more specifically monoclonal antibodies. The present disclosure also provides B virus-binding fragments that are at least 95%, 96%, 97%, 98%, 99%, or 100% identical to any of SEQ ID NOs: 1-6 or 7-12.

Included in the present disclosure are anti-B virus antibodies and fragments thereof that bind specifically and/or differentially to B viruses and comprise a light chain variable region having an amino acid sequence that is at least 95%, 96%, 97%, 98%, 99%, or 100% identical to any of SEQ ID NOs: 1, 3 and 5, or is a B virus-binding fragment or a homologous variant thereof, of an antibody comprised in said sequences. Alternatively, the anti-B virus antibodies and fragments thereof may be encoded by a nucleic acid having a nucleic acid sequence that is at least 95%, 96%, 97%, 98%, 99%, or 100% identical to any of SEQ ID NOs: 7, 9 and 11.

Also included are anti-B virus antibodies and fragments thereof that bind specifically and/or differentially to B viruses and comprise a heavy chain variable region having an amino acid sequence that is at least 95%, 96%, 97%, 98%, 99%, or 100% identical to any of SEQ ID NOs: 2, 4, and 6, or is a B virus-binding fragment or a homologous variant thereof, of an antibody comprised in said sequences. Alternatively, the anti-B virus antibodies and fragments thereof may be encoded by a nucleic acid having a nucleic acid sequence that is at least 95%, 96%, 97%, 98%, 99%, or 100% identical to any of SEQ ID NOs: 8, 10 and 12.

The anti-B virus antibodies of the disclosure include an anti-B virus antibody or fragment thereof or a B virus-binding fragment or homologous variant thereof as described above, which is selected from the group consisting of a chimeric antibody, a CDR-grafted or humanized antibody, a single chain antibody, a fusion protein, and a human antibody.

In certain embodiments, an antibody or an antibody fragment that binds to a B virus or a fragment thereof is provided, wherein the antibody or fragment thereof comprises a variable domain comprising a heavy chain (VH) comprising the amino acid sequence of SEQ ID NO: 2, 4, or 6 and a light chain (VL) comprising the amino acid sequence of SEQ ID NO: 1, 3, or 5. Alternatively, the VH region may be encoded by a nucleic acid comprising the nucleic acid sequence of SEQ ID NO: 8, 10, or 12 and a light chain (VL) comprising the amino acid sequence of SEQ ID NO: 7, 9, or 11.

In certain embodiments, an antibody or an antibody fragment that binds to a B virus or a fragment thereof is provided, wherein the antibody or fragment thereof comprises (a) a VH sequence having at least 95%, 96%, 97%, 98%, 99%, or 100% sequence identity to the amino acid sequence of SEQ ID NO: 2, 4, or 6; or (b) a VL sequence having at least 9595%, 96%, 97%, 98%, 99%, or 100% sequence identity to the amino acid sequence of SEQ ID NO: 1, 3, or 5.

In other embodiments, an an antibody or an antibody fragment that binds to a B virus or a fragment thereof is provided, wherein the antibody or fragment thereof comprises (a) a VH sequence encoded by a nucleic acid having at least 95%, 96%, 97%, 98%, 99%, or 100% sequence identity to the nucleic acid sequence of SEQ ID NO: 8, 10, or 12; or (b) a VL sequence having at least 95%, 96%, 97%, 98%, 99%, or 100% sequence identity to the amino acid sequence of SEQ ID NO: 7, 9, or 11.

In one embodiment, an antibody or an antibody fragment that binds to a B virus or a fragment thereof is provided, wherein the antibody comprises a VH comprising the amino acid sequence of SEQ ID NO: 2 (or the VH region may be encoded by a nucleic acid comprising the nucleic acid sequence of SEQ ID NO: 8). In certain embodiments, the antibody or fragment thereof further comprises a VL comprising the amino acid sequence of SEQ ID NO: 1 (or the VL region may be encoded by a nucleic acid comprising the nucleic acid sequence of SEQ ID NO: 7). In certain embodiments, the antibody or fragment thereof comprises a VH comprising the amino acid sequence of SEQ ID NO: 2 and a VL comprising the amino acid sequence of SEQ ID NO: 1. In certain embodiments, the antibody or fragment thereof comprises a VH encoded by a nucleic acid comprising the nucleic acid sequence of SEQ ID NO: 8 and a VL region encoded by a nucleic acid comprising the nucleic acid sequence of SEQ ID NO: 7.

In one embodiment, an antibody or an antibody fragment that binds to a B virus or a fragment thereof is provided, wherein the antibody comprises a VH comprising the amino acid sequence of SEQ ID NO: 4 (or the VH region may be encoded by a nucleic acid comprising the nucleic acid sequence of SEQ ID NO: 10). In certain embodiments, the antibody or fragment thereof further comprises a VL comprising the amino acid sequence of SEQ ID NO: 3 (or the VL region may be encoded by a nucleic acid comprising the nucleic acid sequence of SEQ ID NO: 9). In certain embodiments, the antibody or fragment thereof comprises a VH comprising the amino acid sequence of SEQ ID NO: 4 and a VL comprising the amino acid sequence of SEQ ID NO: 3. In certain embodiments, the antibody or fragment thereof comprises a VH encoded by a nucleic acid comprising the nucleic acid sequence of SEQ ID NO: 10 and a VL region encoded by a nucleic acid comprising the nucleic acid sequence of SEQ ID NO: 9.

In one embodiment, an antibody or an antibody fragment that binds to a B virus or a fragment thereof is provided, wherein the antibody comprises a VH comprising the amino acid sequence of SEQ ID NO: 6 (or the VH region may be encoded by a nucleic acid comprising the nucleic acid sequence of SEQ ID NO: 12). In certain embodiments, the antibody or fragment thereof further comprises a VL comprising the amino acid sequence of SEQ ID NO: 5 (or the VL region may be encoded by a nucleic acid comprising the nucleic acid sequence of SEQ ID NO: 11). In certain embodiments, the antibody or fragment thereof comprises a VH comprising the amino acid sequence of SEQ ID NO: 6 and a VL comprising the amino acid sequence of SEQ ID NO: 5. In certain embodiments, the antibody or fragment thereof comprises a VH encoded by a nucleic acid comprising the nucleic acid sequence of SEQ ID NO: 12 and a VL region encoded by a nucleic acid comprising the nucleic acid sequence of SEQ ID NO: 11.

In one embodiment, an antibody or an antibody fragment that binds to a B virus or a fragment thereof is provided, wherein the antibody comprises a VH comprising an amino acid sequence having at least 95%, 96%, 97%, 98%, 99%, or 100% sequence identity to the amino acid sequence of SEQ ID NO: 2 (or the VH region may be encoded by a nucleic acid comprising a nucleic acid sequence having at least 95%, 96%, 97%, 98%, 99%, or 100% sequence identity to the nucleic acid sequence of SEQ ID NO: 8). In certain embodiments, the antibody or fragment thereof further comprises a VL comprising an amino acid sequence having at least 95%, 96%, 97%, 98%, 99%, or 100% sequence identity to the amino acid sequence of SEQ ID NO: 1 (or the VL region may be encoded by a nucleic acid comprising a nucleic acid sequence having at least 95%, 96%, 97%, 98%, 99%, or 100% sequence identity to the nucleic acid sequence of SEQ ID NO: 7). In certain embodiments, the antibody or fragment thereof comprises a VH comprising an amino acid sequence having at least 95%, 96%, 97%, 98%, 99%, or 100% sequence identity to the amino acid sequence of SEQ ID NO: 2 and a VL comprising an amino acid sequence having at least 95%, 96%, 97%, 98%, 99%, or 100% sequence identity to the amino acid sequence of SEQ ID NO: 1. In certain embodiments, the antibody or fragment thereof comprises a VH encoded by a nucleic acid comprising a nucleic acid sequence having at least 95%, 96%, 97%, 98%, 99%, or 100% sequence identity to the nucleic acid sequence of SEQ ID NO: 8 and a VL region encoded by a nucleic acid comprising a nucleic acid sequence having at least 95%, 96%, 97%, 98%, 99%, or 100% sequence identity to the nucleic acid sequence of SEQ ID NO: 7.

In one embodiment, an antibody or an antibody fragment that binds to a B virus or a fragment thereof is provided, wherein the antibody comprises a VH comprising an amino acid sequence having at least 95%, 96%, 97%, 98%, 99%, or 100% sequence identity to the amino acid sequence of SEQ ID NO: 4 (or the VH region may be encoded by a nucleic acid comprising a nucleic acid sequence having at least 95%, 96%, 97%, 98%, 99%, or 100% sequence identity to the nucleic acid sequence of SEQ ID NO: 10). In certain embodiments, the antibody or fragment thereof further comprises a VL comprising an amino acid sequence having at least 95%, 96%, 97%, 98%, 99%, or 100% sequence identity to the amino acid sequence of SEQ ID NO: 3 (or the VL region may be encoded by a nucleic acid comprising a nucleic acid sequence having at least 95%, 96%, 97%, 98%, 99%, or 100% sequence identity to the nucleic acid sequence of SEQ ID NO: 9). In certain embodiments, the antibody or fragment thereof comprises a VH comprising an amino acid sequence having at least 95%, 96%, 97%, 98%, 99%, or 100% sequence identity to the amino acid sequence of SEQ ID NO: 4 and a VL comprising an amino acid sequence having at least 95%, 96%, 97%, 98%, 99%, or 100% sequence identity to the amino acid sequence of SEQ ID NO: 3. In certain embodiments, the antibody or fragment thereof comprises a VH encoded by a nucleic acid comprising a nucleic acid sequence having at least 95%, 96%, 97%, 98%, 99%, or 100% sequence identity to the nucleic acid sequence of SEQ ID NO: 10 and a VL region encoded by a nucleic acid comprising a nucleic acid sequence having at least 95%, 96%, 97%, 98%, 99%, or 100% sequence identity to the nucleic acid sequence of SEQ ID NO: 9.

In one embodiment, an antibody or an antibody fragment that binds to a B virus or a fragment thereof is provided, wherein the antibody comprises a VH comprising an amino acid sequence having at least 95%, 96%, 97%, 98%, 99%, or 100% sequence identity to the amino acid sequence of SEQ ID NO: 6 (or the VH region may be encoded by a nucleic acid comprising a nucleic acid sequence having at least 95%, 96%, 97%, 98%, 99%, or 100% sequence identity to the nucleic acid sequence of SEQ ID NO: 12). In certain embodiments, the antibody or fragment thereof further comprises a VL comprising an amino acid sequence having at least 95%, 96%, 97%, 98%, 99%, or 100% sequence identity to the amino acid sequence of SEQ ID NO: 5 (or the VL region may be encoded by a nucleic acid comprising a nucleic acid sequence having at least 95%, 96%, 97%, 98%, 99%, or 100% sequence identity to the nucleic acid sequence of SEQ ID NO: 11). In certain embodiments, the antibody or fragment thereof comprises a VH comprising an amino acid sequence having at least 95%, 96%, 97%, 98%, 99%, or 100% sequence identity to the amino acid sequence of SEQ ID NO: 6 and a VL comprising an amino acid sequence having at least 95%, 96%, 97%, 98%, 99%, or 100% sequence identity to the amino acid sequence of SEQ ID NO: 5. In certain embodiments, the antibody or fragment thereof comprises a VH encoded by a nucleic acid comprising a nucleic acid sequence having at least 95%, 96%, 97%, 98%, 99%, or 100% sequence identity to the nucleic acid sequence of SEQ ID NO: 12 and a VL region encoded by a nucleic acid comprising a nucleic acid sequence having at least 95%, 96%, 97%, 98%, 99%, or 100% sequence identity to the nucleic acid sequence of SEQ ID NO: 11.

In another aspect, the disclosure provides a method of treatment of a simplexvirus infection in a subject in need thereof, comprising administering any composition of the foregoing embodiments.

In some embodiments, the method further comprises a diagnostic assay to determine whether the subject has a simplexvirus infection using the antibodies. In other embodiments, the diagnostic assay can provide a differential diagnosis of the simplexvirus infection from the group comprising Rh-BV, Cyno-BV, PT-BV, Jap-BV, HSV-1 and/or HSV-2.

In some embodiments, the pharmaceutical composition is a vaccine. In other embodiments, the method of treatment comprises administering a vaccine.

In some embodiments, the method comprises treatment of Rh-BV, Jap-BV, PT-BV, and/or Cyno-BV infections with compositions according to any of claims 36-95 comprising:

one or more of anti-B virus antibody and/or fragment thereof as described herein;

B virus epitopes recognized by said MABs;

and/or other MABs specific to the B virus epitopes recognized by said MABs.

In other embodiments, the method comprises neutralizing the B virus by administration of a composition according to any of claims 36-95 comprising:

one or more of anti-B virus antibody and/or fragment thereof as described herein;

B virus epitopes recognized by said MABs; and/or

other MABs specific to the B virus epitopes recognized by said MABs.

In some embodiments, the disclosure provides a method of diagnosing a subject with a simplexvirus infection comprising use of one or more of anti-B virus antibody and/or fragment thereof as described herein. In certain embodiments, the simplexvirus infection is a B virus infection, a HSV-1 infection, and/or a HSV-2 infection.

In some embodiments, the method further comprises the steps of:

(i) taking a bodily fluid sample (e.g., blood, serum, plasma, urine, saliva, and/or CSF) from a subject;

(ii) determining whether the sample contains a simplexvirus; and

(iii) determining whether the subject is infected with a simplexvirus.

In other embodiments, the method is used to make a differential diagnosis among Rh-BV, Cyno-BV, PT-BV, Jap-BV, HSV-1, and/or HSV-2.

In certain embodiments, the step of determining whether the sample contains simplexviruses involves a hybridization assay.

In other embodiments, the method further comprises selecting a therapeutic composition and/or method based on the results of step (iii).

In certain embodiments, an antibody or an antibody fragment that binds to a B virus or a fragment thereof is provided, wherein the antibody or fragment thereof binds to an epitope within a fragment of a B virus. In certain embodiments, an antibody or an antibody fragment that binds to a B virus or a fragment thereof is provided, wherein the antibody or fragment thereof binds to an epitope within a fragment of a B virus comprising any of the amino acid sequences provided in Table 7 herein (e.g., SEQ ID NOs: 13-85).

In certain embodiments, the anti-B virus antibody is a monoclonal antibody. In certain embodiments, the anti-B virus antibody is humanized. In certain embodiments, the anti-B virus antibody is a human antibody. In certain embodiments, at least a portion of the framework sequence of the anti-B virus antibody is a human consensus framework sequence. In one embodiment, the antibody is an antibody fragment selected from a Fab, Fab′-SH, Fv, scFv, or (Fab′)₂ fragment.

In one aspect, a nucleic acid encoding any of the above anti-B virus antibodies or fragments thereof is provided. In one embodiment, a vector comprising the nucleic acid is provided. In one embodiment, the vector is an expression vector. In one embodiment, a host cell comprising the vector is provided. In one embodiment, the host cell is eukaryotic. In another embodiment, the host cell is mammalian. In yet another embodiment, the host cell is prokaryotic. In one embodiment, a method of making an anti-B virus antibody or fragment thereof is provided, wherein the method comprises culturing the host cell under conditions suitable for expression of the nucleic acid encoding the antibody, and isolating the antibody. In certain embodiment, the method further comprises recovering the anti-B virus antibody or fragment thereof from the host cell. In certain embodiments, a composition comprising any of the anti-B virus antibodies or fragments thereof described herein is provided. In one embodiment, the composition further comprises a pharmaceutically acceptable carrier.

In one aspect, provided herein is a pharmaceutical composition comprising an anti-B virus antibody or fragment thereof. In certain embodiments, the composition is suitable for subcutaneous administration. In certain embodiments, the composition is suitable for intravenous administration. Any anti-B virus antibodies known in the art or described herein may be formulated into the composition.

Any embodiment described herein or any combination thereof applies to any and all anti-B antibodies or fragments thereof, methods and uses of the disclosure described herein.

It is also intended that the isolated antibodies or fragments thereof that interact with B viruses of the present application may be a glycosylated binding protein wherein the antibody or antigen-binding portion thereof comprises one or more carbohydrate residues. Nascent in vivo protein production may undergo further processing, known as post-translational modification. In particular, sugar (glycosyl) residues may be added enzymatically, a process known as glycosylation. The resulting proteins bearing covalently linked oligosaccharide side chains are known as glycosylated proteins or glycoproteins. Protein glycosylation depends on the amino acid sequence of the protein of interest, as well as the host cell in which the protein is expressed. Different organisms may produce different glycosylation enzymes (e.g., glycosyltransferases and glycosidases), and have different substrates (nucleotide sugars) available. Due to such factors, protein glycosylation pattern, and composition of glycosyl residues, may differ depending on the host system in which the particular protein is expressed. Glycosyl residues useful in the disclosure may include, but are not limited to, glucose, galactose, mannose, fucose, n-acetylglucosamine and sialic acid.

The antibodies or fragments thereof of the present application comprise a heavy chain constant region, such as an IgG1, IgG2, IgG3, IgG4, IgA, IgE, IgM or IgD constant region. Furthermore, the antibody can comprise a light chain constant region, either a kappa light chain constant region or a lambda light chain constant region. Particularly, the antibody comprises a kappa light chain constant region. Alternatively, the antibody portion can be, for example, a Fab fragment or a single chain Fv fragment. Replacements of amino acid residues in the Fc portion to alter antibody effector function are known in the art (U.S. Pat. Nos. 5,648,260; 5,624,821). The Fc portion of an antibody mediates several important effector functions, e.g., cytokine induction, ADCC, phagocytosis, complement dependent cytotoxicity (CDC) and half-life/clearance rate of antibody and antigen-antibody complexes. In some cases these effector functions are desirable for therapeutic antibodies but in other cases might be unnecessary or even deleterious, depending on the therapeutic objectives. Certain human IgG isotypes, particularly IgG1 and IgG3, mediate ADCC and CDC via binding to Fcγ Rs and complement C1q, respectively. Neonatal Fc receptors (FcRn) are the critical components determining the circulating half-life of antibodies. In still another embodiment at least one amino acid residue is replaced in the constant region of the antibody, for example the Fc region of the antibody, such that effector functions of the antibody are altered.

These and other objects, features and advantages of the disclosure will become more apparent upon reading the following specification in conjunction with the accompanying description and claims.

### DETAILED DESCRIPTION OF THE INVENTION

As specified in the Background Section, there is a great need in the art to identify reagents specific for certain B virus subtypes and use this understanding to develop novel compositions and assays for differential diagnosis of various B viruses in monkeys and humans, treatment of said B viruses, and identification of peptides enabling said differential diagnosis. The disclosure satisfies this and other needs. Embodiments of the disclosure relates generally to B virus epitopes and monoclonal antibodies (MABs) that can distinguish between different B viruses specific to different hosts and can further neutralize said viruses; compositions containing the epitopes and MABs and nucleic acids encoding the epitopes and MABs; and specific B virus epitopes and MABs can be used in diagnostic assays or as sources for vaccines or other therapeutic interventions.

### Definitions

To facilitate an understanding of the principles and features of the various embodiments of the disclosure, various illustrative embodiments are explained below. Although exemplary embodiments of the disclosure are explained in detail, it is to be understood that other embodiments are contemplated. Accordingly, it is not intended that the disclosure is limited in its scope to the details of construction and arrangement of components set forth in the following description or examples. The disclosure is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the disclosure as many comparable parameters, sizes, ranges, and/or values may be implemented. The terms “first,” “second,” and the like, “primary,” “secondary,” and the like, do not denote any order, quantity, or importance, but rather are used to distinguish one element from another.

It is noted that terms like “specifically,” “preferably,” “typically,” “generally,” and “often” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the disclosure. It is also noted that terms like “substantially” and “about” are utilized herein to represent the inherent degree of uncertainty that may be attributed to any quantitative comparison, value, measurement, or other representation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “50 mm” is intended to mean “about 50 mm.”

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the disclosure are intended to be illustrative and not restrictive. Many suitable materials that would perform the same or a similar function as the materials described herein are intended to be embraced within the scope of the disclosure. Such other materials not described herein can include, but are not limited to, materials that are developed after the time of the development of the disclosure, for example. Any dimensions listed in the various drawings are for illustrative purposes only and are not intended to be limiting. Other dimensions and proportions are contemplated and intended to be included within the scope of the disclosure.

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

As used herein, the term “sample” refers to anything which may contain an analyte for which an analyte assay is desired. The sample may be a biological sample, such as a biological fluid or a biological tissue. Examples of biological fluids include blood, serum, plasma, saliva, sputum, ocular lens fluid, sweat, urine, milk, ascites fluid, mucous, synovial fluid, peritoneal fluid, transdermal exudates, pharyngeal exudates, bronchoalveolar lavage, tracheal aspirations, cerebrospinal fluid, semen, cervical mucus, vaginal or urethral secretions, amniotic fluid, and the like. Biological tissues comprise an aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). The sample can be used as obtained directly from the source or following a pretreatment so as to modify its character.

As used herein, the term “combination” of a B virus epitope and at least a second pharmaceutically active ingredient means at least two, but any desired combination of compounds can be delivered simultaneously or sequentially (e.g., within a 24 hour period). It is contemplated that when used to treat various diseases, the compositions and methods of the disclosure can be utilized with other therapeutic methods/agents suitable for the same or similar diseases. Such other therapeutic methods/agents can be co-administered (simultaneously or sequentially) to generate additive or synergistic effects. Suitable therapeutically effective dosages for each agent may be lowered due to the additive action or synergy.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

A “therapeutically effective amount” means the amount of a compound (e.g., a B virus antibody or fragment thereof, or a B virus epitope) that, when administered to a subject for treating (e.g., preventing or ameliorating) a state, disorder or condition, is sufficient to effect such treatment. The “therapeutically effective amount” will vary depending on the compound administered as well as the disease and its severity and the age, weight, physical condition and responsiveness of the mammal to be treated.

The phrase “pharmaceutically acceptable”, as used in connection with compositions of the disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

As used herein, the term “combination” of a composition according to the disclosure and at least a second pharmaceutically active ingredient means at least two, but any desired combination of compounds can be delivered simultaneously or sequentially (e.g., within a 24 hour period).

Within the meaning of the disclosure, the term “conjoint administration” is used to refer to administration of a composition according to the disclosure and another therapeutic agent simultaneously in one composition, or simultaneously in different compositions, or sequentially (preferably, within a 24 hour period).

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An antibody broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art, nonlimiting embodiments of which are discussed below. An antibody is said to be “capable of binding” a molecule if it is capable of specifically reacting with the molecule to thereby bind the molecule to the antibody.

An “antigen-binding portion” or “antigen-binding fragment” of an antibody (or simply “antibody portion” or “antibody fragment”) refers to a molecule other than an intact antibody that comprises a portion of an intact antibody that binds the antigen to which the intact antibody binds (e.g., one or more fragments of an antibody that retain the ability to specifically bind to an antigen). Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂fragment that has two antigen-combining sites and is still capable of cross-linking antigen. It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)₂fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv). Such single chain antibodies are also intended to be encompassed within the term “antigen-binding portion” of an antibody. Other forms of single chain antibodies, such as diabodies are also encompassed.

The term “chimeric” antibody refers to an antibody in which a portion of the heavy and/or light chain is derived from a particular source or species, and at least one other portion of the heavy and/or light chain, including the remainder thereof, is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent and/or bispecific. Diabodies are described more fully in, for example, EP 404,097; WO 1993/01161; Hudson et al., Nat. Med. 9: 129-134 (2003); and Hollinger et al., Proc. Natl. Acad. Sci. USA 90: 6444-6448 (1993). Triabodies and tetrabodies are also described in Hudson et al., Nat. Med. 9: 129-134 (2003).

The term “epitope” or “antigenic determinant” includes any polypeptide determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules.

The “Fab” fragment contains the heavy- and light-chain variable domains and also contains the constant domain of the light chain and the first constant domain (CHI) of the heavy chain. Fab′ fragments differ from Fab fragments by the addition of a few residues at the carboxy terminus of the heavy chain CHI domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “Fc region” herein is used to define a C-terminal region of an immunoglobulin heavy chain that contains at least a portion of the constant region. The term includes native sequence Fc regions and variant Fc regions.

“Framework” or “FR” refers to variable domain residues other than complementarity determining region (CDR) residues. The FR of a variable domain generally consists of four FR domains: FR1, FR2, FR3, and FR4. Accordingly, the CDR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

A “humanized” antibody refers to an antibody comprising heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. A humanized antibody may thus comprise amino acid residues from non-human CDRs and amino acid residues from human FRs. In certain embodiments, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. A humanized antibody comprises substantially all of at least one, and typically two, variable domains (Fab, Fab′, F(ab′)2, FabC, Fv) in which all or substantially all of the CDR regions correspond to those of a non-human immunoglobulin (i.e., donor antibody) and all or substantially all of the framework regions are those of a human immunoglobulin consensus sequence. Particularly, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain. The humanized antibody can be selected from any class of immunoglobulins, including IgY, IgM, IgG, IgD, IgA and IgE, and any isotype, including without limitation IgA1, IgA2, IgG1, IgG2, IgG3 and IgG4. The humanized antibody may comprise sequences from more than one class or isotype, and particular constant domains may be selected to optimize desired effector functions using techniques well-known in the art. The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In a particular embodiment, such mutations, however, will not be extensive. Usually, at least 50, 55, 60, 65, 70, 75 or 80%, particularly at least 85%, more particularly at least 90%, and in particular at least 95% of the humanized antibody residues will correspond to those of the parental FR and CDR sequences.

The term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

An “immunoconjugate” is an antibody conjugated to one or more heterologous molecule(s), including but not limited to a cytotoxic agent.

An “isolated” antibody is one which has been separated from a component of its natural environment. In some embodiments, an antibody is purified to greater than 95% or 99% purity as determined by, for example and not limitation, electrophoretic methods (e.g., SDS-PAGE, isoelectric focusing (IEF), capillary electrophoresis) or chromatographic methods (e.g., ion exchange or reverse phase HPLC). An isolated antibody is substantially free of other antibodies having different antigenic specificities (e.g., an isolated antibody that specifically binds a B virus is substantially free of antibodies that specifically bind antigens other than a B virus). An isolated antibody that specifically binds a B virus may, however, have cross-reactivity to other antigens, such as other B viruses and/or simplexviruses.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-B virus antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody,” as used herein, refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

The term “polynucleotide” as referred to herein means a polymeric form of two or more nucleotides, either ribonucleotides or deoxynucleotides or a modified form of either type of nucleotide. The term includes single and double stranded forms of DNA but particularly is double-stranded DNA.

The term “isolated polynucleotide” as used herein shall mean a polynucleotide (e.g., of genomic, cDNA, or synthetic origin, or some combination thereof) that, by virtue of its origin, the “isolated polynucleotide”: is not associated with all or a portion of a polynucleotide with which the “isolated polynucleotide” is found in nature; is operably linked to a polynucleotide that it is not linked to in nature; or does not occur in nature as part of a larger sequence.

The term “polypeptide” as used herein, refers to any polymeric chain of amino acids. The terms “peptide” and “protein” are used interchangeably with the term polypeptide and also refer to a polymeric chain of amino acids. The term “polypeptide” encompasses native or artificial proteins, protein fragments and polypeptide analogs of a protein sequence. A polypeptide may be monomeric or polymeric.

The term “isolated protein” or “isolated polypeptide” is a protein or polypeptide that by virtue of its origin or source of derivation is not associated with naturally associated components that accompany it in its native state; is substantially free of other proteins from the same species; is expressed by a cell from a different species; or does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

In accordance with the disclosure there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds.(1985); Transcription and Translation (B. D. Hames & S. J. Higgins, eds. (1984); Animal Cell Culture (R I. Freshney, ed. (1986); Immobilized Cells and Enzymes (IRL Press, (1986); B. Perbal, A Practical Guide To Molecular Cloning (1984); F. M. Ausubel et al. (eds.), Current Protocols in Molecular Biology, John Wiley & Sons, Inc. (1994); among others.

### Therapeutic Compositions and Methods

The disclosure provides the use of monoclonal antibodies (MABs) as described in Table 1 herein as pharmaceutical compositions and can be optionally combined with other compositions of the disclosure or other therapeutic molecules and/or treatments for simplexviruses including B viruses. In certain embodiments, a MAB is used before, during, and/or after such other therapeutic molecules and/or treatments. The disclosure also provides the use of B virus epitopes as described in Table 7 herein (e.g., SEQ ID NOs: 13-85) as pharmaceutical compositions and can be optionally combined with other compositions of the disclosure or other therapeutic molecules and/or treatments for simplexviruses including B viruses. In certain embodiments, a B virus epitope (e.g., SEQ ID NOs: 13-85) is used before, during, and/or after such other therapeutic molecules and/or treatments. In other embodiments, a homologous variant of a B virus epitope (e.g., an amino acid sequence having at least 95%, 96%, 97%, 98%, 99%, or 100% sequence identity to any of SEQ ID NOs: 13-85) is provided as a pharmaceutical composition which can be optionally combined with other compositions of the disclosure or other therapeutic molecules and/or treatments for simplexviruses including B viruses, which may be used before, during, and/or after such other therapeutic molecules and/or treatments.

The present disclosure provides binding molecules, in particular antibodies, which bind specifically and/or differentially to B viruses; representative anti-B virus antibodies of the disclosure may comprise at least one of the antibody variable region amino acid sequences shown in SEQ ID NOs: 1-6 (and/or encoded by the nucleic acid sequences shown in SEQ ID NOs: 7-12, or individual CDRs thereof or related CDR sequences, as specified in more detail below.

Specifically the present disclosure provides antibodies that bind specifically and/or differentially to B viruses, more specifically monoclonal antibodies. The present disclosure also provides B virus-binding fragments that are at least 95%, 96%, 97%, 98%, 99%, or 100% identical to any of SEQ ID NOs: 1-6 or 7-12.

Included in the present disclosure are anti-B virus antibodies and fragments thereof that bind specifically and/or differentially to B viruses and comprise a light chain variable region having an amino acid sequence that is at least 95%, 96%, 97%, 98%, 99%, or 100% identical to any of SEQ ID NOs: 1, 3 and 5, or is a B virus-binding fragment or a homologous variant thereof, of an antibody comprised in said sequences. Alternatively, the anti-B virus antibodies and fragments thereof may be encoded by a nucleic acid having a nucleic acid sequence that is at least 95%, 96%, 97%, 98%, 99%, or 100% identical to any of SEQ ID NOs: 7, 9 and 11.

Also included are anti-B virus antibodies and fragments thereof that bind specifically and/or differentially to B viruses and comprise a heavy chain variable region having an amino acid sequence that is at least 95%, 96%, 97%, 98%, 99%, or 100% identical to any of SEQ ID NOs: 2, 4, and 6, or is a B virus-binding fragment or a homologous variant thereof, of an antibody comprised in said sequences. Alternatively, the anti-B virus antibodies and fragments thereof may be encoded by a nucleic acid having a nucleic acid sequence that is at least 95%, 96%, 97%, 98%, 99%, or 100% identical to any of SEQ ID NOs: 8, 10 and 12.

The anti-B virus antibodies of the disclosure include an anti-B virus antibody or fragment thereof or a B virus-binding fragment or homologous variant thereof as described above, which is selected from the group consisting of a chimeric antibody, a CDR-grafted or humanized antibody, a single chain antibody, a fusion protein, and a human antibody.

In certain embodiments, an antibody or an antibody fragment that binds to a B virus or a fragment thereof is provided, wherein the antibody or fragment thereof comprises a variable domain comprising a heavy chain (VH) comprising the amino acid sequence of SEQ ID NO: 2, 4, or 6 and a light chain (VL) comprising the amino acid sequence of SEQ ID NO: 1, 3, or 5. Alternatively, the VH region may be encoded by a nucleic acid comprising the nucleic acid sequence of SEQ ID NO: 8, 10, or 12 and a light chain (VL) comprising the amino acid sequence of SEQ ID NO: 7, 9, or 11.

In certain embodiments, an antibody or an antibody fragment that binds to a B virus or a fragment thereof is provided, wherein the antibody or fragment thereof comprises (a) a VH sequence having at least 95%, 96%, 97%, 98%, 99%, or 100% sequence identity to the amino acid sequence of SEQ ID NO: 2, 4, or 6; or (b) a VL sequence having at least 9595%, 96%, 97%, 98%, 99%, or 100% sequence identity to the amino acid sequence of SEQ ID NO: 1, 3, or 5.

In other embodiments, an an antibody or an antibody fragment that binds to a B virus or a fragment thereof is provided, wherein the antibody or fragment thereof comprises (a) a VH sequence encoded by a nucleic acid having at least 95%, 96%, 97%, 98%, 99%, or 100% sequence identity to the nucleic acid sequence of SEQ ID NO: 8, 10, or 12; or (b) a VL sequence having at least 95%, 96%, 97%, 98%, 99%, or 100% sequence identity to the amino acid sequence of SEQ ID NO: 7, 9, or 11.

In one embodiment, an antibody or an antibody fragment that binds to a B virus or a fragment thereof is provided, wherein the antibody comprises a VH comprising the amino acid sequence of SEQ ID NO: 2 (or the VH region may be encoded by a nucleic acid comprising the nucleic acid sequence of SEQ ID NO: 8). In certain embodiments, the antibody or fragment thereof further comprises a VL comprising the amino acid sequence of SEQ ID NO: 1 (or the VL region may be encoded by a nucleic acid comprising the nucleic acid sequence of SEQ ID NO: 7). In certain embodiments, the antibody or fragment thereof comprises a VH comprising the amino acid sequence of SEQ ID NO: 2 and a VL comprising the amino acid sequence of SEQ ID NO: 1. In certain embodiments, the antibody or fragment thereof comprises a VH encoded by a nucleic acid comprising the nucleic acid sequence of SEQ ID NO: 8 and a VL region encoded by a nucleic acid comprising the nucleic acid sequence of SEQ ID NO: 7.

In one embodiment, an antibody or an antibody fragment that binds to a B virus or a fragment thereof is provided, wherein the antibody comprises a VH comprising the amino acid sequence of SEQ ID NO: 4 (or the VH region may be encoded by a nucleic acid comprising the nucleic acid sequence of SEQ ID NO: 10). In certain embodiments, the antibody or fragment thereof further comprises a VL comprising the amino acid sequence of SEQ ID NO: 3 (or the VL region may be encoded by a nucleic acid comprising the nucleic acid sequence of SEQ ID NO: 9). In certain embodiments, the antibody or fragment thereof comprises a VH comprising the amino acid sequence of SEQ ID NO: 4 and a VL comprising the amino acid sequence of SEQ ID NO: 3. In certain embodiments, the antibody or fragment thereof comprises a VH encoded by a nucleic acid comprising the nucleic acid sequence of SEQ ID NO: 10 and a VL region encoded by a nucleic acid comprising the nucleic acid sequence of SEQ ID NO: 9.

In one embodiment, an antibody or an antibody fragment that binds to a B virus or a fragment thereof is provided, wherein the antibody comprises a VH comprising the amino acid sequence of SEQ ID NO: 6 (or the VH region may be encoded by a nucleic acid comprising the nucleic acid sequence of SEQ ID NO: 12). In certain embodiments, the antibody or fragment thereof further comprises a VL comprising the amino acid sequence of SEQ ID NO: 5 (or the VL region may be encoded by a nucleic acid comprising the nucleic acid sequence of SEQ ID NO: 11). In certain embodiments, the antibody or fragment thereof comprises a VH comprising the amino acid sequence of SEQ ID NO: 6 and a VL comprising the amino acid sequence of SEQ ID NO: 5. In certain embodiments, the antibody or fragment thereof comprises a VH encoded by a nucleic acid comprising the nucleic acid sequence of SEQ ID NO: 12 and a VL region encoded by a nucleic acid comprising the nucleic acid sequence of SEQ ID NO: 11.

In one embodiment, an antibody or an antibody fragment that binds to a B virus or a fragment thereof is provided, wherein the antibody comprises a VH comprising an amino acid sequence having at least 95%, 96%, 97%, 98%, 99%, or 100% sequence identity to the amino acid sequence of SEQ ID NO: 2 (or the VH region may be encoded by a nucleic acid comprising a nucleic acid sequence having at least 95%, 96%, 97%, 98%, 99%, or 100% sequence identity to the nucleic acid sequence of SEQ ID NO: 8). In certain embodiments, the antibody or fragment thereof further comprises a VL comprising an amino acid sequence having at least 95%, 96%, 97%, 98%, 99%, or 100% sequence identity to the amino acid sequence of SEQ ID NO: 1 (or the VL region may be encoded by a nucleic acid comprising a nucleic acid sequence having at least 95%, 96%, 97%, 98%, 99%, or 100% sequence identity to the nucleic acid sequence of SEQ ID NO: 7). In certain embodiments, the antibody or fragment thereof comprises a VH comprising an amino acid sequence having at least 95%, 96%, 97%, 98%, 99%, or 100% sequence identity to the amino acid sequence of SEQ ID NO: 2 and a VL comprising an amino acid sequence having at least 95%, 96%, 97%, 98%, 99%, or 100% sequence identity to the amino acid sequence of SEQ ID NO: 1. In certain embodiments, the antibody or fragment thereof comprises a VH encoded by a nucleic acid comprising a nucleic acid sequence having at least 95%, 96%, 97%, 98%, 99%, or 100% sequence identity to the nucleic acid sequence of SEQ ID NO: 8 and a VL region encoded by a nucleic acid comprising a nucleic acid sequence having at least 95%, 96%, 97%, 98%, 99%, or 100% sequence identity to the nucleic acid sequence of SEQ ID NO: 7.

In one embodiment, an antibody or an antibody fragment that binds to a B virus or a fragment thereof is provided, wherein the antibody comprises a VH comprising an amino acid sequence having at least 95%, 96%, 97%, 98%, 99%, or 100% sequence identity to the amino acid sequence of SEQ ID NO: 4 (or the VH region may be encoded by a nucleic acid comprising a nucleic acid sequence having at least 95%, 96%, 97%, 98%, 99%, or 100% sequence identity to the nucleic acid sequence of SEQ ID NO: 10). In certain embodiments, the antibody or fragment thereof further comprises a VL comprising an amino acid sequence having at least 95%, 96%, 97%, 98%, 99%, or 100% sequence identity to the amino acid sequence of SEQ ID NO: 3 (or the VL region may be encoded by a nucleic acid comprising a nucleic acid sequence having at least 95%, 96%, 97%, 98%, 99%, or 100% sequence identity to the nucleic acid sequence of SEQ ID NO: 9). In certain embodiments, the antibody or fragment thereof comprises a VH comprising an amino acid sequence having at least 95%, 96%, 97%, 98%, 99%, or 100% sequence identity to the amino acid sequence of SEQ ID NO: 4 and a VL comprising an amino acid sequence having at least 95%, 96%, 97%, 98%, 99%, or 100% sequence identity to the amino acid sequence of SEQ ID NO: 3. In certain embodiments, the antibody or fragment thereof comprises a VH encoded by a nucleic acid comprising a nucleic acid sequence having at least 95%, 96%, 97%, 98%, 99%, or 100% sequence identity to the nucleic acid sequence of SEQ ID NO: 10 and a VL region encoded by a nucleic acid comprising a nucleic acid sequence having at least 95%, 96%, 97%, 98%, 99%, or 100% sequence identity to the nucleic acid sequence of SEQ ID NO: 9.

In one embodiment, an antibody or an antibody fragment that binds to a B virus or a fragment thereof is provided, wherein the antibody comprises a VH comprising an amino acid sequence having at least 95%, 96%, 97%, 98%, 99%, or 100% sequence identity to the amino acid sequence of SEQ ID NO: 6 (or the VH region may be encoded by a nucleic acid comprising a nucleic acid sequence having at least 95%, 96%, 97%, 98%, 99%, or 100% sequence identity to the nucleic acid sequence of SEQ ID NO: 12). In certain embodiments, the antibody or fragment thereof further comprises a VL comprising an amino acid sequence having at least 95%, 96%, 97%, 98%, 99%, or 100% sequence identity to the amino acid sequence of SEQ ID NO: 5 (or the VL region may be encoded by a nucleic acid comprising a nucleic acid sequence having at least 95%, 96%, 97%, 98%, 99%, or 100% sequence identity to the nucleic acid sequence of SEQ ID NO: 11). In certain embodiments, the antibody or fragment thereof comprises a VH comprising an amino acid sequence having at least 95%, 96%, 97%, 98%, 99%, or 100% sequence identity to the amino acid sequence of SEQ ID NO: 6 and a VL comprising an amino acid sequence having at least 95%, 96%, 97%, 98%, 99%, or 100% sequence identity to the amino acid sequence of SEQ ID NO: 5. In certain embodiments, the antibody or fragment thereof comprises a VH encoded by a nucleic acid comprising a nucleic acid sequence having at least 95%, 96%, 97%, 98%, 99%, or 100% sequence identity to the nucleic acid sequence of SEQ ID NO: 12 and a VL region encoded by a nucleic acid comprising a nucleic acid sequence having at least 95%, 96%, 97%, 98%, 99%, or 100% sequence identity to the nucleic acid sequence of SEQ ID NO: 11.

In certain embodiments, an antibody or an antibody fragment that binds to a B virus or a fragment thereof is provided, wherein the antibody or fragment thereof binds to an epitope within a fragment of a B virus. In certain embodiments, an antibody or an antibody fragment that binds to a B virus or a fragment thereof is provided, wherein the antibody or fragment thereof binds to an epitope within a fragment of a B virus comprising any of the amino acid sequences provided in Table 7 herein.

In certain embodiments, the anti-B virus antibody is a monoclonal antibody. In certain embodiments, the anti-B virus antibody is humanized. In certain embodiments, the anti-B virus antibody is a human antibody. In certain embodiments, at least a portion of the framework sequence of the anti-B virus antibody is a human consensus framework sequence. In one embodiment, the antibody is an antibody fragment selected from a Fab, Fab′-SH, Fv, scFv, or (Fab′)₂ fragment.

In another aspect, an anti-B virus antibody or fragment thereof is provided, wherein the antibody or fragment comprises a VH as in any of the embodiments provided above, and a VL as in any of the embodiments provided above. In another aspect, an anti-B virus antibody or fragment thereof is provided, wherein the antibody or fragment comprises a VH that is encoded by a nucleic acid as in any of the embodiments provided above, and a VL that is encoded by a nucleic acid as in any of the embodiments provided above.

In any of the above embodiments, the anti-B virus antibody or fragment thereof is selected from the group consisting of an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, a dual variable domain immunoglobulin, and a bispecific antibody.

In any of the above embodiments, the anti-B virus antibody or fragment thereof comprises a heavy chain immunoglobulin constant domain selected from the group consisting of a human IgM constant domain, a human IgG1 constant domain, a human IgG2 constant domain, a human IgG3 constant domain, a human IgG4 constant domain, a human IgE constant domain, a human IgD constant domain, a human IgA1 constant domain, a human IgA2 constant domain, a human IgY constant domain, and corresponding mutated domains.

In any of the above embodiments, the anti-B virus antibody or fragment thereof is present as an immunoconjugate, further comprising an agent selected from the group consisting of an immunoadhesion molecule, an imaging agent (such as for example and not limitation, a radiolabel (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶HO, and ¹⁵³Sm), an enzyme, a fluorescent label, a luminescent label, a bioluminescent label, a magnetic label, and biotin), a therapeutic agent, and a cytotoxic agent. Exemplary therapeutic or cytotoxic agents include an anti-metabolite, an alkylating agent, an antibiotic, a growth factor, a cytokine, an anti-angiogenic agent, an anti-mitotic agent, an anthracycline, toxin, and an apoptotic agent.

In one aspect, a nucleic acid encoding any of the above anti-B virus antibodies or fragments thereof is provided. In one embodiment, a vector comprising the nucleic acid is provided. In one embodiment, the vector is an expression vector. In one embodiment, a host cell comprising the vector is provided. In another embodiment, a host cell transformed with the vector is provided. In one embodiment, the host cell is eukaryotic (such as for example and not limitation, protist cell, animal cell, plant cell, fungal cell, mammalian cell, avian cell, insect cell, HEK Cells, CHO cells, COS cells and yeast cells). In another embodiment, the host cell is mammalian. In yet another embodiment, the host cell is prokaryotic.

In one aspect, the disclosure provides a hybridoma cell line that produces an anti-B virus antibody or antigen-binding fragment thereof. In certain embodiments, the hybridoma is selected from the group consisting of mouse, human, rat, sheep, pig, cattle, goat, and horse hybridoma. In certain embodiments, the hybridoma cell line produces an anti-B virus antibody or antigen-binding fragment thereof (e.g., a monoclonal antibody), which specifically binds to at least one epitope of a B virus.

In one embodiment, a method of making an anti-B virus antibody or fragment thereof is provided, wherein the method comprises culturing the host cell under conditions suitable for expression of the nucleic acid encoding the antibody, and isolating the antibody. In certain embodiment, the method further comprises recovering the anti-B virus antibody or fragment thereof from the host cell.

In certain embodiments, a composition comprising any of the anti-B virus antibodies or fragments thereof described herein is provided. In one embodiment, the composition further comprises a pharmaceutically acceptable carrier.

In one aspect, provided herein is a pharmaceutical composition comprising an anti-B virus antibody or fragment thereof. In certain embodiments, the composition is suitable for subcutaneous administration. In certain embodiments, the composition is suitable for intravenous administration. Any anti-B virus antibodies known in the art or described herein may be formulated into the composition. In certain embodiments, the composition comprises a pharmaceutically acceptable carrier, such as for example and not limitation, a polymeric carrier. In certain embodiments, the composition may further comprise albumin, sucrose, trehalose, lactitol, gelatin, hydroxypropyl-0-cyclodextrin, methoxypolyethylene glycol and polyethylene glycol. In certain embodiments, the composition may further comprise an adjuvant. In certain embodiments, the composition may further comprise an additional agent, such as for example and not limitation, a therapeutic agent, imaging agent, cytotoxic agent, angiogenesis inhibitors; kinase inhibitors; co-stimulation molecule blockers; adhesion molecule blockers; anti-cytokine antibody or functional fragment thereof; methotrexate; cyclosporin; rapamycin; FK506; detectable label or reporter; a TNF antagonist; an antirheumatic; a muscle relaxant, a narcotic, a non-steroid anti-inflammatory drug (NSAID), an analgesic, an anesthetic, a sedative, a local anesthetic, a neuromuscular blocker, an antimicrobial, an antipsoriatic, a corticosteriod, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, and a cytokine antagonist. Further examples are: Dimebon, anti-Aβ-antibodies, beta-secretase inhibitors, tau-modulators, cognition enhancers like e.g. 5-HT6 antagonists, cholesterinase inhibitor (e.g., tactrine, donepezil, rivastigmine or galantamine), a partial NMDA receptor blocker (e.g., memantine), a glycosaminoglycan mimetic (e.g., Alzhemed), an inhibitor or allosteric modulator of gamma secretase (e.g., R-flurbiprofen), a luteinizing hormone blockade gonadotropin releasing hormone agonist (e.g., leuprorelin), a serotinin 5-HT1A receptor antagonist, a chelatin agent, a neuronal selective L-type calcium channel blocker, an immunomodulator, an amyloid fibrillogenesis inhibitor or amyloid protein deposition inhibitor (e.g., M266), another antibody (e.g., bapineuzumab), a 5-HT1a receptor antagonist, a PDE4 inhibitor, a histamine agonist, a receptor protein for advanced glycation end products, a PARP stimulator, a serotonin 6 receptor antagonist, a 5-HT4 receptor agonist, a human steroid, a glucose uptake stimulant which enhanced neuronal metabolism, a selective CB1 antagonist, a partial agonist at benzodiazepine receptors, an amyloid beta production antagonist or inhibitor, an amyloid beta deposition inhibitor, a NNR alpha-7 partial antagonist, a therapeutic targeting PDE4, a RNA translation inhibitor, a muscarinic agonist, a nerve growth factor receptor agonist, a NGF receptor agonist and a gene therapy modulator. In certain embodiments, the composition may comprise a further agent comprising an antiviral agent, such as for example and not limitation, ganciclovir and/or acyclovir, as well as derivatives, analogs, prodrugs and modifications thereof. Other antiviral agents not yet known may be used in combination with the pharmaceutical compositions of the disclosure.

Any embodiment described herein or any combination thereof applies to any and all anti-B antibodies or fragments thereof, methods and uses of the disclosure described herein.

It is also intended that the isolated antibodies or fragments thereof that interact with B viruses of the present application may be a glycosylated binding protein wherein the antibody or antigen-binding portion thereof comprises one or more carbohydrate residues. Nascent in vivo protein production may undergo further processing, known as post-translational modification. In particular, sugar (glycosyl) residues may be added enzymatically, a process known as glycosylation. The resulting proteins bearing covalently linked oligosaccharide side chains are known as glycosylated proteins or glycoproteins. Protein glycosylation depends on the amino acid sequence of the protein of interest, as well as the host cell in which the protein is expressed. Different organisms may produce different glycosylation enzymes (e.g., glycosyltransferases and glycosidases), and have different substrates (nucleotide sugars) available. Due to such factors, protein glycosylation pattern, and composition of glycosyl residues, may differ depending on the host system in which the particular protein is expressed. Glycosyl residues useful in the disclosure may include, but are not limited to, glucose, galactose, mannose, fucose, n-acetylglucosamine and sialic acid.

The antibodies or fragments thereof of the present application comprise a heavy chain constant region, such as an IgG1, IgG2, IgG3, IgG4, IgA, IgE, IgM or IgD constant region. Furthermore, the antibody can comprise a light chain constant region, either a kappa light chain constant region or a lambda light chain constant region. Particularly, the antibody comprises a kappa light chain constant region. Alternatively, the antibody portion can be, for example, a Fab fragment or a single chain Fv fragment. Replacements of amino acid residues in the Fc portion to alter antibody effector function are known in the art (U.S. Pat. Nos. 5,648,260; 5,624,821). The Fc portion of an antibody mediates several important effector functions, e.g., cytokine induction, ADCC, phagocytosis, complement dependent cytotoxicity (CDC) and half-life/clearance rate of antibody and antigen-antibody complexes. In some cases these effector functions are desirable for therapeutic antibodies but in other cases might be unnecessary or even deleterious, depending on the therapeutic objectives. Certain human IgG isotypes, particularly IgG1 and IgG3, mediate ADCC and CDC via binding to Fcγ Rs and complement C1q, respectively. Neonatal Fc receptors (FcRn) are the critical components determining the circulating half-life of antibodies. In still another embodiment at least one amino acid residue is replaced in the constant region of the antibody, for example the Fc region of the antibody, such that effector functions of the antibody are altered.

In some embodiments of the disclosure, at least one MAB and/or B virus epitope is formulated into a suitable pharmaceutical preparation such as, e.g., solution, suspension, tablet, dispersible tablet, pill, capsule, powder, sustained release formulation or elixir, for oral administration; sterile solution or suspension for parenteral administration; powdered or liquid spray, nose drops, a gel or ointment for intranasal administration; powdered or liquid spray for administration by inhalation; films for sublingual administration; patch for transdermal administration, etc. A MAB can be formulated into pharmaceutical compositions using any of the techniques and procedures known in the art (see, e.g., Ansel Introduction to Pharmaceutical Dosage Forms, Fourth Edition 1985, 126).

In some embodiments, the compositions contain effective concentrations of one or more MABs and/or B virus epitopes or pharmaceutically acceptable derivatives thereof is (are) mixed with a suitable pharmaceutical carrier or vehicle.

The amount of the MABs and/or B virus epitopes administered and the regimen of administration depends on absorption, inactivation and excretion rates of the active agent, the physicochemical characteristics of the agent, the severity of the condition to be alleviated, the age, condition, body weight, sex and diet of the patient, the disease state, other medications administered, and other factors known to those of skill in the art. An effective amount to treat the disease would broadly range (e.g., between about 0.001 mg and about 2000 mg per kg body weight of the recipient per day), and may be administered as a single dose or divided doses.

It is to be further understood that for any particular subject, specific dosage regimens should be adjusted over time according to the individual need and the professional judgment of the person administering or supervising the administration of the compositions.

The compositions are intended to be administered by a suitable route, including by way of example and without limitation, systemically (e.g., intravenously, intramuscularly, orally, intranasally, by inhalation, sublingually, mucosally, etc.), as described herein.

The disclosure further provides nucleic acids encoding said MABs and/or B virus epitopes. These nucleic acids may be present in a vector, which may further be present in a host cell (e.g., a bacterial cell, a yeast cell, or a eukaryotic cell). The nucleic acids may be codon-optimized to be efficiently expressed in the host cell, and/or may be expressed from a constitutive or inducible promoter.

The disclosure also provides humanized, recombinant and/or chimeric proteins comprising the B virus epitopes as described in Table 7. Said humanized, recombinant and/or chimeric proteins may be used in any of the compositions discussed herein. Said humanized, recombinant and/or chimeric protein may comprise one or more of the B virus epitopes as described in Table 7. The disclosure also provides vectors comprising nucleic acids encoding said humanized, recombinant and/or chimeric proteins.

The disclosure further contemplates that other MABs may be generated that specifically recognize the B virus epitopes described in Table 7 herein. These MABs may be included in any of the embodiments of the disclosure as described herein. These MABs may be humanized.

The disclosure also provides pharmaceutical compositions comprising the MABs and/or B virus epitopes, alone or in combination with a carrier and/or another therapeutic agent. In a further embodiment, the MABs and/or B virus epitopes are present in a therapeutically effective amount. In another embodiment, the pharmaceutical composition comprises (i) a vector comprising a nucleic acid encoding a MAB or a B virus epitope, (ii) a host cell comprising the MAB and/or B virus epitope, and/or (iii) a recombinant and/or chimeric protein comprising at least one B virus epitope. In some embodiments, the pharmaceutical composition may be a vaccine, and can comprise adjuvants and other pharmaceutically acceptable components.

The disclosure further provides methods of treating various simplexvirus infections using the compositions as described herein. Specifically, Rh-BV and/or Jap-BV infections may be treated with one or more of the compositions, preferably compositions comprising 12F5.C1 and/or 12G9.G5, and/or other MABs specific to the B virus epitopes recognized by 12F5.C1 and/or 12G9.G5. Further, Rh-BV, Jap-BV, PT-BV, and/or Cyno-BV infections may be treated with one or more of the compositions, preferably compositions comprising 7H1.G5, 18D10.F2.A4, 2G12.D12.D4, 6E10.D7, 5D10.C9, 5E10.C10, 7G9.E3, 10F9.F1, 7F7.G7, 5A2, 4E11, 9F1, 3H6, 7G2, 7G8, and/or 1G3, and/or other MABs specific to the B virus epitopes recognized by these MABs.

The method of treatment may comprise testing the subject to determine if the subject has a simplexvirus infection, determining the type of simplexvirus infection by using the MABs as described herein, and selecting an appropriate therapeutic composition and/or method.

The method of treatment may also comprise neutralizing the B virus by administration of a composition comprising 5A2 and/or 4E11 MABs or other MABs specific to the B virus epitopes recognized by these MABs.

In one embodiment of any of the compositions of the disclosure, the pharmaceutical composition is formulated for delivery by, for example and not limitation, oral, topical, rectal, mucosal, sublingual, nasal, parenteral routes and via naso/oro-gastric gavage, as well as parenteral, intraperitoneal, intradermal, transdermal, intrathecal, nasal, and intracheal administration. In one embodiment of any of the compositions of the disclosure, the composition is in a form of a liquid, foam, cream, spray, powder, or gel. In one embodiment of any of the compositions of the disclosure, the composition comprises a buffering agent (e.g., sodium bicarbonate, infant formula or sterilized human milk).

It is contemplated that when used to treat various diseases, the compositions and methods of the disclosure can be combined with other therapeutic agents suitable for the same or similar diseases. Also, two or more embodiments of the disclosure may be also co-administered to generate additive or synergistic effects. When co-administered with a second therapeutic agent, the embodiment of the disclosure and the second therapeutic agent may be simultaneously or sequentially (in any order). Suitable therapeutically effective dosages for each agent may be lowered due to the additive action or synergy.

The compositions and methods of the disclosure can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 41BB, OX40, etc.). The inhibitory treatments of the disclosure can be also combined with other treatments that possess the ability to modulate NKT function or stability, including but not limited to CD1d, CD1d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e), in any of the aforementioned forms or formulations, alone or in combination with each other or other agents. The inhibitory treatments of the disclosure can also be combined with other anti-viral therapeutic compositions and/or treatments, such as for example but not limitation, gancyclovir and/or acyclovir, and derivatives, modifications, analogs, and prodrugs thereof.

Therapeutic methods of the disclosure can be combined with additional immunotherapies and therapies.

The compositions of the disclosure can comprise a carrier and/or excipient. While it is possible to use a compound of the disclosure for therapy as is, it may be preferable to administer it in a pharmaceutical formulation, e.g., in admixture with a suitable pharmaceutical excipient and/or carrier selected with regard to the intended route of administration and standard pharmaceutical practice. The excipient and/or carrier must be “acceptable” in the sense of being compatible with the other ingredients of the formulation and not deleterious to the recipient thereof. Acceptable excipients and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient and carrier can be selected with regard to the intended route of administration and standard pharmaceutical practice. Oral formulations readily accommodate additional mixtures, such as, e.g., milk, yogurt, and infant formula. Solid dosage forms for oral administration can also be used and can include, e.g., capsules, tablets, caplets, pills, troches, lozenges, powders, and granules. Non-limiting examples of suitable excipients include, e.g., diluents, buffering agents (e.g., sodium bicarbonate, infant formula, sterilized human milk, or other agents which allow bacteria to survive and grow [e.g., survive in the acidic environment of the stomach and to grow in the intestinal environment]), preservatives, stabilizers, binders, compaction agents, lubricants, dispersion enhancers, disintegration agents, antioxidants, flavoring agents, sweeteners, and coloring agents. Those of relevant skill in the art are well able to prepare suitable solutions.

Administration of the compounds and compositions in the methods of the disclosure can be accomplished by any method known in the art. Non-limiting examples of useful routes of delivery include oral, rectal, fecal (by enema), and via naso/oro-gastric gavage, as well as parenteral, intraperitoneal, intradermal, transdermal, intrathecal, nasal, and intracheal administration. The active agent may be systemic after administration or may be localized by the use of regional administration, intramural administration, or use of an implant that acts to retain the active dose at the site of implantation. Bacteria can be mixed with a carrier and (for easier delivery to the digestive tract) applied to liquid or solid food, or feed or to drinking water. The carrier material should be non-toxic to the bacteria and the subject/patient. Non-limiting examples of bacteria-containing formulations useful in the methods of the disclosure include oral capsules and saline suspensions for use in feeding tubes, transmission via nasogastric tube, or enema. If live bacteria are used, the carrier should preferably contain an ingredient that promotes viability of the bacteria during storage. The formulation can include added ingredients to improve palatability, improve shelf-life, impart nutritional benefits, and the like. If a reproducible and measured dose is desired, the bacteria can be administered by a rumen cannula. In certain embodiments, the bacteria-containing formulation used in the methods of the disclosure further comprises a buffering agent. Examples of useful buffering agents include saline, sodium bicarbonate, milk, yogurt, infant formula, and other dairy products.

The useful dosages of the compounds and formulations of the disclosure will vary widely, depending upon the nature of the disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level.

For oral administration, the active ingredient(s) can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and nonaqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives.

Solutions or suspensions can include any of the following components, in any combination: a sterile diluent, including by way of example without limitation, water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity, such as sodium chloride or dextrose.

In instances in which the agents exhibit insufficient solubility, methods for solubilizing agents may be used. Such methods are known to those of skill in this art, and include, but are not limited to, using co-solvents, such as, e.g., dimethylsulfoxide (DMSO), using surfactants, such as TWEEN® 80, or dissolution in aqueous sodium bicarbonate. Pharmaceutically acceptable derivatives of the agents may also be used in formulating effective pharmaceutical compositions.

The composition can contain along with the active agent, for example and without limitation: a diluent such as lactose, sucrose, dicalcium phosphate, or carboxymethylcellulose; a lubricant, such as magnesium stearate, calcium stearate and talc; and a binder such as starch, natural gums, such as gum acacia gelatin, glucose, molasses, polyvinylpyrrolidone, celluloses and derivatives thereof, povidone, crospovidones and other such binders known to those of skill in the art. Liquid pharmaceutically administrable compositions can, for example, be prepared by dissolving, dispersing, or otherwise mixing an active agent as defined above and optional pharmaceutical adjuvants in a carrier, such as, by way of example and without limitation, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, such as, by way of example and without limitation, acetate, sodium citrate, cyclodextrin derivatives, sorbitan monolaurate, triethanolamine sodium acetate, triethanolamine oleate, and other such agents. Actual methods of preparing such dosage forms are known, or will be apparent, to those skilled in this art (e.g., Remington's Pharmaceutical Sciences, Mack Publishing Company, Easton, Pa., 15th Edition, 1975). The composition or formulation to be administered will, in any event, contain a quantity of the active agent in an amount sufficient to alleviate the symptoms of the treated subject.

The active agents or pharmaceutically acceptable derivatives may be prepared with carriers that protect the agent against rapid elimination from the body, such as time release formulations or coatings. The compositions may include other active agents to obtain desired combinations of properties.

Oral pharmaceutical dosage forms include, by way of example and without limitation, solid, gel and liquid. Solid dosage forms include tablets, capsules, granules, and bulk powders. Oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, such as capsules or tablets. The tablets, pills, capsules, troches and the like can contain any of the following ingredients, or agents of a similar nature: a binder; a diluent; a disintegrating agent; a lubricant; a glidant; a sweetening agent; and a flavoring agent.

Examples of binders include, by way of example and without limitation, microcrystalline cellulose, gum tragacanth, glucose solution, acacia mucilage, gelatin solution, sucrose, and starch paste. Lubricants include, by way of example and without limitation, talc, starch, magnesium or calcium stearate, lycopodium and stearic acid. Diluents include, by way of example and without limitation, lactose, sucrose, starch, kaolin, salt, mannitol, and dicalcium phosphate. Glidants include, by way of example and without limitation, colloidal silicon dioxide. Disintegrating agents include, by way of example and without limitation, crosscarmellose sodium, sodium starch glycolate, alginic acid, corn starch, potato starch, bentonite, methylcellulose, agar and carboxymethylcellulose. Coloring agents include, by way of example and without limitation, any of the approved certified water soluble FD and C dyes, mixtures thereof; and water insoluble FD and C dyes suspended on alumina hydrate. Sweetening agents include, by way of example and without limitation, sucrose, lactose, mannitol and artificial sweetening agents such as saccharin, and any number of spray dried flavors. Flavoring agents include, by way of example and without limitation, natural flavors extracted from plants such as fruits and synthetic blends of agents which produce a pleasant sensation, such as, but not limited to peppermint and methyl salicylate. Wetting agents include, by way of example and without limitation, propylene glycol monostearate, sorbitan monooleate, diethylene glycol monolaurate, and polyoxyethylene laural ether. Emetic-coatings include, by way of example and without limitation, fatty acids, fats, waxes, shellac, ammoniated shellac and cellulose acetate phthalates. Film coatings include, by way of example and without limitation, hydroxyethylcellulose, sodium carboxymethylcellulose, polyethylene glycol 4000 and cellulose acetate phthalate.

If oral administration is desired, the agent could be provided in a composition that protects it from the acidic environment of the stomach. For example, the composition can be formulated in an enteric coating that maintains its integrity in the stomach and releases the active agent in the intestine. The composition may also be formulated in combination with an antacid or other such ingredient.

When the dosage unit form is a capsule, it can contain, in addition to material of the above type, a liquid carrier such as fatty oil. In addition, dosage unit forms can contain various other materials which modify the physical form of the dosage unit, for example, coatings of sugar and other enteric agents. The agents can also be administered as a component of an elixir, suspension, syrup, wafer, sprinkle, chewing gum or the like. A syrup may contain, in addition to the active agents, sucrose as a sweetening agent and certain preservatives, dyes and colorings and flavors. The active materials can also be mixed with other active materials which do not impair the desired action, or with materials that supplement the desired action, such as antacids, H2 blockers, and diuretics.

Pharmaceutically acceptable carriers included in tablets are binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar-coated, multiple compressed and chewable tablets. Flavoring and sweetening agents are useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents may be used in any of the above dosage forms.

Solvents include, by way of example and without limitation, glycerin, sorbitol, ethyl alcohol and syrup. Examples of preservatives include, without limitation, glycerin, methyl and propylparaben, benzoic acid, sodium benzoate and alcohol. Non-aqueous liquids utilized in emulsions include, by way of example and without limitation, mineral oil and cottonseed oil. Emulsifying agents include, by way of example and without limitation, gelatin, acacia, tragacanth, bentonite, and surfactants such as polyoxyethylene sorbitan monooleate. Suspending agents include, by way of example and without limitation, sodium carboxymethylcellulose, pectin, tragacanth, Veegum and acacia. Diluents include, by way of example and without limitation, lactose and sucrose. Sweetening agents include, by way of example and without limitation, sucrose, syrups, glycerin and artificial sweetening agents such as saccharin. Wetting agents include, by way of example and without limitation, propylene glycol monostearate, sorbitan monooleate, diethylene glycol monolaurate, and polyoxyethylene lauryl ether. Organic acids include, by way of example and without limitation, citric and tartaric acid. Sources of carbon dioxide include, by way of example and without limitation, sodium bicarbonate and sodium carbonate. Coloring agents include, by way of example and without limitation, any of the approved certified water soluble FD and C dyes, and mixtures thereof. Flavoring agents include, by way of example and without limitation, natural flavors extracted from plants, such as fruits, and synthetic blends of agents which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos. 4,328,245, 4,409,239, and 4,410,545. For a liquid dosage form, the solution (e.g., in a polyethylene glycol) may be diluted with a sufficient quantity of a pharmaceutically acceptable liquid carrier (e.g., water) to be easily measured for administration.

Alternatively, liquid or semi-solid oral formulations may be prepared by dissolving or dispersing the active agent or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. RE28,819 and U.S. Pat. No. 4,358,603. Briefly, such formulations include, but are not limited to, those containing an agent provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes, such as acetaldehyde diethyl acetal.

Tablets and capsules formulations may be coated as known by those of skill in the art in order to modify or sustain dissolution of the active ingredient. Thus, for example and without limitation, they may be coated with a conventional enterically digestible coating, such as phenylsalicylate, waxes and cellulose acetate phthalate.

Parenteral administration, generally characterized by injection, either subcutaneously, intramuscularly or intravenously, is also contemplated herein. Injectables can be prepared in conventional forms, either as liquid solutions or suspensions, solid forms suitable for solution or suspension in liquid prior to injection, or as emulsions. Suitable excipients include, by way of example and without limitation, water, saline, dextrose, glycerol or ethanol. In addition, if desired, the pharmaceutical compositions to be administered may also contain minor amounts of non-toxic auxiliary substances, such as wetting or emulsifying agents, pH buffering agents, stabilizers, solubility enhancers, and other such agents, such as, for example, sodium acetate, sorbitan monolaurate, triethanolamine oleate and cyclodextrins.

Implantation of a slow-release or sustained-release system, such that a constant level of dosage is maintained (e.g., U.S. Pat. No. 3,710,795) is also contemplated herein. Briefly, an inhibitor of Nt5e or A1R is dispersed in a solid inner matrix (e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate) that is surrounded by an outer polymeric membrane (e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer) that is insoluble in body fluids. The agent diffuses through the outer polymeric membrane in a release rate controlling step. The percentage of active agent contained in such parenteral compositions is highly dependent on the specific nature thereof, as well as the activity of the agent and the needs of the subject.

Lyophilized powders can be reconstituted for administration as solutions, emulsions, and other mixtures or formulated as solids or gels. The sterile, lyophilized powder is prepared by dissolving an agent provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial will contain, by way of example and without limitation, a single dosage (10-1000 mg, such as 100- 500 mg) or multiple dosages of the agent. The lyophilized powder can be stored under appropriate conditions, such as at about 4° C. to room temperature. Reconstitution of this lyophilized powder with water for injection provides a formulation for use in parenteral administration. For reconstitution, about 1-50 mg, such as about 5-35 mg, for example, about 9-30 mg of lyophilized powder, is added per mL of sterile water or other suitable carrier. The precise amount depends upon the selected agent. Such amount can be empirically determined.

The inventive composition or pharmaceutically acceptable derivatives thereof may be formulated as aerosols for application e.g., by inhalation or intranasally (e.g., as described in U.S. Pat. Nos. 4,044,126, 4,414,209, and 4,364,923). These formulations can be in the form of an aerosol or solution for a nebulizer, or as a microfine powder for insufflation, alone or in combination with an inert carrier such as lactose. In such a case, the particles of the formulation will, by way of example and without limitation, have diameters of less than about 50 microns, such as less than about 10 microns.

The agents may be also formulated for local or topical application, such as for application to the skin and mucous membranes (e.g., intranasally), in the form of nasal solutions, gels, creams, and lotions.

Other routes of administration, such as transdermal patches are also contemplated herein. Transdermal patches, including iontophoretic and electrophoretic devices, are well known to those of skill in the art. For example, such patches are disclosed in U.S. Pat. Nos. 6,267,983, 6,261,595, 6,256,533, 6,167,301, 6,024,975, 6,010,715, 5,985,317, 5,983,134, 5,948,433, and 5,860,957.

### Diagnostic and Therapeutic Methods of the Disclosure

It is contemplated that the B virus epitopes and monoclonal antibodies (MABs) described herein can be used to diagnose whether a subject is infected with a simplexvirus, preferably a B virus. It is further contemplated that said B virus epitopes and MABs may be specific to certain viral subtypes/strains, enabling differential diagnosis of specific simplexvirus types, including specific B virus strains. The disclosure further provides that a suitable treatment composition or method may be selected based on the results of the diagnostic assay.

The disclosure provides that the MABs described in Table 1, as well as any other MABs recognizing the B virus epitopes described in Table 7, may be used to diagnose whether a subject is infected with a simplexvirus, preferably a B virus. The method comprises taking a bodily fluid sample (e.g., blood, serum, plasma, urine, saliva, and/or CSF) from a subject, determining whether the sample contains a simplexvirus, and determining whether the subject is infected with a simplexvirus.

In one embodiment, the disclosure provides for a differential diagnosis among several simplexviruses that can infect a subject, such as for example and not limitation, Rh-BV, Cyno-BV, PT-BV, Jap-BV, HSV-1, and/or HSV-2.

In a specific embodiment, the diagnostic method allows differential diagnosis of Rh-BV and Jap-BV strains by using MABs 12F5.C1 and/or 12G9.G5 as described in Table 1, and/or other MABs recognizing the B virus epitopes of these MABs as described in Table 7.

In another specific embodiment, the diagnostic method allows specific diagnosis of one or more of Rh-BV, Cyno-BV, PT-BV, and/or Jap-BV by using one or more of MABs 1G3, 1G3.C1, 9F1, 3H6, and/or 7G2, and/or other MABs recognizing the B virus epitopes (e.g., the gB region of B virus) of these MABs as described in Table 7. This method prevents cross-reactivity with any other simplexvirus including HSV-1 and/or HSV-2, and thus allows for differential diagnosis of these B virus strains in a human subject who may also be infected with HSV-1 and/or HSV-2. Preferably, this embodiment of the method is performed on a sample obtained from a human subject.

In another specific embodiment, the diagnostic method allows specific diagnosis of one or more of Rh-BV, Cyno-BV, PT-BV, and/or Jap-BV by using one or more of MABs 2G12.D12.D4 and/or 6E10.D7, and/or other MABs recognizing the B virus epitopes (e.g., the gD region of B virus) of these MABs as described in Table 7. This method prevents cross-reactivity with any other simplexvirus including HSV-1 and/or HSV-2, and thus allows for differential diagnosis of these B virus strains in a human subject who may also be infected with HSV-1 and/or HSV-2. Preferably, this embodiment of the method is performed on a sample obtained from a human subject.

In another specific embodiment, the diagnostic method allows specific diagnosis of one or more of Rh-BV, Cyno-BV, PT-BV, and/or Jap-BV by using one or more of MABs 5E10.C10, 7G8, and/or 7F7.G7, and/or other MABs recognizing the B virus epitopes (e.g., BV-gB) of these MABs as described in Table 7. This method prevents cross-reactivity with any other simplexvirus including HSV-1 and/or HSV-2, and thus allows for differential diagnosis of these B virus strains in a human subject who may also be infected with HSV-1 and/or HSV-2. Preferably, this embodiment of the method is performed on a sample obtained from a human subject. In a further embodiment of this method, comparison of results from 5E10.C10 and/or 7G8 assays with the 7F7.G7 assay enable diagnosis of infection with the PT-BV strain, as 7F7.G7 does not react to this specific strain.

In yet another specific embodiment, the diagnostic method allows differential diagnosis of one or more of Rh-BV, Cyno-BV, PT-BV, Jap-BV, HSV-1 and/or HSV-2 relative to other simian simplexviruses and human simplexviruses by using one or more of MABs 18.D10.F2.A4, 7H1.G5, 5D10.C9, 7G9.E3, 5A2, and/or 4E11, and/or other MABs recognizing the B virus epitopes (e.g., BV-gB and HSV1-gB) of these MABs as described in Table 7. This method prevents cross-reactivity with any other simian or human simplexvirus.

In any of the above embodiments of the disclosure, the step of determining whether the sample contains simplexviruses involves a hybridization assay (e.g., ELISA and/or Western blotting, including the variations of each described herein).

In any of the above embodiments of the disclosure, the method may further comprise selecting a therapeutic composition and/or method based on the results of the diagnostic assay.

In any of the above embodiments of the disclosure, the method may further comprise using least one of the antibody variable region amino acid sequences shown in SEQ ID NOs: 1-6 (and/or encoded by the nucleic acid sequences shown in SEQ ID NOs: 7-12, or individual CDRs thereof or related CDR sequences, as specified in more detail below.

In any of the above embodiments of the disclosure, the method may further comprise using least one of the B virus epitope sequences shown in SEQ ID NOs: 13-85 (and/or homologous variants thereof, such as amino acid sequences having at least 95%, 96%, 97%, 98%, 99%, or 100% identical to any of SEQ ID NOs: 13-85).

In any of the above embodiments of the disclosure, the method may further comprise using anti-B virus antibodies and fragments thereof that bind specifically and/or differentially to B viruses and comprise a light chain variable region having an amino acid sequence that is at least 95%, 96%, 97%, 98%, 99%, or 100% identical to any of SEQ ID NOs: 1, 3 and 5, or is a B virus-binding fragment or a homologous variant thereof, of an antibody comprised in said sequences. Alternatively, the anti-B virus antibodies and fragments thereof may be encoded by a nucleic acid having a nucleic acid sequence that is at least 95%, 96%, 97%, 98%, 99%, or 100% identical to any of SEQ ID NOs: 7, 9 and 11.

In any of the above embodiments of the disclosure, the method may further comprise using anti-B virus antibodies and fragments thereof that bind specifically and/or differentially to B viruses and comprise a heavy chain variable region having an amino acid sequence that is at least 95%, 96%, 97%, 98%, 99%, or 100% identical to any of SEQ ID NOs: 2, 4, and 6, or is a B virus-binding fragment or a homologous variant thereof, of an antibody comprised in said sequences. Alternatively, the anti-B virus antibodies and fragments thereof may be encoded by a nucleic acid having a nucleic acid sequence that is at least 95%, 96%, 97%, 98%, 99%, or 100% identical to any of SEQ ID NOs: 8, 10 and 12.

In any of the above embodiments of the disclosure, the method may further comprise using an antibody or an antibody fragment that binds specifically and/or differentially to a B virus or a fragment thereof, wherein the antibody or fragment thereof comprises a variable domain comprising a heavy chain (VH) comprising the amino acid sequence of SEQ ID NO: 2, 4, or 6 and a light chain (VL) comprising the amino acid sequence of SEQ ID NO: 1, 3, or 5. Alternatively, the VH region may be encoded by a nucleic acid comprising the nucleic acid sequence of SEQ ID NO: 8, 10, or 12 and a light chain (VL) comprising the amino acid sequence of SEQ ID NO: 7, 9, or 11.

In any of the above embodiments of the disclosure, the method may further comprise using an antibody or an antibody fragment that binds specifically and/or differentially to a B virus or a fragment thereof, wherein the antibody or fragment thereof comprises (a) a VH sequence having at least 95%, 96%, 97%, 98%, 99%, or 100% sequence identity to the amino acid sequence of SEQ ID NO: 2, 4, or 6; or (b) a VL sequence having at least 9595%, 96%, 97%, 98%, 99%, or 100% sequence identity to the amino acid sequence of SEQ ID NO: 1, 3, or 5.

In any of the above embodiments of the disclosure, the method may further comprise using an antibody or an antibody fragment that binds specifically and/or differentially to a B virus or a fragment thereof, wherein the antibody or fragment thereof comprises (a) a VH sequence encoded by a nucleic acid having at least 95%, 96%, 97%, 98%, 99%, or 100% sequence identity to the nucleic acid sequence of SEQ ID NO: 8, 10, or 12; or (b) a VL sequence having at least 95%, 96%, 97%, 98%, 99%, or 100% sequence identity to the amino acid sequence of SEQ ID NO: 7, 9, or 11.

In any of the above embodiments of the disclosure, the method may further comprise using an antibody or an antibody fragment that binds specifically and/or differentially to a B virus or a fragment thereof, wherein the antibody comprises a VH comprising the amino acid sequence of SEQ ID NO: 2 (or the VH region may be encoded by a nucleic acid comprising the nucleic acid sequence of SEQ ID NO: 8). In certain embodiments, the antibody or fragment thereof further comprises a VL comprising the amino acid sequence of SEQ ID NO: 1 (or the VL region may be encoded by a nucleic acid comprising the nucleic acid sequence of SEQ ID NO: 7). In certain embodiments, the antibody or fragment thereof comprises a VH comprising the amino acid sequence of SEQ ID NO: 2 and a VL comprising the amino acid sequence of SEQ ID NO: 1. In certain embodiments, the antibody or fragment thereof comprises a VH encoded by a nucleic acid comprising the nucleic acid sequence of SEQ ID NO: 8 and a VL region encoded by a nucleic acid comprising the nucleic acid sequence of SEQ ID NO: 7.

In any of the above embodiments of the disclosure, the method may further comprise using an antibody or an antibody fragment that binds specifically and/or differentially to a B virus or a fragment thereof, wherein the antibody comprises a VH comprising the amino acid sequence of SEQ ID NO: 4 (or the VH region may be encoded by a nucleic acid comprising the nucleic acid sequence of SEQ ID NO: 10). In certain embodiments, the antibody or fragment thereof further comprises a VL comprising the amino acid sequence of SEQ ID NO: 3 (or the VL region may be encoded by a nucleic acid comprising the nucleic acid sequence of SEQ ID NO: 9). In certain embodiments, the antibody or fragment thereof comprises a VH comprising the amino acid sequence of SEQ ID NO: 4 and a VL comprising the amino acid sequence of SEQ ID NO: 3. In certain embodiments, the antibody or fragment thereof comprises a VH encoded by a nucleic acid comprising the nucleic acid sequence of SEQ ID NO: 10 and a VL region encoded by a nucleic acid comprising the nucleic acid sequence of SEQ ID NO: 9.

In any of the above embodiments of the disclosure, the method may further comprise using an antibody or an antibody fragment that binds specifically and/or differentially to a B virus or a fragment thereof, wherein the antibody comprises a VH comprising the amino acid sequence of SEQ ID NO: 6 (or the VH region may be encoded by a nucleic acid comprising the nucleic acid sequence of SEQ ID NO: 12). In certain embodiments, the antibody or fragment thereof further comprises a VL comprising the amino acid sequence of SEQ ID NO: 5 (or the VL region may be encoded by a nucleic acid comprising the nucleic acid sequence of SEQ ID NO: 11). In certain embodiments, the antibody or fragment thereof comprises a VH comprising the amino acid sequence of SEQ ID NO: 6 and a VL comprising the amino acid sequence of SEQ ID NO: 5. In certain embodiments, the antibody or fragment thereof comprises a VH encoded by a nucleic acid comprising the nucleic acid sequence of SEQ ID NO: 12 and a VL region encoded by a nucleic acid comprising the nucleic acid sequence of SEQ ID NO: 11.

In any of the above embodiments of the disclosure, the method may further comprise using an antibody or an antibody fragment that binds specifically and/or differentially to a B virus or a fragment thereof, wherein the antibody comprises a VH comprising an amino acid sequence having at least 95%, 96%, 97%, 98%, 99%, or 100% sequence identity to the amino acid sequence of SEQ ID NO: 2 (or the VH region may be encoded by a nucleic acid comprising a nucleic acid sequence having at least 95%, 96%, 97%, 98%, 99%, or 100% sequence identity to the nucleic acid sequence of SEQ ID NO: 8). In certain embodiments, the antibody or fragment thereof further comprises a VL comprising an amino acid sequence having at least 95%, 96%, 97%, 98%, 99%, or 100% sequence identity to the amino acid sequence of SEQ ID NO: 1 (or the VL region may be encoded by a nucleic acid comprising a nucleic acid sequence having at least 95%, 96%, 97%, 98%, 99%, or 100% sequence identity to the nucleic acid sequence of SEQ ID NO: 7). In certain embodiments, the antibody or fragment thereof comprises a VH comprising an amino acid sequence having at least 95%, 96%, 97%, 98%, 99%, or 100% sequence identity to the amino acid sequence of SEQ ID NO: 2 and a VL comprising an amino acid sequence having at least 95%, 96%, 97%, 98%, 99%, or 100% sequence identity to the amino acid sequence of SEQ ID NO: 1. In certain embodiments, the antibody or fragment thereof comprises a VH encoded by a nucleic acid comprising a nucleic acid sequence having at least 95%, 96%, 97%, 98%, 99%, or 100% sequence identity to the nucleic acid sequence of SEQ ID NO: 8 and a VL region encoded by a nucleic acid comprising a nucleic acid sequence having at least 95%, 96%, 97%, 98%, 99%, or 100% sequence identity to the nucleic acid sequence of SEQ ID NO: 7.

In any of the above embodiments of the disclosure, the method may further comprise using an antibody or an antibody fragment that binds specifically and/or differentially to a B virus or a fragment thereof, wherein the antibody comprises a VH comprising an amino acid sequence having at least 95%, 96%, 97%, 98%, 99%, or 100% sequence identity to the amino acid sequence of SEQ ID NO: 4 (or the VH region may be encoded by a nucleic acid comprising a nucleic acid sequence having at least 95%, 96%, 97%, 98%, 99%, or 100% sequence identity to the nucleic acid sequence of SEQ ID NO: 10). In certain embodiments, the antibody or fragment thereof further comprises a VL comprising an amino acid sequence having at least 95%, 96%, 97%, 98%, 99%, or 100% sequence identity to the amino acid sequence of SEQ ID NO: 3 (or the VL region may be encoded by a nucleic acid comprising a nucleic acid sequence having at least 95%, 96%, 97%, 98%, 99%, or 100% sequence identity to the nucleic acid sequence of SEQ ID NO: 9). In certain embodiments, the antibody or fragment thereof comprises a VH comprising an amino acid sequence having at least 95%, 96%, 97%, 98%, 99%, or 100% sequence identity to the amino acid sequence of SEQ ID NO: 4 and a VL comprising an amino acid sequence having at least 95%, 96%, 97%, 98%, 99%, or 100% sequence identity to the amino acid sequence of SEQ ID NO: 3. In certain embodiments, the antibody or fragment thereof comprises a VH encoded by a nucleic acid comprising a nucleic acid sequence having at least 95%, 96%, 97%, 98%, 99%, or 100% sequence identity to the nucleic acid sequence of SEQ ID NO: 10 and a VL region encoded by a nucleic acid comprising a nucleic acid sequence having at least 95%, 96%, 97%, 98%, 99%, or 100% sequence identity to the nucleic acid sequence of SEQ ID NO: 9.

In any of the above embodiments of the disclosure, the method may further comprise using an antibody or an antibody fragment that binds specifically and/or differentially to a B virus or a fragment thereof, wherein the antibody comprises a VH comprising an amino acid sequence having at least 95%, 96%, 97%, 98%, 99%, or 100% sequence identity to the amino acid sequence of SEQ ID NO: 6 (or the VH region may be encoded by a nucleic acid comprising a nucleic acid sequence having at least 95%, 96%, 97%, 98%, 99%, or 100% sequence identity to the nucleic acid sequence of SEQ ID NO: 12). In certain embodiments, the antibody or fragment thereof further comprises a VL comprising an amino acid sequence having at least 95%, 96%, 97%, 98%, 99%, or 100% sequence identity to the amino acid sequence of SEQ ID NO: 5 (or the VL region may be encoded by a nucleic acid comprising a nucleic acid sequence having at least 95%, 96%, 97%, 98%, 99%, or 100% sequence identity to the nucleic acid sequence of SEQ ID NO: 11). In certain embodiments, the antibody or fragment thereof comprises a VH comprising an amino acid sequence having at least 95%, 96%, 97%, 98%, 99%, or 100% sequence identity to the amino acid sequence of SEQ ID NO: 6 and a VL comprising an amino acid sequence having at least 95%, 96%, 97%, 98%, 99%, or 100% sequence identity to the amino acid sequence of SEQ ID NO: 5. In certain embodiments, the antibody or fragment thereof comprises a VH encoded by a nucleic acid comprising a nucleic acid sequence having at least 95%, 96%, 97%, 98%, 99%, or 100% sequence identity to the nucleic acid sequence of SEQ ID NO: 12 and a VL region encoded by a nucleic acid comprising a nucleic acid sequence having at least 95%, 96%, 97%, 98%, 99%, or 100% sequence identity to the nucleic acid sequence of SEQ ID NO: 11.

In another aspect, the disclosure provides a method of treatment of a simplexvirus infection in a subject in need thereof, comprising administering any composition of the foregoing embodiments.

In some embodiments, the method further comprises a diagnostic assay to determine whether the subject has a simplexvirus infection using the antibodies. In other embodiments, the diagnostic assay can provide a differential diagnosis of the simplexvirus infection from the group comprising Rh-BV, Cyno-BV, PT-BV, Jap-BV, HSV-1 and/or HSV-2.

In some embodiments, the pharmaceutical composition is a vaccine. In other embodiments, the method of treatment comprises administering a vaccine.

In some embodiments, the method comprises treatment of Rh-BV, Jap-BV, PT-BV, and/or Cyno-BV infections with compositions according to any of claims 36-95 comprising:

one or more of anti-B virus antibody and/or fragment thereof as described herein;

B virus epitopes recognized by said MABs;

and/or other MABs specific to the B virus epitopes recognized by said MABs.

In other embodiments, the method comprises neutralizing the B virus by administration of a composition according to any of claims 36-95 comprising:

one or more of anti-B virus antibody and/or fragment thereof as described herein;

B virus epitopes recognized by said MABs; and/or

other MABs specific to the B virus epitopes recognized by said MABs.

In some embodiments, the disclosure provides a method of diagnosing a subject with a simplexvirus infection comprising use of one or more of anti-B virus antibody and/or fragment thereof as described herein. In certain embodiments, the simplexvirus infection is a B virus infection, a HSV-1 infection, and/or a HSV-2 infection.

In some embodiments, the method further comprises the steps of:

(i) taking a bodily fluid sample (e.g., blood, serum, plasma, urine, saliva, and/or CSF) from a subject;

(ii) determining whether the sample contains a simplexvirus; and

(iii) determining whether the subject is infected with a simplexvirus.

In other embodiments, the method is used to make a differential diagnosis among Rh-BV, Cyno-BV, PT-BV, Jap-BV, HSV-1, and/or HSV-2.

In certain embodiments, the step of determining whether the sample contains simplexviruses involves a hybridization assay.

In other embodiments, the method further comprises selecting a therapeutic composition and/or method based on the results of step (iii).

Any embodiment described herein or any combination thereof applies to any and all anti-B antibodies or fragments thereof, methods and uses of the disclosure described herein.

In any of the foregoing methods, the method further comprises treatment and/or prophylaxis of subjects determined to have, or be at risk for having, a B virus.

In one embodiment of any of the foregoing methods, the treatment is prophylactic and comprises administration of appropriate prophylactically effective pharmaceutical compositions and/or use of appropriate prophylactically effective methods. In some embodiments, the prophylactic treatment comprises at least one anti-B virus antibody or fragment thereof or B virus epitope as described herein. In still other embodiments, the prophylactic treatment comprises a therapeutic agent and at least one anti-B virus antibody or fragment thereof or B virus epitope as described herein.

In one embodiment of any of the foregoing methods, the treatment comprises administration of appropriate therapeutically effective pharmaceutical compositions and/or use of appropriate therapeutically effective methods. In some embodiments, the therapeutic treatment comprises at least one anti-B virus antibody or fragment thereof or B virus epitope as described herein. In still other embodiments, the therapeutic treatment comprises a therapeutic agent and at least one anti-B virus antibody or fragment thereof or B virus epitope as described herein.

The treatment and/or prophylaxis of a subject having or at risk for having a condition and/or disease associated with a B virus may be administered to the subject via any suitable route of administration. The effective amount or dose of such treatment and/or prophylaxis administered should be sufficient to provide a therapeutic or prophylactic response in the subject over a reasonable time frame. For example, the dose of immunosuppressive drug should be sufficient to decrease symptoms of such condition and/or disease. The dose will be determined by the efficacy of the particular active agent and the condition of the subject (e.g., human), as well as the body weight of the subject (e.g., human) to be treated.

### Kits

In one aspect, the present disclosure provides a kit for diagnosing and/or detecting a condition or disease associated with a B virus in a subject, said kit comprising probes directed towards a B virus. The kit can also comprise (i) a detection means and/or (ii) an amplification means. The kit may further optionally include control probe sets.

In another aspect, the present disclosure provides a kit for diagnosing and/or detecting a condition or disease associated with a B virus in a subject, said kit comprising pairs of oligonucleotides directed towards the B virus, wherein the pairs of oligonucleotides may be used to determine the expression level of the B virus. The kit can also comprise (i) a detection means and/or (ii) an amplification means. The kit may further optionally include control primer/oligonucleotide sets for detection of control RNA in order to provide a control level as described herein.

In some embodiments, these kits comprise detection reagents that specifically bind a B virus and fragments thereof. The kits typically include a probe that comprises an antibody or nucleic acid sequence that specifically binds to polypeptides or polynucleotides of the disclosure, such as for example and not limitation, B viruses and fragments thereof as well as genes encoding this biomarker and a label for detecting the presence of the probe. The kits may include several antibodies specific for, or polynucleotide sequences encoding, the polypeptides of the disclosure. The kits may further comprise control probes for detection of a control nucleic acid or a control protein in order to provide a control level of the nucleic acid or protein, and/or other standards or controls. The probe is optionally detectably labeled.

The kit may contain in separate containers a nucleic acid or antibody (either already bound to a solid matrix or packaged separately with reagents for binding them to the matrix), control formulations (positive and/or negative), and/or a detectable label such as fluorescein, green fluorescent protein, rhodamine, cyanine dyes, Alexa dyes, luciferase, radiolabels, among others. Instructions for carrying out the assay may also be included in the kit. The assay may, for example and not limitation, be in the form of a Northern hybridization, sandwich ELISA or protein antibody array.

Reagents for detecting biomarkers of the present disclosure can be immobilized on a solid matrix such as a porous strip to form at least one biomarker detection site. The measurement or detection region of the porous strip may include a plurality of sites containing an antibody or nucleic acid. A test strip may also contain sites for negative and/or positive controls. Alternatively, control sites can be located on a separate strip from the test strip. Optionally, the different detection sites may contain different amounts of immobilized antibodies or nucleic acids, e.g., a higher amount in the first detection site and lesser amounts in subsequent sites. Upon the addition of test sample, the number of sites displaying a detectable signal provides a quantitative indication of the amount of biomarker present in the sample. The detection sites may be configured in any suitably detectable shape and are typically in the shape of a bar or dot spanning the width of a test strip.

Alternatively, the kit contains a nucleic acid substrate array comprising one or more nucleic acid sequences. The nucleic acids on the array specifically identify one or more nucleic acid sequences adapted to bind a nucleic acid sequence encoding part of a B virus or a homologous variant thereof The substrate array can be on, e.g., a solid substrate or “chip”. Alternatively, the substrate array can be a solution array.

A kit of the disclosure can also provide reagents for primer extension and amplification reactions. For example, in some embodiments, the kit may further include one or more of the following components: a reverse transcriptase enzyme, a DNA polymerase enzyme (such as, e.g., a thermostable DNA polymerase), a polymerase chain reaction buffer, a reverse transcription buffer, and deoxynucleoside triphosphates (dNTPs).

Alternatively (or in addition), a kit can include reagents for performing a hybridization assay for nucleic acid(s) and/or proteins. The detecting agents can include nucleotide analogs and/or a labeling moiety, e.g., directly detectable moiety such as a fluorophore (fluorochrome) or a radioactive isotope, or indirectly detectable moiety, such as a member of a binding pair, such as biotin, or an enzyme capable of catalyzing a non-soluble colorimetric or luminometric reaction. In addition, the kit may further include at least one container containing reagents for detection of electrophoresed nucleic acids. Such reagents include those which directly detect nucleic acids, such as fluorescent intercalating agent or silver staining reagents, or those reagents directed at detecting labeled nucleic acids, such as, but not limited to, ECL reagents. A kit can further include DNA or RNA isolation or purification means as well as positive and negative controls. Alternatively, the kit may include at least one container containing reagents for detection of electrophoresed proteins. Such reagents include those which directly detect proteins, such as Coomassie blue or other staining reagents including fluorescent staining agents, or those reagents directed at detecting labeled proteins. A kit can further include protein isolation or purification means as well as positive and negative controls. A kit can also include a notice associated therewith in a form prescribed by a governmental agency regulating the manufacture, use or sale of diagnostic kits. Detailed instructions for use, storage and trouble-shooting may also be provided with the kit. A kit can also be optionally provided in a suitable housing that is preferably useful for robotic handling in a high throughput setting.

The components of the kit may be provided as dried powder(s). When reagents and/or components are provided as a dry powder, the powder can be reconstituted by the addition of a suitable solvent. It is envisioned that the solvent may also be provided in another container. The container will generally include at least one vial, test tube, flask, bottle, syringe, and/or other container means, into which the solvent is placed, optionally aliquoted. The kits may also comprise a second container means for containing a sterile, pharmaceutically acceptable buffer and/or other solvent.

Where there is more than one component in the kit, the kit also will generally contain a second, third, or other additional container into which the additional components may be separately placed. However, various combinations of components may be comprised in a container.

Such kits may also include components that preserve or maintain DNA or RNA, such as reagents that protect against nucleic acid degradation. Such components may be nuclease or RNase-free or protect against RNases, for example. Such kits may also include components that preserve or maintain proteins, such as reagents that protect against protein degradation. Any of the compositions or reagents described herein may be components in a kit.

In some embodiments, the kit further comprises an apparatus for collecting a blood sample from a subject. In other embodiments, the kit further comprises instructions for using the collection apparatus and/or the reagents comprising the kit.

### Computer-Implemented Aspects

As understood by those of ordinary skill in the art, the methods and information described herein may be implemented, in all or in part, as computer executable instructions on known computer readable media. For example, the methods described herein may be implemented in hardware. Alternatively, the method may be implemented in software stored in, for example, one or more memories or other computer readable medium and implemented on one or more processors. As is known, the processors may be associated with one or more Controllers, calculation units and/or other units of a computer system, or implanted in firmware as desired. If implemented in software, the routines may be stored in any computer readable memory such as in RAM, ROM, flash memory, a magnetic disk, a laser disk, or other storage medium, as is also known. Likewise, this software may be delivered to a computing device via any known delivery method including, for example, over a communication channel such as a telephone line, the Internet, a wireless connection, etc., or via a transportable medium, such as a computer readable disk, flash drive, and the like.

More generally, and as understood by those of ordinary skill in the art, the various steps described above may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

When implemented in software, the software may be stored in any known computer readable medium such as on a magnetic disk, an optical disk, or other storage medium, in a RAM or ROM or flash memory of a computer, processor, hard disk drive, optical disk drive, tape drive, etc. Likewise, the software may be delivered to a user or a computing system via any known delivery method including, for example, on a computer readable disk or other transportable computer storage mechanism.

Thus, another aspect of the disclosure is a system that is capable of carrying out a part or all of a method of the disclosure, or carrying out a variation of a method of the disclosure as described herein in greater detail. Exemplary systems include, as one or more components, computing systems, environments, and/or configurations that may be suitable for use with the methods and include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like. In some variations, a system of the disclosure includes one or more machines used for analysis of biological material (e.g., genetic material), as described herein. In some variations, this analysis of the biological material involves a chemical analysis and/or a nucleic acid amplification.

The computer may operate in a networked environment using logical connections to one or more remote computers, such as a remote computer via a network interface controller (NIC). The remote computer may be a personal computer, a server, a router, a network PC, a peer device or other common network node, and typically includes many or all of the elements described above relative to the computer. The logical connection between the NIC and the remote computer may include a local area network (LAN), a wide area network (WAN), or both, but may also include other networks. Such networking environments are commonplace in offices, enterprise-wide computer networks, intranets, and the Internet. The remote computer may also represent a web server supporting interactive sessions with the computer; or in the specific case of location-based applications may be a location server or an application server.

In some embodiments, the network interface may use a modem when a broadband connection is not available or is not used. It will be appreciated that the network connection shown is exemplary and other means of establishing a communications link between the computers may be used.

### EXAMPLES

The disclosure is also described and demonstrated by way of the following examples. However, the use of these and other examples anywhere in the specification is illustrative only and in no way limits the scope and meaning of the disclosure or of any exemplified term. Likewise, the disclosure is not limited to any particular preferred embodiments described here. Indeed, many modifications and variations of the disclosure may be apparent to those skilled in the art upon reading this specification, and such variations can be made without departing from the disclosure in spirit or in scope. The disclosure is therefore to be limited only by the terms of the appended claims along with the full scope of equivalents to which those claims are entitled.

### Materials and Methods

Virus, Antigens, Immunogens and Cells.

Stocks of five B virus strains were prepared by infecting African Green Monkey kidney cells (Vero) (catalog no. CCL-81, American Type Culture Collection, Manassas, Va.) as previously described for the B virus laboratory strain E2490 (12). The following B virus strains were used in this study: The E2490 laboratory strain derived from rhesus macaque (Macaca mulata), a generous gift from the late Dr RN Hull, Eli Lilly, Indianapolis Ind.; A clinical isolate, RRn6, isolated from a rhesus macaque (JKH lab); Strain Pmn1, isolated from a pigtail macaque (Macaca nemistrina) (JKH lab); Strain CQ8166 isolated from a cynomolgous macaque (Macaca fascicularis) (JKH Lab); Strain 10R a B virus isolated from a Japanese macaque (Macaca fuscata) (JKH lab). Propagation and harvesting of B viruses were performed in the Viral Immunology Center's BSL4 Laboratory in accordance with the fifth edition of BMBL and the federal regulations (3).

Stocks of other simplexsviruses, HVP2 (EOUP-16) from baboons (Papio spp), HVL (isolated by JKH lab) from a langur monkey (Presbytis spp), HVM (isolated from a sooty mangabeys (Cercocebus atys) (JKH lab), SA8 (B264) isolated from African Green Monkeys (Cercopithecus aethiops), HSV-1 (KOS), and HSV-2 (186), were prepared similarly in Vero cells under standard BSL2 conditions as previously described (1, 12). All viruses were grown in similar and standardized procedures. Infectivity titers determined by a standard plaque assay ranged between 10̂9 and 10̂10 plaque forming units per milliliter (PFU/ml). Virus-antigens for ELISA were prepared by the Tween/DOC detergent solubilization technique. Detergent treatment releases structural and nonstructural antigens and renders them available for interacting with antibodies. The solibilization is also known to inactivate herpes and other lipid enveloped viruses (25, Katz et al, unpublished results). The technique was carried out essentially as previously described (12, 24), except that the picric acid step was omitted. The inventors have found that the addition of picric acid was neither necessary for the adsorption step, nor was it needed for virus inactivation. In fact, it was observed that the addition of picric acid during the antigen adsorption step lowered the ELISA sensitivity for most monoclonal antibodies, probably because its denaturation effect (Katz et al, unpublished results). Virus infected Vero cells grown in 980 cm² roller bottles were harvested 18 to 24 h after observation of nearly 100% cytopathic effect and resuspended in sterile, deionized water containing a protease inhibitor cocktail (Complete Protease Inhibitor, Roche, Indianapolis, Ind.). After disrupting the infected cells by 3 cycles of freeze-thawing, antigens were solubilized by using the detergents Tween-40 and sodium deoxycholate (Sigma Chemical, St. Louis, Mo.), each at a final concentration of 1%. Control antigens for ELISA were prepared from lysates of passage-matched uninfected Vero cells (UN antigen) as described for Virus-antigens (1,12).

Antigens for WBA were prepared by adding 1% SDS to pellets of B virus-infected cells immediately after harvesting (12).

Antigen preparations were assayed for protein concentration, assessed against previous antigen and antibody lots and stored at −70° C. or lower temperatures.

Inactivated B virus immunogens were necessary for the production of mouse monoclonal antibodies.

Preparation of B Virus Immunogens

B virus immunogens where prepared from three B virus strains: rhesus B virus E2490 (Rh-BV), cynomolgus B virus CQ8166 (Cyno-BV) and pigtail B virus Pmn1 (PT-BV).

Mouse 3T3 fibroblast cell line (originated from BALB/c mice) were grown in two 850 cm² roller bottles in DMEM high glucose supplemented with 10% FBS, 200 mM L-glutamine and antibiotics (Pen/Strep) at 37° C. Confluent cell monolayers (95%) in the roller bottles were infected with either one of the B virus strains (5 MOI) and maintained in DMEM high glucose supplemented with 1% FBS and antibiotics. The infected cells were incubated for 24 hrs at 34° C., scraped into the media and centrifuged at 1500 rpm for 10 min. Cell pellets were resuspended in 4.5 ml of sterile ultrapure water. The suspension was then sonicated using Sonics Vibra Cells Sonicator at 75% Amp for 5 min. Cell debris was removed by centrifugation (1500 rpm/10 min) and the virus suspension (about 5 ml) was saved. The virus titers of the suspensions for all three B virus strains, as determined by the plaque assay in Vero cells were ˜2×10⁷ PFU/ml. For inactivation of the B virus strains we have used a “Psoralen-BSPL Photoinactivation Technique” in which psoralen and irradiation by Broad Spectrum Light Pulses (BSPL) are combined. Unlike detergent inactivation the Psoralen photoinactivation damages virus nucleic acids and therefore potential viral DNA replication in the inoculated mice would be most unlikely (26, 27, 28).

The Psoralen-BSPL Photoinactivation Technique (Psoralen-BSPL)

The technique was described in detail in WO/2011/084748. Briefly, each of the B virus preparations (1.4 ml) was diluted 1:5 in sterile ultrapure water to a total volume of 7 ml. Seventy μl of 2 mg/ml Psoralen (4-Aminomethyl-trioxsalen hydrochloride, # A4330, Sigma) were added to the virus suspension resulting in a final concentration of 5 μg/ml. The virus-Psoralen mixture was transferred in 1 ml portions to polyethylene tubings (Polyethylene (Low Density) polytubing # S-3520, 1″ wide, 2 Mil Poly Tubing Roll, ULINE, Atlanta Ga.) that were heat sealed at one end. After transferring the virus to the tubing the other end was heat sealed at a distance of 5 cm. After sealing, the tubing was inserted in a 3 MIL thick polyethylene bag and seal both ends. This step was used to increase the biosafety of the procedure.

The outer part of the second sealed tubings was decontaminated by submersion in a bottle containing CIDEX (activated glutaraldehyde solution) for 15 min. The outside of the bottle was decontaminated by submersion in the CIDEX dunk tank for 15 min. The CIDEX was removed from the bottle and the bottle and tubings were transferred to a quaternary ammonium dunk tank through which they were removed from the BSL4 glove cabinets and then transferred to the BSL3 laboraory. The tubings were then rinsed individually with 70% alcohol. For BSPL exposure, each of the tubings were placed on a flat bed of ice on a tray and inserted into the irradiation chamber of the SteriPulse-XL device (RS-3000C, Xenon Corp.). The distance of the ice surface from the lamp window was 8 shelves (4.26 inches). Each of the virus containing tubings was thus exposed to 12 pulses/4 seconds of BSPL that sums up to a total energy of 5.4 jouls/cm². Following irradiation the content of the individual tubings were pooled and tested for the presence of residual B virus by an infectivity test in Vero cells, for DNA damage by PCR, and for antigenicity by ELISA.

Infectivity Test in Vero Cells for Validating the Inactivation of B Virus

500 μl of the pooled B virus suspension were tested for infectivity in Vero cell monolayers grown in 6 well plates. The cultures were observed microscopically for B virus cytopathic effect (CPE) for 48 hrs. If no CPE developed, cells from the virus infected well were then scraped and transferred to another well containing Vero cells for another 48 hours. The Psoralen-BSPL treated virus batch was considered non-infective if no CPE was observed after replating.

Testing Psoralen-BSPL Induced DNA Damage by PCR

A BV gB primer set that amplifies a 1.3 kb fragment of B virus was used. The PCR reaction was performed by using the PCR HotStar Kit (Qiagen) and 3 μl of purified DNA in 20 μl volume. The amplification was performed on ABI Thermocycler 9600 using the following cycling conditions: 15 min 95° C. and 35 two step cycles of 20 sec at 95° C. and 40 sec at 65° C. Then the PCR reaction products were run on 1% agarose gel along with the DNA marker to determine the presence of the PCR fragment of the expected size. The absence of the PCR fragment after amplification implied that DNA in the sample was damaged and could not be replicated.

Testing Antigenicity of the Psoralen-BSPL Inactivated B Virus by ELISA

B virus immunogens that were negative by the infectivity test and by PCR were tested by ELISA essentially as previously described (12). Briefly, the immunogens were adsorbed to wells of a 96-well microplate at a final dilution of 1:100 in PBS, incubated for 1 hour at 37° C., washed 3 times with borate-buffered saline (BBS) containing 0.01% Tween 20, and blocked with Blotto (2.5% nonfatmilk and 2.5% liquid gelatin in BBS). Six, 3-fold dilutions of a rhesus anti B virus positive standard serum starting from 1:50, were then incubated in the wells. After 3 cycles of washes, anti-human IgG alkaline phosphatase conjugate was added to the wells for 1 hour at 37° C. Wells were washed again and a dNPP substrate was added for 30 min at room temperature. Color intensity was then monitored in an ELISA-Reader at a wavelength of 405 nm.

Serum Sample Collection

Nonhuman primate sera and human sera were submitted to the National B Virus Resource Center (Atlanta, Ga.) as part of a routine surveillance or as a result of a human injury associated with a specific nonhuman primate. All sera were obtained from the National B Virus Resource Center after being tested for antibodies as previously described (12, 13). In this study B virus positive and negative sera from rhesus and cynomolgus macaques were used. In addition, the inventors used human sera that tested antibody negative for all simplexviruses, and sera that were antibody positive for B virus, HSV-1 and HSV-2.

Preparation of Standard BV-Positive and-Negative Pooled Macaque Sera

BV-negative macaque serum pools were prepared from at least 100 macaque sera that were determined as negative by tELISA. BV-positive macaque serum pools were prepared from at least 100 macaque sera that were determined as positive by tELISA. Sera were considered to be positive for antibodies to BV if the positive:negative (P:N) ratio was 3.5 or greater; negative sera fell below this threshold. To determine the P:N ratio of a sample, the OD405 of the 1:5-diluted sample in the BV-coated well was divided by the OD405 of the UN-coated well.

The titer, expressed in ELISA units (EU), of each new positive standard pool of sera was predetermined in a quality-control experiment in which at least 12 replicate dilution series were tested in wells coated with the whole-BV lysate and in control wells coated with lysates from uninfected cells. The inventors defined 1 EU as the reciprocal serum dilution of the standard positive serum at a cutoff value. The cutoff value was arbitrarily defined as 2 times the average OD405 values obtained from UN-coated wells that reacted with the first positive serum pool dilution that generated an OD405 of 1.0 or greater in BV-coated wells (12).

The Production of MABs to B Virus Antigens

The production of MABs to B virus was outsourced to the monoclonal antibody facility (MAF) at the University of Georgia (UGA). The work at the MAF-UGA facility included the preparation of hybridomas and one liter portions of supernatants containing MABs. Hybridomas were generated by inoculating mice with B virus Psoralen-BSPL inactivated immunogens that were prepared by us at GSU. One hybridoma clone was obtained by inoculating the mice with a recombinant glycoprotein B preparation. In addition, B virus antigen and control antigen coated microplates that enabled selection of B virus specific hybridomas by ELISA were provided. Additional MAB characterization experiments and phage display selection experiments were performed at GSU.

Immunization of Mice

According to the MAF-UGA protocol mice to be inoculated with B virus immunogens (Rh-BV, Cyno-BV and PT-BV), were first pretreated at time zero with Complete Freund's Adjuvant (CFA). Two days later they were injected with 1:10 dilution containing approximately 10̂6 PFU/ml of B virus before inactivation. Booster immunizations (immunogens without adjuvant) were then given at 3, 6, 9 and 12 weeks after first pretreatment immunization. Mice were bled for testing before immunization and 7 days after each booster injection. In addition, an experiment was set up in which the efficacy of CFA was compared to Dimethyl Dioctadecyl Ammonium Bromide (DDA) to CFA. DDA is known as a potent adjuvant that promotes both cellular and humoral immunity to lipid envelope viruses (29). One group of 2 mice was immunized with the Cyno-BV and PT-BV immunogens and CFA and at the same time another group of 2 mice were immunized with a mixture of the same immunogens and DDA. The immunization and bleeding schedules for the DDA group was similar to the schedule of the CFA group.

### Testing the Reactivity of MABs by ELISA and by a Polyclonal-MAB Competition ELISA (PAB-MAB CE)

The antibody activity of the MABs to B virus strains and to a panel of simian and human simplexviruses was tested by ELISA as previously described (12) except that the conjugate used was Goat anti Mouse IgG—Alkaline Phosphatase instead of Goat anti Human IgG—Alkaline Phosphatase. For validation purposes we also performed a competition ELISA test in which primate sera containing polyclonal antibodies (PABs) were used to compete for binding to immobilized B virus antigen (PAB-MAB CE). In this test PAB containing sera from macaques or humans were first incubated for 1 hr at 37° C. in microplate wells that were precoated with B virus antigen. After washing the wells the MAB of interest was added for another 1 hr incubation at 37° C. After additional washes the anti mouse IgG conjugate was added and incubated (1 hr at 37° C.). The wells were then washed again, incubated at room temperature with the substrate and results were read in the ELISA reader.

Western Blot Analysis (WBA)

WBA was performed according to standardized, CLIA approved, diagnostic lab protocols essentially as previously described (12) Briefly, lysates of BV infected cells were diluted in SDS disruption buffer (4% SDS, 4% 2-mercaptoethanol, and10% glycerol in 50 mMTris-HCl, pH 6.8), boiled for 3 min, and loaded on discontinuous gradient gels (8% to 16%). Proteins were transferred from the gel to nitrocellulose membranes (0.45 μm). After transfer, membranes were cut into 3×95-mm strips, which were blocked with Bllotto and incubated with test sera diluted in the same buffer (1 h at 37 ° C.). Antibodies in monkey or human sera were detected by incubating strips in biotinylated goat anti human IgG (1 h at 37° C.), followed by incubation with avidin-alkaline phosphatase (30 min at room temperature) and an appropriate alkaline phosphatase substrate (incubated until sufficient color developed). Antibody reactivity to mouse MABs were detected by incubating strips with goat anti mouse IgG (1 h at 37° C.), followed by incubation with avidin-alkaline phosphatase (30 min at room temperature) and an appropriate alkaline phosphatase substrate (incubated until sufficient color developed). Sera or MABs were compared to controls that consisted of BV positive and negative pooled macaque sera or MABs, and to marker proteins for estimation of molecular weights.

Immunoprecipitation

Two kits for immunoprecipitation were used in our experiments: One was purchased from Santa Cruz Biotechnology, Inc. Dallas, Tex., and the second from Thermo Scientific Pierce. The techniques were carried out basically as instructed by the manufacturers' protocols.

Mass Spectrometry for Protein Identification (MSA)

The MSA was carried out by Dr. Hyuk-Kyu Seoh, at the core facility of the department of biology, core facility at GSU.

Protein bands of interest were excised from the gel. The excised gel pieces were washed first with dd-H₂O and subsequently with washing solution I (50% Methanol, 25 mM Ammonium bicarbonate, pH 8.3), and washing solution II (50% Acetonitrile, 25 mM Ammonium bicarbonate, pH 8.3). The washed gel pieces were finally dehydrated with 100% acetonitrile and dried under speed-vac.

The dried gel pieces either immediately underwent Trypsin digestion or were kept at −80° C. until they were treated with trypsin for the mass spectrometry peptide analysis. In brief, the gel pieces were incubated with appropriate amount of Trypsin (Modified Trypsin Gold, Promega, Madison, Wis.) in proteomax surpectant (Promega, Madison, Wis.) at 37° C. for 2˜3 hours. After incubation, the digested peptides were extracted with 2.5% of Trifluoroacetic acid. The extracted peptides were further purified and concentrated by ZipTip, a C18 micro-reverse phase resin tip (Millipore, Billerica, Mass.) acceding to the manufacturer's protocol.

Extracted peptides were then analyzed by 4800 MALDI Tof/Tof tandem mass spectrometer (AB Sciex, Framingham, Mass.) with MS/MS tandem mode. Protein identifications were carried out by Mascot search engine (Matrix Science Inc, Boston, Mass.) against Swiss pro or NCBI protein database.

Epitope Mapping Using Phage Display Libraries

Phage display random 7-mer, 7-mer cyclic and 12-mer peptide libraries were purchased from “NEW ENGLAND BioLabs Inc., Ipswich, Mass. The phage display epitope mapping technique is based on a M13 phage vector, modified for pentavalent display of peptides as N-terminal fusions to the minor coat protein pIII. The selection of the different variants was done according to the manufacturer's protocol “Surface Panning Procedure (Direct Target Coating)” using the in vitro technique named “panning” (32). Briefly, panning was carried out by incubating a library of phage-displayed peptides on wells of a 96-well microplate, precoated with the target MAB. Unbound phages were washed away and the remaining bound phages were eluted with glycine buffer pH 2.2, neutralized in Tris buffer pH 9.0, amplified in the E. Coli bacteria, and purified by PEG-NaCl precipitation and centrifugation. After an additional binding, elution and amplification cycle, a third panning cycle was done to enrich the pool in favor of specific binding peptide sequences. The phage eluate of the third cycle was then tittered in a LB/IPTG/Xgal plate and after 24 hours incubation at 37° C. the titer of the phage colonies (clones) was determined by counting blue plaques. Individual picked clones from the LB/IPTG/Xgal agar plate were tested by ELISA for specific binding to the MAB as follows:

Fifty microliters of the MAB at 10 μg/ml in bicarbonate buffer were added to a 96-well microplate and incubated for 60 min at room temperature (R/T). The wells were then blocked with BLOTTO and by incubation at at 37° C. for 60 min.

Fifty μl of the enhanced individual clones picked from titration plates and enriched were incubated in the MAB coated wells at a 1:10 in TBST 0.1%, for 1 hr at R/T. After 3 washes with BBST, the HRP-anti M13 Monoclonal Conjugate. (diluted 1:5000 in TBST 0.1%) is added for 1 hr at R/T. After additional 3 washes with BBST, 50 μl of the TMB substrate is added for 15-20 min. The reaction is stopped by adding 50 μl of 1 M sulfuric acid and the results are read in a microplate-ELISA reader at 450 nm.

Positive phage clones were then selected for DNA sequencing that was performed by Ms. Ping Yang at the core facility of the department of biology, core facility at GSU.

Peptide Microarray Screening

The peptide array screening was performed at the Department of Chemical Biology, Helmholtz Centre for Infection Research, Braunshweig, Germany as part of a collaborative study with Dr. Ulrike Beutling and Prof. Mark Broenstrup (MTA 14-55). MABs were sent to Germany for screening on an overlapping microarray of peptides based on gB and gD of the B virus E2490 strain. The technique was carried out as previously described (34).

Neutralization Method:

Vero cell monolayers were grown to confluency in a 48 well plate. Monoclonal antibody 3-fold dilutions, 1:2 through 1:54 and Rhesus positive serum 3-fold dilutions, 1:10 to 1:810 were prepared in the BSL-2. For negative controls, rhesus negative serum and mouse normal serum was diluted and added to virus to achieve a final dilution of 1:10.

Some MABs that were not concentrated or purified were tested at a 1:2 dilution and others that were concentrated or purified were tested at 3 fold dilutions (1:2 to 1:54). The 48-well Vero cell plate and the serum dilutions were transferred to the BSL-4 to for the addition of 25 μl of t 50 PFU (plaque forming units) B virus to 25 μl of the diluted serum. This 50 μl virus+serum mixture was incubated at 37° C. incubator for 1 hour. After incubation 50 μl of virus+serum mix was added to each well of a 48 well plate and incubated for 1 hr at 37° C. for virus adsorption. After an additional 1 hour incubation, the virus+serum inoculum was removed and methyl cellulose was added to each well. The plate was incubated for 48 hrs in a 37° C. incubator to allow for plaque formation. After 48 hours, methylcellulose from each well was removed and the wells were washed with PBS and fixed with 100% methanol. The plate was dunked out of BSL-4, washed, and then the cell cultures in the wells were stained with crystal violet (0.2%) to count the number of plaques.

### Results and Discussion

Characterization of MABs

Eighteen MAB hybridomas were generated at MAF-UGA. Different immunogens and adjuvants were used as shown in Table 1.

TABLE 1
MABs produced by inoculating mice with different immunogens: Rec-gB (B
virus recombinant glycoprotein B), Rh-BV (E2490 laboratory strain), PT-BV (Pmn1, isolated
from a pigtail macaque), Cyno-BV (CQ8166 isolated from a cynomolgous macaque).

| | | | | Antibody | Method for |
|---|---|---|---|---|---|
| Serial | Immunogen | MAB | Class or | Reactivity | detecting MAB |
| Number | (Adjuvant) | Name | subclass | to: | reactivity |
| 1 | Rh-BV, Rec gB | 7H1.G5 | IgG1 | gB | Rec-ELISA, IP/MSA |
| | (CFA) | | | | |
| 2 | Rh-BV (CFA) | 12F5.C1 | IgG1 | VP13/14 | WBA, IP/MSA |
| 3 | Rh-BV (CFA) | 18D10.F2.A4 | IgA | gB | Rec-ELISA, IP/MSA |
| 4 | Rh-BV (CFA) | 2G12.D12.D4 | IgG2a | gD | Rec-ELISA, WBA, |
| | | | | | Peptide Microarray |
| 5 | Rh-BV (CFA) | 6E10.D7 | IgG2b | gD | Rec-ELISA, WBA, |
| | | | | | Peptide Microarray |
| 6 | Rh-BV (CFA) | 5D10.C9 | IgG1 | gB | Rec-ELISA |
| 7 | Rh-BV (CFA) | 5E10. C10 | IgG1 | gB | Rec-ELISA |
| 8 | Rh-BV (CFA) | 7G9.E3 | IgG1 | gB | Rec-ELISA |
| 9 | Rh-BV (CFA) | 12G9.G5 | IgG2a | gE and gI | IP/MSA |
| 10 | Rh-BV (CFA) | 10F9.F1 | IgG2a | Not | ELISA |
| | | | | specific | |
| 11 | Rh-BV (CFA) | 7F7.G7 | IgG2b | gB | Rec-ELISA |
| 12 | PT-BV (DDA) | 5A2 | IgG1 | gB | Rec-ELISA |
| 13 | PT-BV (DDA) | 4E11 | IgG1 | gB | Rec-ELISA |
| 14 | Cyno-BV (DDA) | 9F1 | IgG2a | gB | Rec-ELISA, WBA, |
| | | | | | Peptide Microarray |
| 15 | Cyno-BV (DDA) | 3H6 | IgG2a | gB | Rec-ELISA, WBA, |
| | | | | | Peptide Microarray |
| 16 | Cyno-BV (DDA) | 7G2 | IgG2a | gB | Rec-ELISA, WBA, |
| | | | | | Peptide Microarray |
| 17 | Cyno-BV (CFA) | 7G8 | IgG2a | gB | Rec-ELISA |
| 18 | Cyno-BV (CFA) | 1G3 | IgG2a | gB | Rec-ELISA |

It can be seen from Table 1 that the majority of MABs reacted to the gB protein, two MABs reacted to gB one MAB reacted to the tegument protein gVP13/14 and one reacted to gI and gE. One of the 18 MABs reacted nonspecifically to the uninfected (UN) control and therefore was not further characterized.

The seventeen B virus specific MABs, listed in Table 1, can be grouped in five categories based on their specificity to simplexviruses:
1. MABs that are selectively specific for Rh-BV and Jap-BV strains, and do not react with PT-BV or Cyno-BV or to any other simplexviruses (Jap-BV strain 10R isolated from a Japanese macaque) (Table 2).
2. MABs that react with all four tested B virus strains (Rh-B, Jap-BV, PT-BV and Cyno-BV), and do not react with other simplexviruses (Table 3).
3. MABs that react with B virus recombinant glycoprotein D (BV-gD) but does not react with HSV-1 recombinant glycoprotein D (HSV1-gD) (Table 4).
4. MABs that react with B virus and other simian simplexviruses, do not react with human HSV-1 and HSV2, react with BV-gB and not with HSV1-gB (Table 5).
5. MABs that react with B virus and other simian simplexviruses, react less with human HSV-1 and HSV2, react with BV-gB and also react HSV1-gB (Table 6).

The Category 1 MABs, 12F5.C1 and 12G9.G5, described in Table 2, are of particular interest since they recognized only two out of the four B virus strains tested (Rh-BV and Jap-BV). Other investigators have shown that the different B virus strains have distinct genotypes (23,) and that a cynomolgous immunogen resulted in a MAB that was specific for the Cyno-BV strain and did not recognize the Rh-BV strain (21, 33). Polyclonal-Monoclonal competition experiments (PAB-MAB CE) revealed that macaque sera containing anti B virus antibodies prevented the binding of MAB 12G9.G5 and 12G9.G5 to B virus. However, cynomolgus sera containing anti B virus antibodies did not block the binding of the MABs to B virus. In addition antibodies to HSV-1 and HSV-2 were also incapable of blocking these MABs. When human sera from B virus antibody positive individuals were tested it was found that only 2 out of 18 sera were able to block the binding of the MABs to B virus. These two individuals were known to be infected with the Rh-BV strain while the B virus strain that infected the others was not known. Also, both individuals were symptomatic while the other were asymptomatic. It is possible that sera from seropositive humans that did not compete in the PAB-MAB CE test were infected with a cyno-BV or PT-BV strain and therefore could not compete with these specific MABs. As suggested by Smith et al (23) it is possible that the different B virus strains may also differ in their pathogenicity.

The results herein demonstrate that the 12F5.C1 and 12G9.G5 MABs may serve as a valuable diagnostic reagent for the specific identification of Rh-BV or Jap-BV antibodies in macaque and human sera. Also, in preliminary results using an immunofluorescent test, the results herein demonstrated that these antibodies may be used as a reagent for identification of B virus isolates directly in infected tissues or cell cultures.

Immunoprecipitation in combination with mass spectrometry analysis (IP/MSA) revealed that the MAB 12F5.C1 recognizes a virus internal tegument protein VP13/14 and that the 12G9.G5 MAB recognized two glycoproteins gE and gl. The 12G9.G5 MAB did not react against the gl and gE glycoproteins in ELISA. The gI and gE proteins are known to complex during the process of virus infection and it is therefore, possible that the reaction is against one of the proteins or against the junction site of these 2 proteins. More studies are needed for understanding this result. However, both MABs 12F5.C1 and 12G9.G5 may be used in the future as a research tool for studying the molecular events of host virus interactions.

TABLE 2
MABs that are selectively specific for Rh-B virus and Jap-BV strains, and do
not react with PT-BV or Cyno-BV or to any other simplexviruses. ELISA results were
obtained from duplicate independent experiments and presented as percent of reactivity
relative to the Rh-BV (lab strain) ± standard deviation. Neg = Negative; Pos = Positive.

| | | MAB (Name) | |
|---|---|---|---|
| | | 12F5.C1 | 12G9.G5 |
| | | Sub-Class | |
| | | IgG1 | IgG2a |
| Tests | Tested against: | Test Result | Test Result |
| ELISA | UN-Control | Neg | Neg |
| | Rh-BV (lab strain) | 100 | 100 |
| | Rh-BV isolate | 109.4 ± 2.0 | 74.4 ± 13.2 |
| | Cyno-BV | 0.4 ± 0.0 | 8.7 ± 4.2 |
| | PT-BV | 0.5 ± 0.3 | 0.4 ± 0.0 |
| | Jap-BV | 115.9 ± 22.6 | 119.3 ± 22.1 |
| | HVP-2 | Neg | Neg |
| | SA8 | Neg | Neg |
| | HVL | Neg | Neg |
| | HVM | Neg | Neg |
| | HSV-1 | Neg | Neg |
| | HSV-2 | Neg | Neg |
| | gB | Neg | Neg |
| | gC | Neg | Neg |
| | gD | Neg | Neg |
| | gGm | Neg | Neg |
| | gG1 | Neg | Neg |
| | gG2 | Neg | Neg |
| | gE | Neg | Neg |
| | gI | Neg | Neg |
| WB | BV | Pos (~75 Kd.) | Neg |
| IP/MSA | BV | Pos (72 Kd) | Pos (45 Kd & 60 Kd) |
| PAB-MAB CE | Competing for BV | Pos | Pos |
| Phage Display | Epitope Mapping | + | + |
| | Synthetic Peptides | + | + |
| Peptide-MAB | Competing for BV | + | + |
| CE | | | |

The Category 2 MABs, described in Table 3, are B virus specific anti-gB MABs that reacted with all four B virus strains tested. All these MABs reacted strongly by WBA indicating that they identify linear epitopes. The MABs did not react with any of the other simplexviruses and also they did not react with HSV1-gB. The 1G3.C1 hybridoma is a subclone of 1G3 hybridoma and therefore the 1G3 and the 1G3.C1 MABs are identical. The 9F1, 3H6 and 7G2 MABs are probably also identical because all of them reacted in the peptide microarray analysis to the same overlapping amino acid residues (Table 7). Interestingly, in spite of their specificity to B virus, the IG3, 9F1, 3H6 and 7G2 MABs were competed by anti-HSV1 and anti- HSV2 positive sera in the PAB-MAB CE test. It is possible that this result is caused by steric hindrance where the PAB antibody tested may be binding to neighboring cross-reactive epitopes, thereby blocking the reaction of the MAB to its specific epitope. The identification of the linear peptides that are located on the ectodomain portion of the BV-gB protein is of utmost importance. The stretch of six peptides that were likely essential for binding the MABs in the peptide array represent a specific B virus epitope that is not present on the HSV1-gB. It is possible that this peptide, once synthesized, will enable specific diagnosis of B virus infections in humans.

TABLE 3
MABs that react with all four tested B virus strains (Rh-B, Jap-BV, PT-BV
and Cyno-BV), and do not react with other simplexviruses. ELISA results were obtained
from one single experiment and presented as percent of reactivity
relative to the Rh-BV (lab strain).

| | | | | | | |
|---|---|---|---|---|---|---|
| | MAB (Name) | 1G3 | 1G3.C1 | 9F1 | 3H6 | 7G2 |
| | Sub-Class | IgG2a | IgG2a | IgG2a | IgG2a | IgG2a |
| | | Test | Test | Test | Test | Test |
| Tests | Tested against | Result | Result | Result | Result | Result |
| ELISA | UN - Control | Neg | Neg | Neg | Neg | Neg |
| | Rh-BV (lab strain) | 47.5 | 45.7 | 97.3 | 99.6 | 89.3 |
| | Rh-BV isolate | 28.1 | 29.4 | 44.2 | 49.8 | 47.6 |
| | Cyno-BV | 100 | 100 | 100.0 | 100.0 | 100.0 |
| | Pig-Tail M. BV | 52.6 | 53.6 | 46.0 | 49.9 | 48.4 |
| | Jap. Mount. M. BV | 67.8 | 50.9 | 62.8 | 65.9 | 62.2 |
| | HVP-2 | 2.8 | 1.5 | 1.7 | 1.7 | 2.3 |
| | SA8 | 2.8 | 0.6 | 1.0 | 1.1 | 0.9 |
| | HVL | 2.8 | 2.4 | 1.1 | 1.8 | 2.3 |
| | HVM | 2.5 | 1.0 | 1.5 | 2.0 | 0.5 |
| | HSV-1 | 12.8 | 10.2 | 0.8 | 0.9 | 1.0 |
| | HSV-2 | 5.5 | 3.1 | 1.0 | 3.0 | 1.4 |
| | gB (BV) | Pos | Pos | Pos | Pos | Pos |
| | gC (BV) | Neg | Neg | Neg | Neg | Neg |
| | gD (BV) | Neg | Neg | Neg | Neg | Neg |
| | gGm (BV) | Neg | Neg | Neg | Neg | Neg |
| | gB (HSV1) | Neg | Neg | Neg | Neg | Neg |
| | gG1 (HSV1) | Neg | Neg | Neg | Neg | Neg |
| | gG2(HSV1) | Neg | Neg | Neg | Neg | Neg |
| | | Pos | | Pos | Pos | Pos |
| | | (120 | Pos (120 | (120 | (120 | (120 |
| WB | BV | Kd) | Kd) | Kd) | Kd) | Kd) |
| PAB-MAB CE | Competing for BV | Pos | Pos | Pos | Pos | Pos |
| Phage Display | Epitope Mapping | NT | NT | NT | NT | NT |
| Peptide | | NT | NT | Pos | Pos | Pos |
| microarray | | | | | | |

NT = Not Tested.
Neg = Negative;
Pos = Positive.

The Category 3 MABs, described in Table 4, are B virus specific anti-gD MABs. For an unknown reason they react very poorly to the whole B virus antigen in ELISA although they are highly reactive by WBA and by recombinant ELISA against the BV-gD recombinant protein. It could be that the reaction is dependent on revealing the epitope by the denaturation process of the virus antigen that is part of the WBA. They however, do not react with HSV1-gD. The 2G12.D12.D4 and 6E10.D7 MABs are probably reacting with the same epitope because both of them reacted in the peptide microarray analysis to the same overlapping amino acid residues (Table 7). The identification of the linear peptides that are located on the ectodomain portion of the BV-gD protein was very important. The stretch of six peptides that were likely essential for binding the MABs in the peptide array represent a specific B virus epitope that is not present on the HSV1-gD. It is possible that this peptide, once synthesized, will enable specific diagnosis of B virus infections in humans. Epitope mapping by the phage display techniques resulted in mimitopes but their evaluation is not completed. NT=Not Tested. Neg=Negative; Pos=Positive.

TABLE 4
MABs reacting with recombinant protein glycoprotein D (gD).

| | | MAB (Name) | |
|---|---|---|---|
| | | 2G12.D12.D4 | 6E10.D7 |
| | | Sub-Class | |
| Characterization | | IgG2a | IgG2b |
| Tests | Tested against | Test Result | Test Result |
| ELISA | UN-Control | Neg | Neg |
| | Rh-BV (lab | 1+ | 1+ |
| | strain) | | |
| | Rh-BV isolate | NT | NT |
| | Cyno-BV | NT | NT |
| | Pig-Tail M. BV | NT | NT |
| | Jap. Mount. M. | NT | NT |
| | BV | | |
| | HVP-2 | NT | NT |
| | SA8 | NT | NT |
| | HVL | NT | NT |
| | HVM | NT | NT |
| | HSV-1 | NT | NT |
| | HSV-2 | NT | NT |
| | gB* | Neg | Neg |
| | gC* | Neg | Neg |
| | gD* | Pos | Pos |
| | gGm* | Neg | Neg |
| | gG1** | Neg | Neg |
| | gG2*** | Neg | Neg |
| WB | BV | +(50 Kd) | +(50 Kd) |
| PAB-MAB CE | Competing for | ? | ? |
| | BV | | |
| Phage Display | Epitope Mapping | + | + |
| | Synthetic | + | + |
| | Peptides | | |
| | Competing for | Pos | Pos |
| | BV | | |
| Peptide | | Pos | Pos |
| microarray | | | |

The Category 4 MABs, described in Table 5, are all positive against BV-gB and negative against HSV1-gB by the recombinant ELISA. The 5E10.C10 and the 7G8 MABs interact with all B virus strains while the 7F7.G7 MAB does not react against the PT-BV strain. All three MABs react to different degrees with the simian simplexviruses and do not react with HSV-1 and HSV-2. In spite of the non-reactivity to the human simplexviruses (HSV-1 and HSV-2) these MABs are competed efficiently by anti-HSV-1 and anti-HSV-2 positive human sera. As explained for the Category 2 MABs. It is possible that this result is caused by steric hindrance where the PAB anti-HSV antibody may be binding to neighboring cross-reactive epitopes, thereby blocking the reaction of the MAB to its specific epitope. All three MABs were negative by WBA, suggesting that they recognize conformational epitopes. The MABs as expected did not show any binding to the linear sequences provided by the peptide microarray technique. Epitope mapping using the phage display technique was performed for the 7F7.G7 MAB but the evaluation studies for the selected peptides were not completed.

TABLE 5
MABs that react with B virus and other simian simplexviruses, do not react
with human HSV-1 and HSV2, react with BV-gB and not with HSV1-gB. NT = Not Tested.
Neg = Negative; Pos = Positive.

| | | MAB (Name) | | |
|---|---|---|---|---|
| | | 5E10.C10 | 7F7.G7 | 7G8 |
| | | Sub Class | | |
| Characterization | Tested | IgG1 | IgG2b | IgG2a(k) |
| Tests | against | Test Result | Test Result | Test Result |
| ELISA | UN-Control | Neg | Neg | Neg |
| | Rh-BV (lab | 100 | 100 | 48 |
| | strain) | | | |
| | Rh-BV | 113.2 | 106.3 ± 15.6 | 33.4 |
| | isolate | | | |
| | Cyno-BV | 145.9 | 55.8 ± 22.9 | 100 |
| | PT-BV | 75.5 | 3.8 ± 4.7 | 64.8 |
| | Jap-BV | 104.5 | 80.5 ± 3.1 | 60.8 |
| | HVP-2 | 117.6 | 90.9 | 44.1 |
| | SA8 | 31.5 | 11.3 | 18.8 |
| | HVL | 55.6 | 39.7 | 35.9 |
| | HVM | 60.4 | 16.8 | 33.8 |
| | HSV-1 | 0.76 | 4.6 ± 3.9 | 1.2 |
| | HSV-2 | 0.82 | 2.6 ± 0.8 | 4.9 |
| | gB (BV) | Pos | Pos | Pos |
| | gC (BV) | Negative | Negative | Negative |
| | gD (BV) | Negative | Negative | Negative |
| | gGm (BV) | Negative | Negative | Negative |
| | gB (HSV1) | Neg | Neg | Neg |
| | gG1 (HSV1) | Neg | Neg | Neg |
| | gG2( HSV1) | Neg | Neg | Neg |
| WB | BV | Neg | Neg | Neg |
| IP | BV | Positive on | Positive on | NT |
| | | BV, Neg on | BV, Neg on | |
| | | HSV | HSV | |
| PAB-MAB CE | Competing | Pos | Pos | Pos |
| | for BV | | | |
| Phage Display | Epitope | NT | + | NT |
| PAB-MAB CE | Mapping | | | |
| | Synthetic | NT | NT | NT |
| | Peptides | | | |
| | Competing | NT | NT | NT |
| | for BV | | | |
| Peptide microarray | | Neg | Neg | Neg |

The Category 5 MABs, described in Table 6, were all positive against BV-gB and also to HSV1-gB by the recombinant ELISA. However, in general, the reactivity to the heterologous HSV1-gB was lower. All seven MABs interact with all B virus strains. All MABs reacted to different degrees with other simian simplexviruses. The reaction to the human simplexviruses, HSV-1 and HSV-2, was significantly lower. The 18D10.F2.A4 MAB exhibited the highest reactivity to these viruses (˜20%). All other MABs showed a very low reactivity ranging from 13.2 to 0.8%. Of the 7 MABs, 5A2 and 4E11 MABs showed the lowest reaction to the heterologous human viruses. These two MABs are the only MABs that were found to possess neutralization activity against B virus in cultures.

All Category 5 MABs were negative by WBA, suggesting that they recognize conformational epitopes. The MABs as expected did not show any binding to the linear sequences provided by the peptide microarray technique. Epitope mapping using the phage display technique was performed for some of the MABs but the evaluation studies of the peptides selected were not completed.

TABLE 6
MABs that react with B virus and other simian simplexviruses, react less
with human HSV-1 and HSV2, react with BV-gB and also react HSV1-gB.

| | | | | | | | |
|---|---|---|---|---|---|---|---|
| | MAB | 18D10.F | 7H1.G5 | 5D10.C9 | 7G9.E3 | 5A2 | 4E11 |
| | (Name) | 2.A4 | | | | | |
| | Sub-Class | IgA | IgG1 | IgG1 | IgG1 | IgG1 | IgG1 |
| | or Class | | | | | | |
| | Tested | | | | | | |
| Tests | against | Result | Result | Result | Result | Result | Result |
| ELISA | UN - | Neg | Neg | NEG | Neg | Neg | Neg |
| | Control | | | | | | |
| | Rh-BV (lab | 100 | 100 | 100 | 100 | 135.8 | 145.3 |
| | strain) | | | | | | |
| | Rh-BV | 110.4 | 92.8 | 105.5 | 97 ± 0.4 | 108.5 | 107.4 |
| | isolate | | | | | | |
| | Cyno-BV | 104.3 | 94.1 | 101.8 | 100.4 ± 3.1 | 127.7 | 131 |
| | PT-BV | 75.1 | 107.8 | 23.4 | 19.6 ± 1.8 | 100 | 100 |
| | Jap-BV | 95.1 | 114.6 | 101.7 | 96.4 ± 0.4 | 115.9 | 121.9 |
| | HVP-2 | 78.6 ± 19.6 | 10.9 | 101.8 | 98.0 | 19.1 | 9.9 |
| | SA8 | 35.4 ± 5.2 | 0.5 | 39.14 | 39.3 | 1.9 | 2.5 |
| | HVL | 63 ± 10.1 | 53.9 | 91.8 | 81.7 | 64.0 | 46.9 |
| | HVM | 59.5 ± 22.5 | 46 | 85.3 | 30.2 | 28.7 | 23.7 |
| | HSV-1 | 22.9 ± 5.6 | 3.7 | 13.2 | 6.9 | 0.8 | 1.3 |
| | HSV-2 | 19.4 ± 8.2 | 4.2 | 12.5 | 3.7 | 1.9 | 2.6 |
| | gB (BV) | Pos | Pos | Pos | Pos | Pos | Pos |
| | gC (BV) | Neg | Neg | Neg | Neg | Neg | Neg |
| | gD (BV) | Neg | Neg | Neg | Neg | Neg | Neg |
| | gGm (BV) | Neg | Neg | Neg | Neg | Neg | Neg |
| | gB (HSV1) | Pos | Pos | Pos | Pos | Pos | Pos |
| | gG1(HSV1) | Neg | Neg | Neg | Neg | Neg | Neg |
| | gG2(HSV2) | Neg | Neg | Neg | Neg | Neg | Neg |
| WB | BV | Neg | Neg | Neg | Neg | Neg | Neg |
| IP | BV | + | + | NT | + | NT | NT |
| PAB-MAB | Competing | + | + | + | + | + | + |
| CE | for BV | | | | | | |
| Phage | Epitope | + | + | + | + | NT | NT |
| Display | Mapping | | | | | | |
| | Synthetic | + | + | + | + | NT | NT |
| | Peptides | | | | | | |
| | Peptide | + | + | + | + | NT | NT |
| | Competing | | | | | | |
| | for BV | | | | | | |
| Peptide | | Neg | Neg | Neg | Neg | Neg | Neg |
| microarray | | | | | | | |

NT = Not Tested.
Neg = Negative;
Pos = Positive.

Results of Epitope Mapping

Epitope mapping was carried out using two techniques, the Phage Display technique and the Peptide Array technique as described in material and methods.

Table 7 contained all the peptides that were identified so far by the epitope mapping techniques. Some of the peptides were not synthesized and others were synthesized and tested for activity.

TABLE 7
Identification of B virus related peptides by Epitope Mapping techniques
using B virus induced MABs for Phage display and Peptide Microarrays, and
Human Sera for Peptide Microarrays.
Peptides discovered by Phage Display

| MAB | | SEQ | | | |
|---|---|---|---|---|---|
| (Subclass) | Peptide | NO: | Name | Consensu | Notes |
| 12F5.C1 | DYWSYWWGGGS | 13 | P1 | 4/10 | Anti- |
| (IgG1) | IDKSEYWTYQLRGGGS | 14 | P4 | 2/10 | Vp13Np14 |
| 18D10.F2.A4 | CKHEPYFHCGGGS | 15 | | 4/18 | Anti-gB |
| (IgA) | | | | | |
| 12G9.G5 | NMANYPLTRPALGGGS | 16 | | (8/2 | Anti-gE/gI? |
| (IgG2a) | | | | | |
| 7H1.G5 | QPWYQVKGGGS | 17 | | 5/10 | Anti-gB |
| (IgG1) | QHSFDHYLPLSPGGGS | 18 | | 7/10 | |
| 7F7.G7 | SEKEPPVGGGS | 19 | | 3/11 | Anti-gB |
| (IgG2b) | YGEPPVANWEGSGGGS | 20 | | 2/8 | |
| 7G9.E3 | CELWPMPFCGGGS | 21 | Cyc-2 | Anti-gB | |
| (IgG1) | CTVYPAAWCGGGS | 22 | Cyc-7 | | |
| and/or | CYGMMMEWCGGGS | 23 | Cyc-4 | | |
| 5D10.C9 | CTLYPLPWCGGGS | 24 | | | |
| | CYPLGLLGCGGGS | 25 | | | |
| | CGIEIRISGGGS | 26 | | | |
| | CTETDPPLCGGGS | 27 | | | |
| | CVPTAPFQCGGGS | 28 | | | |
| | CAPPAPVQCGGGS | 29 | | | |
| | CYGMMTERCGGGS | 30 | | | |
| | CYGMTPERCGGGS | 31 | | | |
| | CDGMTPEQCGGGS | 32 | | | |
| | TETDPPLGGGS | 33 | | | |
| 2G12.D12.D4 | WTDETVP | 34 | P8 | 3/6 | Anti-gD |
| (IgG2a) | CDEGVNGYC | 35 | P9 | 15/1 | |
| 6E10.D7 | CHDLNGVPC | 36 | | 12/2 | Anti-gD |
| (IgG2b) | | | | | |
| 9F1 | ANWSHWEFNRRPGGG | 37 | | 7/17 | Anti-gB |
| (IgG2a) | SWRPGDYGGG | 38 | | 17/1 | |
| | CWRPGDMACGGG | 39 | | | |
| | GNGCHRGDNRRGGG | 40 | | 9/14 | |
| | | | | 7/16 | |
| 4E11 | CYEPFWTNC | 41 | | 1/1 | Anti-gB |
| (IgG1) | | | | | (Neutralizing) |
| 5A2 | ACWAPFWSDC | 42 | | 7/10 | Anti-gB |
| (IgG1) | ACLHLPWSPC | 43 | | 3/10 | (Neutralizing) |

Peptides detected with human sera on gB Micro Array

| | HATR | Suggested Epitope | | | |
|---|---|---|---|---|---|
| | AA-Start- | AA-Start- | Suggested Epitope | SEQ ID | |
| HATR | AA-End | AA-End | AA sequence | NO: | Notes |
| 3 | 58-72 | 58-72 | DDNDGEAGAAPG | 44 | Reacted |
| | | | | | with SZ |
| 10 | 190-213 | 199-204 | KYVRNN | 45 | Reacted |
| | | | | | with |
| | | | | | WC |
| 11 | 226-246 | 232-240 | RGWHTTDLK | 46 | Reacted |
| | | | | | with |
| | | | | | SZ, PB, |
| | | | | | GE, |
| | | | | | DR, |
| | | | | | ZM? |
| 14 | 265-288 | 274-279 | FVLATG | 47 | Reacted |
| | | | | | with |
| | | | | | WC |
| 15 | 292-312 | 298-306 | HTAYAADRF | 48 | Reacted |
| | | | | | with |
| | | | | | SZ, PB |
| 17 | 343-366 | 352-357 | TLTKWQ | 49 | Reacted |
| | | | | | with |
| | | | | | WC, |
| | | | | | WS, |
| | | | | | and GE |
| 19 | 367-387 | 370-384 | SFRFSSSALSTTFTT | 50 | (SJ), |
| | | | | | (GE), |
| | | | | | (EJ) |
| 19 | | 382-390 | FTTNRYEYA | 51 | (GE) |
| | | | | | (PP) |
| 22 | 409-429 | 418-426 | GTHVKVQV | 52 | Reacted |
| | | | | | with |
| | | | | | WS, |
| | | | | | PB, SJ, |
| | | | | | GE |
| 24 | 451-468 | 454-465 | ELLREQERRPGD | 53 | PB, |
| | | | | | BN, |
| | | | | | (YS) |
| 24 | 457-474 | 460-471 | ERRPGDAAATPK | 54 | PB, |
| | | | | | (BM) |
| 25 | 469-483 | 475-483 | DPPDVERIK | 55 | SZ |
| ″2 | | 464-474 + 475-478 | GDAAATPKPSADPP | 56 | SZ |
| | | (464-478) | D | | |
| 27 | 529-552 | 538-543 | ASATVG | 57 | SZ, SJ, |
| | | or 535-546 | or NAIASATVGRRV | 58 | DR, |
| | | | | | ZM, |
| | | | | | BN. |
| 28 | 565-585 | 571-579 | QNSMRVPAR | 59 | SZ, SJ, |
| | | or 571-582 | or QNSMRVPARPGT | 60 | DR |
| 30 | 589-609 | 595-603 | EGGPLVEG | 61 | GE, EJ |
| 32 | 631-645 | 631-645 | GAGYVYFEDYAYS | 62 | (PB) |
| | | | | | HQ |
| 34 | 661-681 | 667-675 | DHEFVPLEV | 63 | GE, |
| | | | | | BL, |
| | | | | | (YS) |
| 40 | 814-834 | 820-828 | ASGGGEEDFD | 64 | WS |
| 42 | 844-864 | 850-864 | RTEHKARKKGTSA | 65 | WS, |
| | | | LL | | WS, |
| | | | | | (PB), |
| | | | | | (SJ) |
| 44 | 880-894 | 880-894 | KPRYSPLGDTDEEE | 66 | (SJ) |
| | | | L | | |

Peptides detected with human sera on gD Micro Array

| | HATR | Suggested | | | |
|---|---|---|---|---|---|
| | AA- | Epitope | | | |
| | Start- | AA-Start- | Suggested Epitope | SEQ | |
| HATR # | AA-End | AA-End | AA sequence | ID NO: | Notes |
| 45a | 1-21 | 7-15 | AVLLSLAVA | 67 | WS |
| 45b | 7-24 | 10-21 | LSLAVALARVPA | 68 | DR |
| 45C | 10-33 | 19-24 | VPAGGG | 69 | (GE) |
| 45 | | 7-24 | AVLLSLAVALARVPA | 86 | |
| Combined | | | GGG | | |
| 47 | 31-45 | 31-45 | RSLTRVNPGRFRGAH | 70 | SZ, |
| | | | | | WC, |
| | | | | | WS, |
| | | | | | (SL), |
| | | | | | PB, |
| | | | | | SJ |
| 48a | 40-57 | 43-54 | GAHLAPLEQKTD | 71 | WC, |
| 48b | 46-63 | 49-60 | LEQKTDPPDVRR | 72 | PB |
| 48c | 55-75 | 61-69 | VYHVQPFVE | 73 | SJ |
| 48d | 67-84 | 70-81 | NPFQTPSVPVAV | 74 | YS |
| 53 | 151-171 | 157-165 | LPRWSFYDN | 75 | GE |
| 54 | 208-231 | 217-222 | TLPLRI | 76 | WC |
| 55 | 223-240 | 226-237 | ACLRGPVFEQGV | 77 | PB |
| 57 | 253-279 | 262-267 or | QAAGWH or | 78 | WC, |
| | | 265-273 | GWHGPKAPF | 79 | PB |
| 59 | 292-306 | 292-306 | ELAPEDEDEQAPGDE | 80 | (PP) |
| 60 | 337-351 | 337-351 | SGHTGAIVGALAGAG | 81 | (SJ) |
| 62 | 374-384 | 374-384 | AAGKHVRLPELLDEG | 82 | (WS), |
| | | | or | | (SJ) |
| | | | ELLDEGPGARRGAP* | 83 | |

*This peptide is part of MATR 18 (refr #34). It is BV specific by sequence, reacts
with humans WC, PB, SJ, GE but reacts also with ann HSV2 positive human (AN).
Peptides detected with MABs on gB Micro Array

| | | Suggested | | | |
|---|---|---|---|---|---|
| | | Epitope | Suggested | | |
| | AA-Start- | AA-Start- | Epitope | SEQ | |
| MAB | AA-End | AA-End | AA sequence | ID NO: | Notes |
| 3H6, 7G2, 9F1, | 451-460 | 460-465 | ERRPGD | 84 | Anti |
| 1G3.C1 | | | | | gB |
| (all IgG2a) | | | | | |

Peptides detected with MABs on gD Micro Array

| | | Suggested | | | |
|---|---|---|---|---|---|
| | | Epitope | Suggested | | |
| | AA-Start- | AA-Start- | Epitope | SEQ | |
| MAB | AA-End | AA-End | AA sequence | ID NO: | Notes |
| 2G12.D12.D4 | 289-312 | 298-303 | EDEQAP | 85 | Anti |
| (IgG2a), | | | | | gD |
| 6E10.D7 (IgG2b) | | | | | |

In this disclosure, the inventors have produced a range of B virus peptide epitopes that may enable the design of novel, specific and sensitive serological immunoassays for use in multiple formats for detecting B virus infections in macaque and humans. The strategy for achieving this goal was production of B virus specific MABs and their use for identifying B virus specific peptides (epitopes and or mimitopes).

Herein is described the production and characterization of 17 MABs that exhibited various degrees of specificities. Some of the MABs can already be applied for differential diagnosis of B virus infections in macaques and humans. Several of these MABs were used for epitope mapping by the Phage Display and Peptide Microarray techniques and lead to the discovery of potentially bioactive B virus peptides. It is hoped that these peptides will enable the design of novel, specific and sensitive serological immunoassays for use in multiple formats for detecting B virus infections in macaque and humans. In addition, it was found that at least one of the MABs has the capacity to neutralize the B virus infectivity in cell cultures. This finding is important because it may lead to the design of better antivirus vaccines and also may facilitate research for the better understanding of virus-host interactions.

While several possible embodiments are disclosed above, embodiments of the disclosure are not so limited. These exemplary embodiments are not intended to be exhaustive or to unnecessarily limit the scope of the disclosure, but instead were chosen and described in order to explain the principles of the disclosure so that others skilled in the art may practice the disclosure. Indeed, various modifications of the disclosure in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

SEQUENCE LISTING

| SEQ | | | |
|---|---|---|---|
| ID | | | |
| NO: | Source | Type | Sequence |
| 1 | Synthetic | Protein | NIVMTQSHEFMSTSVGDRVSISCKASQDVTTAVAWY |
| | | | QQNPGHSPKLLIFSASYRYTGVPERFTGSGSGTDFTFT |
| | | | ISSVQAEDLAVYYCQQHYSAPPTFGGGTKLDIK |
| 2 | Synthetic | Protein | EVQLQQSGPELEKPGASVKISCKASGYSFIGYNMNW |
| | | | VKQSNGKSLEWIGNIDPYYGTTKYNQKFTGKATLTV |
| | | | DKSSSTAYMQLESLTSEDSAVYYCARRGHITPIEVLY |
| | | | FDYWGQGTTLTVSS |
| 3 | Synthetic | Protein | DIVMTQFPSSLSVSPGEKVTMSCKSSQSLLNSGNLVN |
| | | | YLAWYQQKPGQPPKLLIYGASTRESGVPDRFTGSGSG |
| | | | TDFTLTISSVQAEDLAVYYCQNDHIYPLTFGAGTKVE |
| | | | LK |
| 4 | Synthetic | Protein | DVQLQESGPGLVKPSQSLSLTCTVTGYSIASDHAWH |
| | | | WIRQFPGNKLDWMGYISYSGHTYYNPSLKTRISITRD |
| | | | TSKNQFFLQLKSVTSEDTATYYCATMDYWGQGTSVI |
| | | | VSS |
| 5 | Synthetic | Protein | QIVLSQSPAILSASPGERVTMTCRASSSVSYIHWYQQK |
| | | | PGSSPKPWIFATSNLASGVPTRFSGSGSGTSYSLTISRV |
| | | | EAEDAATYYCQQWNSNPLTFGAGTKLELK |
| 6 | Synthetic | Protein | DVQLQESGPGLVKPSQSLSLTCTVTGYSLTSDYAWN |
| | | | WIRQFPGNKLEWMGYIRYSGSTTYNPSLKSRISITRDT |
| | | | SKNQFFLQLNSVTTEDTATYYCASEEISDYSYYHAMD |
| | | | YWGQGTSVTASS |
| 7 | Synthetic | DNA | aacattgtgatgacccagtctcacgaattcatgtccacatcagtaggggacagggtc |
| | | | agcatctcctgcaaggccagtcaggatgtgactactgctgtagcctggtatcaacag |
| | | | aatccaggacattctcctaaattactgatitictcggcatcctaccgatacactggagtc |
| | | | cctgagcgcttcactggcagtggatctgggacggatttcactttcaccatcagcagtg |
| | | | tgcaggctgaagacctggcagtttattactgtcaacaacattatagtgctcctccgacg |
| | | | ttcggtggaggcaccaaactggacatcaaac |
| 8 | Synthetic | DNA | gaggtccagctgcagcagtctggacctgagctggaaaagcctggcgcttcagtgaa |
| | | | gatatcctgcaaggcttctggttactcattcattggctacaacatgaactgggtgaagc |
| | | | agagcaatggaaagagccttgagtggattggaaatattgatccttattatggtactact |
| | | | aagtacaaccagaagttcacgggcaaggccacattgactgtagacaaatcctccag |
| | | | cacagcctacatgcagctcgagagcctgacatctgaggactctgcagtctattactgt |
| | | | gcaagaaggggacacattactccgatagaagtcctatactttgactattggggccaa |
| | | | ggcaccactctcacagtctcctcag |
| 9 | Synthetic | DNA | gacattgtgatgacacagtttccatcctccctgagtgtgtcaccaggagagaaggtca |
| | | | ctatgagctgcaagtccagtcagagtctattaaacagtggaaatcttgtgaactacttg |
| | | | gcctggtaccagcagaaaccaggacaacctcctaaactgttgatctacggggcatc |
| | | | cactagggaatctggggtccctgatcgcttcacaggcagtggatctggcaccgattt |
| | | | cactcttaccatcagcagtgtacaggctgaagacctggcggittattactgtcagaat |
| | | | gatcatatttatcctctcacgttcggtgctgggaccaaggtggagctgaaac |
| 10 | Synthetic | DNA | gatgtgcagcttcaggagtcgggacctggcctggtgaaaccttctcagtctctgtccc |
| | | | tcacgtgcactgtcactggctactcaatcgccagtgatcatgcctggcactggatccg |
| | | | gcagtttccaggaaacaaactggactggatgggctacataagctacagtggtcaca |
| | | | cttactacaacccatctctcaaaactcgaatctctatcactcgagacacatccaagaac |
| | | | cagttcttcttgcaattgaaatctgtgacttctgaggacacagccacatattactgtgcg |
| | | | accatggactactggggtcaaggaacctcagtcatcgtctcctcag |
| 11 | Synthetic | DNA | caaattgttctctcccagtctccagcaatcctgtctgcatctccaggggagagggtca |
| | | | caatgacttgcagggccagctcaagtgtaagttacatacactggtaccagcagaagc |
| | | | caggatcctcccccaaaccctggatttttgccacatccaacctggcttctggagtccct |
| | | | actcgcttcagtggcagtgggictgggacctcttactctctcacaatcagcagagtgg |
| | | | aggctgaagatgctgccacttattactgccagcagtggaatagtaatccgctcacgtt |
| | | | cggtgctgggaccaagctggagctgaaac |
| 12 | Synthetic | DNA | gatgtgcagcttcaggagtcgggacctggcctggtgaaaccttctcagtctctgtccc |
| | | | tcacctgcactgtcactggctactcactcaccagtgattatgcctggaactggatccg |
| | | | gcagtttccaggaaacaaactggagtggatgggctacataaggtacagtggtagca |
| | | | ctacctacaacccatctctcaaaagtcgaatctctatcactcgagacacgtccaagaa |
| | | | ccagttcttcctacagttgaattctgtgactactgaggacacagccacatattactgtgc |
| | | | aagtgaagagattagtgattactcctattaccatgctatggactactggggtcaagga |
| | | | acctcagtcaccgcctcctcag |
| 13 | Synthetic | Protein | DYWSYWWGGGS |
| 14 | Synthetic | Protein | IDKSEYWTYQLRGGGS |
| 15 | Synthetic | Protein | CKHEPYFHCGGGS |
| 16 | Synthetic | Protein | NMANYPLTRPALGGGS |
| 17 | Synthetic | Protein | QPWYQVKGGGS |
| 18 | Synthetic | Protein | QHSFDHYLPLSPGGGS |
| 19 | Synthetic | Protein | SEKEPPVGGGS |
| 20 | Synthetic | Protein | YGEPPVANWEGSGGGS |
| 21 | Synthetic | Protein | CELWPMPFCGGGS |
| 22 | Synthetic | Protein | CTVYPAAWCGGGS |
| 23 | Synthetic | Protein | CYGMMMEWCGGGS |
| 24 | Synthetic | Protein | CTLYPLPWCGGGS |
| 25 | Synthetic | Protein | CYPLGLLGCGGGS |
| 26 | Synthetic | Protein | CGIEIRISGGGS |
| 27 | Synthetic | Protein | CTETDPPLCGGGS |
| 28 | Synthetic | Protein | CVPTAPFQCGGGS |
| 29 | Synthetic | Protein | CAPPAPVQCGGGS |
| 30 | Synthetic | Protein | CYGMMTERCGGGS |
| 31 | Synthetic | Protein | CYGMTPERCGGGS |
| 32 | Synthetic | Protein | CDGMTPEQCGGGS |
| 33 | Synthetic | Protein | TETDPPLGGGS |
| 34 | Synthetic | Protein | WTDETVP |
| 35 | Synthetic | Protein | CDEGVNGYC |
| 36 | Synthetic | Protein | CHDLNGVPC |
| 37 | Synthetic | Protein | ANWSHWEFNRRPGGG |
| 38 | Synthetic | Protein | SWRPGDYGGG |
| 39 | Synthetic | Protein | CWRPGDMACGGG |
| 40 | Synthetic | Protein | GNGCHRGDNRRGGG |
| 41 | Synthetic | Protein | CYEPFWTNC |
| 42 | Synthetic | Protein | ACWAPFWSDC |
| 43 | Synthetic | Protein | ACLHLPWSPC |
| 44 | Synthetic | Protein | DDNDGEAGAAPG |
| 45 | Synthetic | Protein | KYVRNN |
| 46 | Synthetic | Protein | RGWHTTDLK |
| 47 | Synthetic | Protein | FVLATG |
| 48 | Synthetic | Protein | HTAYAADRF |
| 49 | Synthetic | Protein | TLTKWQ |
| 50 | Synthetic | Protein | SFRFSSSALSTTFTT |
| 51 | Synthetic | Protein | FTTNRYEYA |
| 52 | Synthetic | Protein | GTHVKVQV |
| 53 | Synthetic | Protein | ELLREQERRPGD |
| 54 | Synthetic | Protein | ERRPGDAAATPK |
| 55 | Synthetic | Protein | DPPDVERIK |
| 56 | Synthetic | Protein | GDAAATPKPSADPPD |
| 57 | Synthetic | Protein | ASATVG |
| 58 | Synthetic | Protein | NAIASATVGRRV |
| 59 | Synthetic | Protein | QNSMRVPAR |
| 60 | Synthetic | Protein | QNSMRVPARPGT |
| 61 | Synthetic | Protein | EGGPLVEG |
| 62 | Synthetic | Protein | GAGYVYFEDYAYSHQ |
| 63 | Synthetic | Protein | DHEFVPLEV |
| 64 | Synthetic | Protein | ASGGGEEDFD |
| 65 | Synthetic | Protein | RTEHKARKKGTSALL |
| 66 | Synthetic | Protein | KPRYSPLGDTDEEEL |
| 67 | Synthetic | Protein | AVLLSLAVA |
| 68 | Synthetic | Protein | LSLAVALARVPA |
| 69 | Synthetic | Protein | VPAGGG |
| 70 | Synthetic | Protein | RSLTRVNPGRFRGAH |
| 71 | Synthetic | Protein | GAHLAPLEQKTD |
| 72 | Synthetic | Protein | LEQKTDPPDVRR |
| 73 | Synthetic | Protein | VYHVQPFVE |
| 74 | Synthetic | Protein | NPFQTPSVPVAV |
| 75 | Synthetic | Protein | LPRWSFYDN |
| 76 | Synthetic | Protein | TLPLRI |
| 77 | Synthetic | Protein | ACLRGPVFEQGV |
| 78 | Synthetic | Protein | QAAGWH |
| 79 | Synthetic | Protein | GWHGPKAPF |
| 80 | Synthetic | Protein | ELAPEDEDEQAPGDE |
| 81 | Synthetic | Protein | SGHTGAIVGALAGAG |
| 82 | Synthetic | Protein | AAGKHVRLPELLDEG |
| 83 | Synthetic | Protein | ELLDEGPGARRGAP |
| 84 | Synthetic | Protein | ERRPGD |
| 85 | Synthetic | Protein | EDEQAP |
| 86 | Synthetic | Protein | AVLLSLAVALARVPAGGG |

### REFERENCES

1. Katz D., Shi W., Krug P. W., Henkel R., McClure H. and Hilliard J. K. Antibody cross reactivity of alphaherpesviruses as mirrored in naturally infected primates. Arch. Virol. 147: 929-941, 2002.
2. Katz D., Shi W., Krug P, J. K. and Hilliard J. K. Neutralizing Antibody Cross-reactivity of Alphaherpesviruses. Joint Meeting of the 3 Divisions of the International Union of Microbiological Societies 2005, San Francisco, Calif., USA, Jul. 23-28, 2005.
3. Sabin A. B, Wright W. M Acute ascending myelitis following a monkey bite, with isolation of a virus capable of of reproducing the disease. J Exp Med 59: 115-136, 1934.
4. Van Hoosier G. L., Melnick J. L. Neutralizing antibodies in human sera to herpesvirus simiae. Tex Rep Biol Med 19: 376-380, 1961.
5. Eberle R., Hilliard J. The simian herpesviruses. Infect Agents Dis 4:55-70, 1995.
6. Elmore D. and Eberle R. Monkey B virus (Cercopithecine herpesvirus 1). Comp Med 58:11-21, 2008.
7. Weigler B. J. Biology of B virus in macaque and human hosts: a review. Clin Infect Dis 14:555-567, 1992.
8. Calvo C M, Friedlander S. Hilliard J, Swarts R, Nielson J, Dhindsa H, Welch R, Dix R D (2011) Case report:Reactivation of latent B virus (macacine herpesvirus 1) presenting as bilateral uveitis, retinal vasculitis and necrotizing herpetic retinitis. Invest Ophthalmol Vis Sci (Suppl) 51, 2975.
9. Pryor H. W., Chiang H., Raulston G. L., and Melendez L. V. Incidence of neutralizing antibodies to Herpes B virus in the Taiwan monkey (Macaca cyclopis). Primates, 11, 297-301, 1969.
10. Benson M. P., Malane L. S., Banks R., Hicks B. C. and Hilliard J. B Virus (Herpesvirus simiae) and Human Infection. Arch Dermatol. 125, 1247-1248, 1989.
11. Ward J. A., Hilliard J. K. Herpes B virus-specific pathogen-free breeding colonies of macaques: serologic test results and the B-virus status of the macaque. Contemp Top Lab Anim Sci 41:36-41, 2002.
12. Katz D., Shi W., Wildes M. J., Krug P. W., Hilliard J. K. Reassessing the detection of B virus specific antibodies. Comp. Med., 62, 527-526, 2012.
13. Katz D., Shi W., Patrusheva I., Perelygina L., Gowda M. S., Krug P. W., Filfili C. N., Ward J. A., Hilliard J. K. An automated ELISA utilizing recombinant antigens for serologic diagnosis of B virus infections in macaques. Comp. Med., 62, 527-534, 2012.
14. Eberle R., Mou S. W., Zaia J. A. The Immune Response to Herpes Simplex Virus: Comparison of The Specificity and Relative Titers of Serum Antibodies Directed Against Viral Polypeptides Following Primary Herpes Simplex Virus Type Infections. Journal of Medical Virology 16:147-162, 1985.
15. Hogrefe W., Su X, Song J., Ashley R., Kong L. 2002. Detection of Herpes Simplex Virus Type 2-Specific Immunoglobulin G Antibodies in African Sera by Using Recombinant gG2, Western Blotting, and gG2 Inhibition. Journal of clinical microbiology 40:3635-3640, 2002.
16. Katz D., Hilliard J. K, Eberle R., Lipper S. L. ELISA for detection of group-common and virus-specific antibodies in human and simian sera induced by herpes simplex and related simian viruses. J Virol Methods 14:99-109, 1986a.
17. Perelygina L., Patrusheva I., Hombaiah S., Zurkuhlen H., Wildes M. J., Patrushev N., Hilliard J. K. Production of herpes B virus recombinant glycoproteins and evaluation of their diagnostic potential. J Clin Microbiol 43:620-628, 2005.
18. Kohler G. and Milstein C. Continuous cultures of fused cells secreting antibody of preferred specificity. Nature (London) 256: 495-497, 1975.
19. Dean C. and Modjtahedi H. Monoclonal antibodies. In Molecular Biomethods Handbook, eds Rapley R. and Walker J. M; Humana press Inc., Totowa, N.J. Pp. 567-580, 1998.
20. Morris G. E. Epitope mapping. In Molecular Biomethods Handbook, eds Rapley R. and Walker J M; Humana press Inc., Totowa, N.J. Pp. 619-630, 1998.
21. Cropper L. M., Lees D. N., Patt R., Sharp I. R. and Brown D. Monoclonal antibodies for the identification of herpesvirus simiae (B virus). Arch. Virol. 123: 267-77, 1992.
22. Blewett E. L., Black D., Eberle R. Characterization of virus-specific and cross-reactive monoclonal antibodies to Herpesvirus simiae (B virus). J Gen Virol 77: 2787-93, 1996.
23. Smith A. L., Black D. H., Eberle R. Molecular evidence for distinct genotypes of monkey B virus (herpesvirus simiae) which are related to the macaque host species. J Virol 11:9224-9232, 1998.
24. Katz D, Hilliard J K, Eberle R, Lipper S L (1986a) ELISA for detection of group-common and virus-specific antibodies in human and simian sera induced by herpes simplex and related simian viruses. J Virol Methods 14: 99-109.
25. Sofer, G. (2003). Virus Inactivation in the 1990s and into the 21st Century, Part 4, Culture Media, Biotechnology Products, and Vaccines. BioPharm International, January, 50-57.
26. Lin L., Hanson C. V., Alter H. J., Jauviin, V., Bernard K. A., Murthy K. K., Metzel P. and Corash L. Inactivation of viruses in platelet concentrates by a. photochemical treatment with amotosalen and long-wavelength ultraviolet light. Transfusion, 45, 580-590, 2005.
27. Lin L., Dikeman R., Molini B., Lukehart S. A., Lane R., Dupuis K., Metzel P., and Corash L. Photochemical treatment of platelet concentrates with amotosalen and long-wavelength ultraviolet light inactivates a broad spectrum of pathogenic bacteria. Transfusion, 44, 1496-1504, 2004.
28. Jansen G. A. J., Van Vliet H. H. D. M., Vermeij H., Beckers E. A. M., Leebeek F. W. G., Sonneveld P., and van Rhenen D. J. Functional characteristics of photochemically treated platelets. Transfusion, 44, 313-319, 2004.
29. Katz D., Lehrer S. Galan O., Lachmi B. and Cohen S. Adjuvant effects of dimethyl dioctadecyl ammonium bromide, complete Freund's adjuvant and aluminum hydroxide on neutralizing antibody, antibody-isotype and delayed hypersensitivity responses to Semliki Forest virus in mice. FEMS Microbiology Immunology 76: 305-320 1991.
30. Frank R (1992) Spot-synthesis—an easy technique for the positionally addressable, parallel chemical synthesis on a membrane support. Tetrahedron 48: 9217-9232.
31. Dikmans A, Beutling U, Schmeisser E, Thiele S, Frank R (2006) SC2: A novel process for manufacturing multipurpose high-density chemical microarrays. Qsar & Combinatorial Science 25: 1069-1080.
32. Ph.D.™. Phage Display Libraries, Instruction Manual, New England BioLabs® Inc.
33. Norcott J. P., Brown D. W. G. Competitive Radioimmunoassay To Detect Antibodies To Herpes B Virus And Sa8 Virus. Journal Of Clinical Microbiology. (1993) 31:931-935.
34. Hotop S. K., El Wahed A. A., Beutling U., Jentsch D., Motzkus D., Frank R., Hunsmann G., Stahl-Hennig C., Fritz H. J. (2014). Multiple Antibody Targets on Herpes B Glycoproteins B and D Identified by Screening Sera of Infected Rhesus Macaques with Peptide Microarrays. PLOS ONE 9:1-11.

What is claimed is:

1. A monoclonal antibody (MAB) as described in Table 1.

2. A nucleic acid encoding the MAB according to claim 1.

3. A vector comprising the nucleic acid according to claim 2.

4. A host cell comprising the vector according to claim 3.

5. A B virus epitope as described in Table 7.

6. A nucleic acid encoding the B virus epitope according to claim 5.

7. A vector comprising the nucleic acid according to claim 6.

8. A host cell comprising the vector according to claim 7.

9. A monoclonal antibody recognizing the B virus epitope according to claim 5.

11. A recombinant and/or chimeric protein comprising the B virus epitope according to claim 5.

12. A nucleic acid encoding the recombinant and/or chimeric protein according to claim 11.

13. A vector comprising the nucleic acid according to claim 12.

14. A host cell comprising the vector according to claim 13.

15. A pharmaceutical composition comprising any composition of claims 1-14 and combinations thereof.

16. The pharmaceutical composition of claim 15, further comprising a carrier and/or excipients and/or additional therapeutic agent.

17. The pharmaceutical composition of claim 15 or 16, wherein the composition of claims 1-14 is present in a therapeutically effective amount.

18. Use of any composition of claims 1-14 to treat a simplexvirus infection in a subject in need thereof.

19. A method of treatment of a simplexvirus infection in a subject in need thereof, comprising administering any composition of claims 1-14.

20. The method of claim 19, further comprising a diagnostic assay to determine whether the subject has a simplexvirus infection using the antibodies according to claims 1 and 9.

21. The method of claim 20, wherein the diagnostic assay can provide a differential diagnosis of the simplexvirus infection from the group comprising Rh-BV, Cyno-BV, PT-BV, Jap-BV, HSV-1 and/or HSV-2.

22. The pharmaceutical composition of any of claims 15-17, wherein the pharmaceutical composition is a vaccine.

23. The method of any of claims 19-21, wherein the method of treatment comprises administering a vaccine according to claim 22.

24. The method of claim 21, wherein the method comprises treatment of Rh-BV, Jap-BV, PT-BV, and/or Cyno-BV infections with compositions according to any of claims 1-17 comprising: one or more of 7H1.G5, 18D10.F2.A4, 2G12.D12.D4, 6E10.D7, 5D10.C9, 5E10.C10, 7G9.E3, 10F9.F1, 7F7.G7, 5A2, 4E11, 9F1, 3H6, 7G2, 7G8, and/or 1G3;
B virus epitopes recognized by said MABs;
and/or other MABs specific to the B virus epitopes recognized by said MABs.

25. The method of any of claims 19-21, 23 and 24, wherein the method comprises neutralizing the B virus by administration of a composition according to any of claims 1-17 comprising:
5A2 and/or 4E11 MABs;
B virus epitopes recognized by said MABs; and/or
or other MABs specific to the B virus epitopes recognized by said MABs.

26. A method of diagnosing a subject with a simplexvirus infection comprising use of the MABs described in Table 1.

27. A method of diagnosing a subject with a simplexvirus infection comprising use of MABs recognizing the B virus epitopes described in Table 7.

28. The method of claim 26 or 27, wherein the simplexvirus infection is a B virus infection, a HSV-1 infection, and/or a HSV-2 infection.

29. The method of any of claims 26-28, wherein the method further comprises the steps of:
(i) taking a bodily fluid sample (e.g., blood, serum, plasma, urine, saliva, and/or CSF) from a subject;
(ii) determining whether the sample contains a simplexvirus; and
(iii) determining whether the subject is infected with a simplexvirus.

30. The method of any of claims 26-29, wherein the method is used to make a differential diagnosis among Rh-BV, Cyno-BV, PT-BV, Jap-BV, HSV-1, and/or HSV-2.

31. The method of claim 30, wherein the method allows differential diagnosis of Rh-BV and Jap-BV strains by using MABs 12F5.C1 and/or 12G9.G5 as described in Table 1, and/or other MABs recognizing the B virus epitopes of these MABs as described in Table 7.

32. The method of claim 30, wherein the method allows specific diagnosis of one or more of Rh-BV, Cyno-BV, PT-BV, and/or Jap-BV, and not HSV-1 and/or HSV-2, by using one or more of MABs 1G3, 1G3.C1, 9F1, 3H6, and/or 7G2, and/or other MABs recognizing the B virus epitopes of these MABs as described in Table 7.

33. The method of claim 30, wherein the method allows specific diagnosis of one or more of Rh-BV, Cyno-BV, PT-BV, and/or Jap-BV, and not HSV-1 and/or HSV-2, by using one or more of MABs 2G12.D12.D4 and/or 6E10.D7, and/or other MABs recognizing the B virus epitopes of these MABs as described in Table 7.

34. The method of claim 30, wherein the method allows specific diagnosis of one or more of Rh-BV, Cyno-BV, PT-BV, and/or Jap-BV by using one or more of MABs 5E10.C10, 7G8, and/or 7F7.G7, and/or other MABs recognizing the B virus epitopes of these MABs as described in Table 7.

36. The method of claim 30, wherein the method allows differential diagnosis of one or more of Rh-BV, Cyno-BV, PT-BV, Jap-BV, HSV-1 and/or HSV-2 relative to other simian simplexviruses and human simplexviruses by using one or more of MABs 18.D10.F2.A4, 7H1.G5, 5D10.C9, 7G9.E3, 5A2, and/or 4E11, and/or other MABs recognizing the B virus epitopes of these MABs as described in Table 7.

37. The method of any of claims 26-36, wherein the step of determining whether the sample contains simplexviruses involves a hybridization assay.

38. The method of any of claims 26-37, wherein the method further comprises selecting a therapeutic composition and/or method based on the results of step (iii).

39. A B virus-binding fragment that is at least 95%, 96%, 97%, 98%, 99%, or 100% identical to any of SEQ ID NOs: 1-6 or 7-12.

40. An anti-B virus antibody and/or fragment thereof that binds specifically and/or differentially to B viruses and comprise a light chain variable region having an amino acid sequence that is at least 95%, 96%, 97%, 98%, 99%, or 100% identical to any of SEQ ID NOs: 1, 3 and 5, or is a B virus-binding fragment or a homologous variant thereof, of an antibody comprised in said sequences.

41. An anti-B virus antibody and/or fragment thereof that is encoded by a nucleic acid having a nucleic acid sequence that is at least 95%, 96%, 97%, 98%, 99%, or 100% identical to any of SEQ ID NOs: 7, 9 and 11.

42. An anti-B virus antibody and/or fragment thereof that binds specifically and/or differentially to B viruses and comprise a heavy chain variable region having an amino acid sequence that is at least 95%, 96%, 97%, 98%, 99%, or 100% identical to any of SEQ ID NOs: 2, 4, and 6, or is a B virus-binding fragment or a homologous variant thereof, of an antibody comprised in said sequences.

43. An anti-B virus antibody and/or fragment thereof encoded by a nucleic acid having a nucleic acid sequence that is at least 95%, 96%, 97%, 98%, 99%, or 100% identical to any of SEQ ID NOs: 8, 10 and 12.

44. The anti-B virus antibody and/or fragment thereof of any of claims 39-43, wherein the anti-B virus antibody and/or fragment thereof or a B virus-binding fragment or homologous variant thereof is selected from the group consisting of a chimeric antibody, a CDR-grafted or humanized antibody, a single chain antibody, a fusion protein, and a human antibody.

45. An antibody or an antibody fragment that binds to a B virus or a fragment thereof, wherein the antibody or fragment thereof comprises a variable domain comprising a heavy chain (VH) comprising the amino acid sequence of SEQ ID NO: 2, 4, or 6 and a light chain (VL) comprising the amino acid sequence of SEQ ID NO: 1, 3, or 5.

46. An antibody or an antibody fragment that binds to a B virus or a fragment thereof, wherein the VH region may be encoded by a nucleic acid comprising the nucleic acid sequence of SEQ ID NO: 8, 10, or 12 and a light chain (VL) comprising the amino acid sequence of SEQ ID NO: 7, 9, or 11.

47. An antibody or an antibody fragment that binds to a B virus or a fragment thereof, wherein the antibody or fragment thereof comprises:
a VH sequence having at least 95%, 96%, 97%, 98%, 99%, or 100% sequence identity to the amino acid sequence of SEQ ID NO: 2, 4, or 6; and
a VL sequence having at least 9595%, 96%, 97%, 98%, 99%, or 100% sequence identity to the amino acid sequence of SEQ ID NO: 1, 3, or 5.

48. An antibody or an antibody fragment that binds to a B virus or a fragment thereof, wherein the antibody or fragment thereof comprises:
a VH sequence encoded by a nucleic acid having at least 95%, 96%, 97%, 98%, 99%, or 100% sequence identity to the nucleic acid sequence of SEQ ID NO: 8, 10, or 12; and
a VL sequence having at least 95%, 96%, 97%, 98%, 99%, or 100% sequence identity to the amino acid sequence of SEQ ID NO: 7, 9, or 11.

49. An antibody or an antibody fragment thereof that binds to a B virus or a fragment thereof is provided, wherein the antibody comprises a VH comprising the amino acid sequence of SEQ ID NO: 2 (or the VH region may be encoded by a nucleic acid comprising the nucleic acid sequence of SEQ ID NO: 8).

50. The antibody or fragment thereof of claim 49, further comprising a VL comprising the amino acid sequence of SEQ ID NO: 1 (or the VL region may be encoded by a nucleic acid comprising the nucleic acid sequence of SEQ ID NO: 7).

51. The antibody or fragment thereof of claim 49, wherein the antibody or fragment thereof comprises a VH comprising the amino acid sequence of SEQ ID NO: 2 and a VL comprising the amino acid sequence of SEQ ID NO: 1.

52. The antibody or fragment thereof of claim 49, wherein the antibody or fragment thereof comprises a VH encoded by a nucleic acid comprising the nucleic acid sequence of SEQ ID NO: 8 and a VL region encoded by a nucleic acid comprising the nucleic acid sequence of SEQ ID NO: 7.

53. An antibody or an antibody fragment thereof that binds to a B virus or a fragment thereof, wherein the antibody comprises a VH comprising the amino acid sequence of SEQ ID NO: 4 (or the VH region may be encoded by a nucleic acid comprising the nucleic acid sequence of SEQ ID NO: 10).

54. The antibody or fragment thereof of claim 53 further comprising a VL comprising the amino acid sequence of SEQ ID NO: 3 (or the VL region may be encoded by a nucleic acid comprising the nucleic acid sequence of SEQ ID NO: 9).

55. The antibody or fragment thereof of claim 53, wherein the antibody or fragment thereof comprises a VH comprising the amino acid sequence of SEQ ID NO: 4 and a VL comprising the amino acid sequence of SEQ ID NO: 3.

56. The antibody or fragment thereof of claim 53, wherein the antibody or fragment thereof comprises a VH encoded by a nucleic acid comprising the nucleic acid sequence of SEQ ID NO: 10 and a VL region encoded by a nucleic acid comprising the nucleic acid sequence of SEQ ID NO: 9.

57. An antibody or an antibody fragment therof that binds to a B virus or a fragment thereof is provided, wherein the antibody comprises a VH comprising the amino acid sequence of SEQ ID NO: 6 (or the VH region may be encoded by a nucleic acid comprising the nucleic acid sequence of SEQ ID NO: 12).

58. The antibody or fragment thereof of claim 57 further comprising a VL comprising the amino acid sequence of SEQ ID NO: 5 (or the VL region may be encoded by a nucleic acid comprising the nucleic acid sequence of SEQ ID NO: 11).

60. The antibody or fragment thereof of claim 57, wherein the antibody or fragment thereof comprises a VH comprising the amino acid sequence of SEQ ID NO: 6 and a VL comprising the amino acid sequence of SEQ ID NO: 5.

61. The antibody or fragment thereof of claim 57, wherein the antibody or fragment thereof comprises a VH encoded by a nucleic acid comprising the nucleic acid sequence of SEQ ID NO: 12 and a VL region encoded by a nucleic acid comprising the nucleic acid sequence of SEQ ID NO: 11.

62. An antibody or an antibody fragment thereof that binds to a B virus or a fragment thereof, wherein the antibody comprises a VH comprising an amino acid sequence having at least 95%, 96%, 97%, 98%, 99%, or 100% sequence identity to the amino acid sequence of SEQ ID NO: 2 (or the VH region may be encoded by a nucleic acid comprising a nucleic acid sequence having at least 95%, 96%, 97%, 98%, 99%, or 100% sequence identity to the nucleic acid sequence of SEQ ID NO: 8).

63. The antibody or fragment thereof of claim 62, further comprising a VL comprising an amino acid sequence having at least 95%, 96%, 97%, 98%, 99%, or 100% sequence identity to the amino acid sequence of SEQ ID NO: 1 (or the VL region may be encoded by a nucleic acid comprising a nucleic acid sequence having at least 95%, 96%, 97%, 98%, 99%, or 100% sequence identity to the nucleic acid sequence of SEQ ID NO: 7).

64. The antibody or fragment thereof of claim 62, comprising a VH comprising an amino acid sequence having at least 95%, 96%, 97%, 98%, 99%, or 100% sequence identity to the amino acid sequence of SEQ ID NO: 2 and a VL comprising an amino acid sequence having at least 95%, 96%, 97%, 98%, 99%, or 100% sequence identity to the amino acid sequence of SEQ ID NO: 1.

65. The antibody or fragment thereof of claim 62, comprising a VH encoded by a nucleic acid comprising a nucleic acid sequence having at least 95%, 96%, 97%, 98%, 99%, or 100% sequence identity to the nucleic acid sequence of SEQ ID NO: 8 and a VL region encoded by a nucleic acid comprising a nucleic acid sequence having at least 95%, 96%, 97%, 98%, 99%, or 100% sequence identity to the nucleic acid sequence of SEQ ID NO: 7.

66. An antibody or an antibody fragment thereof that binds to a B virus or a fragment thereof, wherein the antibody comprises a VH comprising an amino acid sequence having at least 95%, 96%, 97%, 98%, 99%, or 100% sequence identity to the amino acid sequence of SEQ ID NO: 4 (or the VH region may be encoded by a nucleic acid comprising a nucleic acid sequence having at least 95%, 96%, 97%, 98%, 99%, or 100% sequence identity to the nucleic acid sequence of SEQ ID NO: 10).

67. The antibody or an antibody fragment thereof of claim 66, wherein the antibody or fragment thereof further comprises a VL comprising an amino acid sequence having at least 95%, 96%, 97%, 98%, 99%, or 100% sequence identity to the amino acid sequence of SEQ ID NO: 3 (or the VL region may be encoded by a nucleic acid comprising a nucleic acid sequence having at least 95%, 96%, 97%, 98%, 99%, or 100% sequence identity to the nucleic acid sequence of SEQ ID NO: 9).

68. The antibody or an antibody fragment thereof of claim 66, wherein the antibody or fragment thereof comprises a VH comprising an amino acid sequence having at least 95%, 96%, 97%, 98%, 99%, or 100% sequence identity to the amino acid sequence of SEQ ID NO: 4 and a VL comprising an amino acid sequence having at least 95%, 96%, 97%, 98%, 99%, or 100% sequence identity to the amino acid sequence of SEQ ID NO: 3.

69. The antibody or an antibody fragment thereof of claim 66, wherein the antibody or fragment thereof comprises a VH encoded by a nucleic acid comprising a nucleic acid sequence having at least 95%, 96%, 97%, 98%, 99%, or 100% sequence identity to the nucleic acid sequence of SEQ ID NO: 10 and a VL region encoded by a nucleic acid comprising a nucleic acid sequence having at least 95%, 96%, 97%, 98%, 99%, or 100% sequence identity to the nucleic acid sequence of SEQ ID NO: 9.

70. An antibody or an antibody fragment that binds to a B virus or a fragment thereof, wherein the antibody comprises a VH comprising an amino acid sequence having at least 95%, 96%, 97%, 98%, 99%, or 100% sequence identity to the amino acid sequence of SEQ ID NO: 6 (or the VH region may be encoded by a nucleic acid comprising a nucleic acid sequence having at least 95%, 96%, 97%, 98%, 99%, or 100% sequence identity to the nucleic acid sequence of SEQ ID NO: 12).

71. The antibody or an antibody fragment thereof of claim 70, wherein the antibody or fragment thereof further comprises a VL comprising an amino acid sequence having at least 95%, 96%, 97%, 98%, 99%, or 100% sequence identity to the amino acid sequence of SEQ ID NO: 5 (or the VL region may be encoded by a nucleic acid comprising a nucleic acid sequence having at least 95%, 96%, 97%, 98%, 99%, or 100% sequence identity to the nucleic acid sequence of SEQ ID NO: 11).

72. The antibody or an antibody fragment thereof of claim 70, wherein the antibody or fragment thereof comprises a VH comprising an amino acid sequence having at least 95%, 96%, 97%, 98%, 99%, or 100% sequence identity to the amino acid sequence of SEQ ID NO: 6 and a VL comprising an amino acid sequence having at least 95%, 96%, 97%, 98%, 99%, or 100% sequence identity to the amino acid sequence of SEQ ID NO: 5.

73. The antibody or an antibody fragment thereof of claim 70, wherein the antibody or fragment thereof comprises a VH encoded by a nucleic acid comprising a nucleic acid sequence having at least 95%, 96%, 97%, 98%, 99%, or 100% sequence identity to the nucleic acid sequence of SEQ ID NO: 12 and a VL region encoded by a nucleic acid comprising a nucleic acid sequence having at least 95%, 96%, 97%, 98%, 99%, or 100% sequence identity to the nucleic acid sequence of SEQ ID NO: 11.

74. The anti-B virus antibody and/or fragment thereof of any of claims 39-73, wherein the antibody and/or fragment thereof binds to an epitope within a fragment of a B virus.

75. The anti-B virus antibody and/or fragment thereof of any of claims 39-73, wherein the antibody or fragment thereof binds to an epitope within a fragment of a B virus comprising any of the amino acid sequences provided in Table 7 herein (e.g., SEQ ID NOs: 13-85).

76. The anti-B virus antibody and/or fragment thereof of any of claims 39-73, wherein the anti-B virus antibody and/or fragment thereof or a B virus-binding fragment or homologous variant thereof is selected from the group consisting of a chimeric antibody, a CDR-grafted or humanized antibody, a single chain antibody, a fusion protein, and a human antibody.

77. The anti-B virus antibody and/or fragment thereof of any of claims 39-73, wherein the anti-B virus antibody and/or fragment thereof is a monoclonal antibody.

78. The anti-B virus antibody and/or fragment thereof of any of claims 39-73, wherein the anti-B virus antibody and/or fragment thereof is humanized.

79. The anti-B virus antibody and/or fragment thereof of any of claims 39-73, wherein the anti-B virus antibody and/or fragment thereof is a human antibody.

80. The anti-B virus antibody and/or fragment thereof of any of claims 74-79, wherein at least a portion of the framework sequence of the anti-B virus antibody is a human consensus framework sequence.

81. The anti-B virus antibody and/or fragment thereof of any of claims 74-80, wherein the antibody is an antibody fragment selected from a Fab, Fab′-SH, Fv, scFv, or (Fab′)₂ fragment.

82. A nucleic acid encoding any of the anti-B virus antibodies or fragments thereof of claims 36-81.

83. A vector comprising the nucleic acid of claim 82.

84. A host cell comprising the vector of claim 83.

85. A method of making an anti-B virus antibody or fragment thereof of any of claims 36-73, wherein the method comprises culturing the host cell under conditions suitable for expression of the nucleic acid encoding the antibody, and isolating the antibody.

86. A composition comprising any of the anti-B virus antibodies or fragments thereof of claims 36-85.

87. The composition of claim 86 further comprising a pharmaceutically acceptable carrier and/or an additional therapeutic agent.

88. The composition of claim 87, wherein the additional therapeutic agent is an antiviral agent.

89. A pharmaceutical composition comprising an anti-B virus antibody or fragment thereof

90. The pharmaceutical composition of claim 89 further comprising a pharmaceutically acceptable carrier and/or an additional therapeutic agent.

91. The pharmaceutical composition of claim 90, wherein the additional therapeutic agent is an antiviral agent.

92. The anti-B virus antibody and/or fragment thereof of any of claims 1-38, wherein the anti-B virus antibody and/or fragment thereof is humanized.

93. The anti-B virus antibody and/or fragment thereof of any of claims 1-38, wherein the anti-B virus antibody and/or fragment thereof is a human antibody.

94. The anti-B virus antibody and/or fragment thereof of any of claims 1-38, wherein at least a portion of the framework sequence of the anti-B virus antibody is a human consensus framework sequence.

95. The anti-B virus antibody and/or fragment thereof of any of claims 1-38, wherein the antibody is an antibody fragment selected from a Fab, Fab′-SH, Fv, scFv, or (Fab′)₂ fragment.

96. A method of treatment of a simplexvirus infection in a subject in need thereof, comprising administering any composition of claims 36-95.

97. The method of claim 96, further comprising a diagnostic assay to determine whether the subject has a simplexvirus infection using the antibodies according to any of claim 36-81 or 92-95.

98. The method of claim 97, wherein the diagnostic assay can provide a differential diagnosis of the simplexvirus infection from the group comprising Rh-BV, Cyno-BV, PT-BV, Jap-BV, HSV-1 and/or HSV-2.

99. The pharmaceutical composition of any of claims 89-91, wherein the pharmaceutical composition is a vaccine.

100. The method of any of claims 96-98, wherein the method of treatment comprises administering a vaccine according to claim 99.

101. The method of claim 96, wherein the method comprises treatment of Rh-BV, Jap-BV, PT-BV, and/or Cyno-BV infections with compositions according to any of claims 36-95 comprising: one or more of anti-B virus antibody and/or fragment thereof of any of claim 36-81 or 92-95;
B virus epitopes recognized by said MABs;
and/or other MABs specific to the B virus epitopes recognized by said MABs.

102. The method of any of claims 96-98, 100 and 101, wherein the method comprises neutralizing the B virus by administration of a composition according to any of claims 36-95 comprising:
one or more of anti-B virus antibody and/or fragment thereof of any of claim 36-81 or 92-95;
B virus epitopes recognized by said MABs; and/or
or other MABs specific to the B virus epitopes recognized by said MABs.

103. A method of diagnosing a subject with a simplexvirus infection comprising use of one or more of anti-B virus antibody and/or fragment thereof of any of claim 36-81 or 92-95.

104. The method of claim 103 or 27, wherein the simplexvirus infection is a B virus infection, a HSV-1 infection, and/or a HSV-2 infection.

105. The method of any of claims 103-104, wherein the method further comprises the steps of:
(i) taking a bodily fluid sample (e.g., blood, serum, plasma, urine, saliva, and/or CSF) from a subject;
(ii) determining whether the sample contains a simplexvirus; and
(iii) determining whether the subject is infected with a simplexvirus.

106. The method of any of claims 103-105, wherein the method is used to make a differential diagnosis among Rh-BV, Cyno-BV, PT-BV, Jap-BV, HSV-1, and/or HSV-2.

107. The method of any of claims 103-106, wherein the step of determining whether the sample contains simplexviruses involves a hybridization assay.

108. The method of any of claims 103-107, wherein the method further comprises selecting a therapeutic composition and/or method based on the results of step (iii).


SEQUENCE LISTING (86 sequences)

SEQ ID NO: 1 - protein, 107 aa, Artificial Sequence
  Description of Artificial Sequence Synthetic polypeptide
NIVMTQSHEFMSTSVGDRVSISCKASQDVTTAVAWYQQNPGHSPKLLIFSASYRYTGVPERFTGSGSGTDFTFTISSVQAEDLAVYYCQQHYSAPPTFGGGTKLDIK

SEQ ID NO: 2 - protein, 123 aa, Artificial Sequence
  Description of Artificial Sequence Synthetic polypeptide
EVQLQQSGPELEKPGASVKISCKASGYSFIGYNMNWVKQSNGKSLEWIGNIDPYYGTTKYNQKFTGKATLTVDKSSSTAYMQLESLTSEDSAVYYCARRGHITPIEVLYFDYWGQGTTLTVSS

SEQ ID NO: 3 - protein, 113 aa, Artificial Sequence
  Description of Artificial Sequence Synthetic polypeptide
DIVMTQFPSSLSVSPGEKVTMSCKSSQSLLNSGNLVNYLAWYQQKPGQPPKLLIYGASTRESGVPDRFTGSGSGTDFTLTISSVQAEDLAVYYCQNDHIYPLTFGAGTKVELK

SEQ ID NO: 4 - protein, 112 aa, Artificial Sequence
  Description of Artificial Sequence Synthetic polypeptide
DVQLQESGPGLVKPSQSLSLTCTVTGYSIASDHAWHWIRQFPGNKLDWMGYISYSGHTYYNPSLKTRISITRDTSKNQFFLQLKSVTSEDTATYYCATMDYWGQGTSVIVSS

SEQ ID NO: 5 - protein, 106 aa, Artificial Sequence
  Description of Artificial Sequence Synthetic polypeptide
QIVLSQSPAILSASPGERVTMTCRASSSVSYIHWYQQKPGSSPKPWIFATSNLASGVPTRFSGSGSGTSYSLTISRVEAEDAATYYCQQWNSNPLTFGAGTKLELK

SEQ ID NO: 6 - protein, 123 aa, Artificial Sequence
  Description of Artificial Sequence Synthetic polypeptide
DVQLQESGPGLVKPSQSLSLTCTVTGYSLTSDYAWNWIRQFPGNKLEWMGYIRYSGSTTYNPSLKSRISITRDTSKNQFFLQLNSVTTEDTATYYCASEEISDYSYYHAMDYWGQGTSVTASS

SEQ ID NO: 7 - DNA, 322 bp, Artificial Sequence
  Description of Artificial Sequence Synthetic polynucleotide
aacattgtgatgacccagtctcacgaattcatgtccacatcagtaggggacagggtcagcatctcctgcaaggccagtcaggatgtgactactgctgtagcctggtatcaacagaatccaggacattctcctaaattactgattttctcggcatcctaccgatacactggagtccctgagcgcttcactggcagtggatctgggacggatttcactttcaccatcagcagtgtgcaggctgaagacctggcagtttattactgtcaacaacattatagtgctcctccgacgttcggtggaggcaccaaactggacatcaaac

SEQ ID NO: 8 - DNA, 370 bp, Artificial Sequence
  Description of Artificial Sequence Synthetic polynucleotide
gaggtccagctgcagcagtctggacctgagctggaaaagcctggcgcttcagtgaagatatcctgcaaggcttctggttactcattcattggctacaacatgaactgggtgaagcagagcaatggaaagagccttgagtggattggaaatattgatccttattatggtactactaagtacaaccagaagttcacgggcaaggccacattgactgtagacaaatcctccagcacagcctacatgcagctcgagagcctgacatctgaggactctgcagtctattactgtgcaagaaggggacacattactccgatagaagtcctatactttgactattggggccaaggcaccactctcacagtctcctcag

SEQ ID NO: 9 - DNA, 340 bp, Artificial Sequence
  Description of Artificial Sequence Synthetic polynucleotide
gacattgtgatgacacagtttccatcctccctgagtgtgtcaccaggagagaaggtcactatgagctgcaagtccagtcagagtctattaaacagtggaaatcttgtgaactacttggcctggtaccagcagaaaccaggacaacctcctaaactgttgatctacggggcatccactagggaatctggggtccctgatcgcttcacaggcagtggatctggcaccgatttcactcttaccatcagcagtgtacaggctgaagacctggcggtttattactgtcagaatgatcatatttatcctctcacgttcggtgctgggaccaaggtggagctgaaac

SEQ ID NO: 10 - DNA, 337 bp, Artificial Sequence
  Description of Artificial Sequence Synthetic polynucleotide
gatgtgcagcttcaggagtcgggacctggcctggtgaaaccttctcagtctctgtccctcacgtgcactgtcactggctactcaatcgccagtgatcatgcctggcactggatccggcagtttccaggaaacaaactggactggatgggctacataagctacagtggtcacacttactacaacccatctctcaaaactcgaatctctatcactcgagacacatccaagaaccagttcttcttgcaattgaaatctgtgacttctgaggacacagccacatattactgtgcgaccatggactactggggtcaaggaacctcagtcatcgtctcctcag

SEQ ID NO: 11 - DNA, 319 bp, Artificial Sequence
  Description of Artificial Sequence Synthetic polynucleotide
caaattgttctctcccagtctccagcaatcctgtctgcatctccaggggagagggtcacaatgacttgcagggccagctcaagtgtaagttacatacactggtaccagcagaagccaggatcctcccccaaaccctggatttttgccacatccaacctggcttctggagtccctactcgcttcagtggcagtgggtctgggacctcttactctctcacaatcagcagagtggaggctgaagatgctgccacttattactgccagcagtggaatagtaatccgctcacgttcggtgctgggaccaagctggagctgaaac

SEQ ID NO: 12 - DNA, 370 bp, Artificial Sequence
  Description of Artificial Sequence Synthetic polynucleotide
gatgtgcagcttcaggagtcgggacctggcctggtgaaaccttctcagtctctgtccctcacctgcactgtcactggctactcactcaccagtgattatgcctggaactggatccggcagtttccaggaaacaaactggagtggatgggctacataaggtacagtggtagcactacctacaacccatctctcaaaagtcgaatctctatcactcgagacacgtccaagaaccagttcttcctacagttgaattctgtgactactgaggacacagccacatattactgtgcaagtgaagagattagtgattactcctattaccatgctatggactactggggtcaaggaacctcagtcaccgcctcctcag

SEQ ID NO: 13 - protein, 11 aa, Artificial Sequence
  Description of Artificial Sequence Synthetic peptide
DYWSYWWGGGS

SEQ ID NO: 14 - protein, 16 aa, Artificial Sequence
  Description of Artificial Sequence Synthetic peptide
IDKSEYWTYQLRGGGS

SEQ ID NO: 15 - protein, 13 aa, Artificial Sequence
  Description of Artificial Sequence Synthetic peptide
CKHEPYFHCGGGS

SEQ ID NO: 16 - protein, 16 aa, Artificial Sequence
  Description of Artificial Sequence Synthetic peptide
NMANYPLTRPALGGGS

SEQ ID NO: 17 - protein, 11 aa, Artificial Sequence
  Description of Artificial Sequence Synthetic peptide
QPWYQVKGGGS

SEQ ID NO: 18 - protein, 16 aa, Artificial Sequence
  Description of Artificial Sequence Synthetic peptide
QHSFDHYLPLSPGGGS

SEQ ID NO: 19 - protein, 11 aa, Artificial Sequence
  Description of Artificial Sequence Synthetic peptide
SEKEPPVGGGS

SEQ ID NO: 20 - protein, 16 aa, Artificial Sequence
  Description of Artificial Sequence Synthetic peptide
YGEPPVANWEGSGGGS

SEQ ID NO: 21 - protein, 13 aa, Artificial Sequence
  Description of Artificial Sequence Synthetic peptide
CELWPMPFCGGGS

SEQ ID NO: 22 - protein, 13 aa, Artificial Sequence
  Description of Artificial Sequence Synthetic peptide
CTVYPAAWCGGGS

SEQ ID NO: 23 - protein, 13 aa, Artificial Sequence
  Description of Artificial Sequence Synthetic peptide
CYGMMMEWCGGGS

SEQ ID NO: 24 - protein, 13 aa, Artificial Sequence
  Description of Artificial Sequence Synthetic peptide
CTLYPLPWCGGGS

SEQ ID NO: 25 - protein, 13 aa, Artificial Sequence
  Description of Artificial Sequence Synthetic peptide
CYPLGLLGCGGGS

SEQ ID NO: 26 - protein, 12 aa, Artificial Sequence
  Description of Artificial Sequence Synthetic peptide
CGIEIRISGGGS

SEQ ID NO: 27 - protein, 13 aa, Artificial Sequence
  Description of Artificial Sequence Synthetic peptide
CTETDPPLCGGGS

SEQ ID NO: 28 - protein, 13 aa, Artificial Sequence
  Description of Artificial Sequence Synthetic peptide
CVPTAPFQCGGGS

SEQ ID NO: 29 - protein, 13 aa, Artificial Sequence
  Description of Artificial Sequence Synthetic peptide
CAPPAPVQCGGGS

SEQ ID NO: 30 - protein, 13 aa, Artificial Sequence
  Description of Artificial Sequence Synthetic peptide
CYGMMTERCGGGS

SEQ ID NO: 31 - protein, 13 aa, Artificial Sequence
  Description of Artificial Sequence Synthetic peptide
CYGMTPERCGGGS

SEQ ID NO: 32 - protein, 13 aa, Artificial Sequence
  Description of Artificial Sequence Synthetic peptide
CDGMTPEQCGGGS

SEQ ID NO: 33 - protein, 11 aa, Artificial Sequence
  Description of Artificial Sequence Synthetic peptide
TETDPPLGGGS

SEQ ID NO: 34 - protein, 7 aa, Artificial Sequence
  Description of Artificial Sequence Synthetic peptide
WTDETVP

SEQ ID NO: 35 - protein, 9 aa, Artificial Sequence
  Description of Artificial Sequence Synthetic peptide
CDEGVNGYC

SEQ ID NO: 36 - protein, 9 aa, Artificial Sequence
  Description of Artificial Sequence Synthetic peptide
CHDLNGVPC

SEQ ID NO: 37 - protein, 15 aa, Artificial Sequence
  Description of Artificial Sequence Synthetic peptide
ANWSHWEFNRRPGGG

SEQ ID NO: 38 - protein, 10 aa, Artificial Sequence
  Description of Artificial Sequence Synthetic peptide
SWRPGDYGGG

SEQ ID NO: 39 - protein, 12 aa, Artificial Sequence
  Description of Artificial Sequence Synthetic peptide
CWRPGDMACGGG

SEQ ID NO: 40 - protein, 14 aa, Artificial Sequence
  Description of Artificial Sequence Synthetic peptide
GNGCHRGDNRRGGG

SEQ ID NO: 41 - protein, 9 aa, Artificial Sequence
  Description of Artificial Sequence Synthetic peptide
CYEPFWTNC

SEQ ID NO: 42 - protein, 10 aa, Artificial Sequence
  Description of Artificial Sequence Synthetic peptide
ACWAPFWSDC

SEQ ID NO: 43 - protein, 10 aa, Artificial Sequence
  Description of Artificial Sequence Synthetic peptide
ACLHLPWSPC

SEQ ID NO: 44 - protein, 12 aa, Artificial Sequence
  Description of Artificial Sequence Synthetic peptide
DDNDGEAGAAPG

SEQ ID NO: 45 - protein, 6 aa, Artificial Sequence
  Description of Artificial Sequence Synthetic peptide
KYVRNN

SEQ ID NO: 46 - protein, 9 aa, Artificial Sequence
  Description of Artificial Sequence Synthetic peptide
RGWHTTDLK

SEQ ID NO: 47 - protein, 6 aa, Artificial Sequence
  Description of Artificial Sequence Synthetic peptide
FVLATG

SEQ ID NO: 48 - protein, 9 aa, Artificial Sequence
  Description of Artificial Sequence Synthetic peptide
HTAYAADRF

SEQ ID NO: 49 - protein, 6 aa, Artificial Sequence
  Description of Artificial Sequence Synthetic peptide
TLTKWQ

SEQ ID NO: 50 - protein, 15 aa, Artificial Sequence
  Description of Artificial Sequence Synthetic peptide
SFRFSSSALSTTFTT

SEQ ID NO: 51 - protein, 9 aa, Artificial Sequence
  Description of Artificial Sequence Synthetic peptide
FTTNRYEYA

SEQ ID NO: 52 - protein, 8 aa, Artificial Sequence
  Description of Artificial Sequence Synthetic peptide
GTHVKVQV

SEQ ID NO: 53 - protein, 12 aa, Artificial Sequence
  Description of Artificial Sequence Synthetic peptide
ELLREQERRPGD

SEQ ID NO: 54 - protein, 12 aa, Artificial Sequence
  Description of Artificial Sequence Synthetic peptide
ERRPGDAAATPK

SEQ ID NO: 55 - protein, 9 aa, Artificial Sequence
  Description of Artificial Sequence Synthetic peptide
DPPDVERIK

SEQ ID NO: 56 - protein, 15 aa, Artificial Sequence
  Description of Artificial Sequence Synthetic peptide
GDAAATPKPSADPPD

SEQ ID NO: 57 - protein, 6 aa, Artificial Sequence
  Description of Artificial Sequence Synthetic peptide
ASATVG

SEQ ID NO: 58 - protein, 12 aa, Artificial Sequence
  Description of Artificial Sequence Synthetic peptide
NAIASATVGRRV

SEQ ID NO: 59 - protein, 9 aa, Artificial Sequence
  Description of Artificial Sequence Synthetic peptide
QNSMRVPAR

SEQ ID NO: 60 - protein, 12 aa, Artificial Sequence
  Description of Artificial Sequence Synthetic peptide
QNSMRVPARPGT

SEQ ID NO: 61 - protein, 8 aa, Artificial Sequence
  Description of Artificial Sequence Synthetic peptide
EGGPLVEG

SEQ ID NO: 62 - protein, 15 aa, Artificial Sequence
  Description of Artificial Sequence Synthetic peptide
GAGYVYFEDYAYSHQ

SEQ ID NO: 63 - protein, 9 aa, Artificial Sequence
  Description of Artificial Sequence Synthetic peptide
DHEFVPLEV

SEQ ID NO: 64 - protein, 10 aa, Artificial Sequence
  Description of Artificial Sequence Synthetic peptide
ASGGGEEDFD

SEQ ID NO: 65 - protein, 15 aa, Artificial Sequence
  Description of Artificial Sequence Synthetic peptide
RTEHKARKKGTSALL

SEQ ID NO: 66 - protein, 15 aa, Artificial Sequence
  Description of Artificial Sequence Synthetic peptide
KPRYSPLGDTDEEEL

SEQ ID NO: 67 - protein, 9 aa, Artificial Sequence
  Description of Artificial Sequence Synthetic peptide
AVLLSLAVA

SEQ ID NO: 68 - protein, 12 aa, Artificial Sequence
  Description of Artificial Sequence Synthetic peptide
LSLAVALARVPA

SEQ ID NO: 69 - protein, 6 aa, Artificial Sequence
  Description of Artificial Sequence Synthetic peptide
VPAGGG

SEQ ID NO: 70 - protein, 15 aa, Artificial Sequence
  Description of Artificial Sequence Synthetic peptide
RSLTRVNPGRFRGAH

SEQ ID NO: 71 - protein, 12 aa, Artificial Sequence
  Description of Artificial Sequence Synthetic peptide
GAHLAPLEQKTD

SEQ ID NO: 72 - protein, 12 aa, Artificial Sequence
  Description of Artificial Sequence Synthetic peptide
LEQKTDPPDVRR

SEQ ID NO: 73 - protein, 9 aa, Artificial Sequence
  Description of Artificial Sequence Synthetic peptide
VYHVQPFVE

SEQ ID NO: 74 - protein, 12 aa, Artificial Sequence
  Description of Artificial Sequence Synthetic peptide
NPFQTPSVPVAV

SEQ ID NO: 75 - protein, 9 aa, Artificial Sequence
  Description of Artificial Sequence Synthetic peptide
LPRWSFYDN

SEQ ID NO: 76 - protein, 6 aa, Artificial Sequence
  Description of Artificial Sequence Synthetic peptide
TLPLRI

SEQ ID NO: 77 - protein, 12 aa, Artificial Sequence
  Description of Artificial Sequence Synthetic peptide
ACLRGPVFEQGV

SEQ ID NO: 78 - protein, 6 aa, Artificial Sequence
  Description of Artificial Sequence Synthetic peptide
QAAGWH

SEQ ID NO: 79 - protein, 9 aa, Artificial Sequence
  Description of Artificial Sequence Synthetic peptide
GWHGPKAPF

SEQ ID NO: 80 - protein, 15 aa, Artificial Sequence
  Description of Artificial Sequence Synthetic peptide
ELAPEDEDEQAPGDE

SEQ ID NO: 81 - protein, 15 aa, Artificial Sequence
  Description of Artificial Sequence Synthetic peptide
SGHTGAIVGALAGAG

SEQ ID NO: 82 - protein, 15 aa, Artificial Sequence
  Description of Artificial Sequence Synthetic peptide
AAGKHVRLPELLDEG

SEQ ID NO: 83 - protein, 14 aa, Artificial Sequence
  Description of Artificial Sequence Synthetic peptide
ELLDEGPGARRGAP

SEQ ID NO: 84 - protein, 6 aa, Artificial Sequence
  Description of Artificial Sequence Synthetic peptide
ERRPGD

SEQ ID NO: 85 - protein, 6 aa, Artificial Sequence
  Description of Artificial Sequence Synthetic peptide
EDEQAP

SEQ ID NO: 86 - protein, 18 aa, Artificial Sequence
  Description of Artificial Sequence Synthetic peptide
AVLLSLAVALARVPAGGG